(19) United States
(12) Patent Application Publication
(10) Pub. No.: US 2005/0065133 A1
(43) Pub. Date: Mar. 24, 2005

(54) Pharmaceutical compositions and methods comprising combinations of 2-alkylidene-19-nor-vitamin D derivatives and an EP2 or EP4 selective agonist

(75) Inventors: Andrew G. Lee, Old Lyme, CT (US); David D. Thompson, Gales Ferry, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/944,119

(22) Filed: Sep. 16, 2004

Related U.S. Application Data
(60) Provisional application No. 60/503,798, filed on Sep. 19, 2003.

(51) Int. Cl. A61K 31/59; A61K 31/44

(52) U.S. Cl. 514/167; 514/345

(57) ABSTRACT

The present invention relates to pharmaceutical compositions and methods of treatment comprising administering to a patient in need thereof a combination of a 2-alkylidene-19-nor-vitamin D derivative and an EP₂ or EP₄ selective agonist or a pharmaceutically acceptable salt or prodrug thereof. Particularly, the present invention relates to pharmaceutical compositions and methods comprising administering to a patient in need thereof 2-methylene-19-nor-20(S)-1α,25-dihydroxyvitamin D₃ and an EP₂ or EP₄ selective agonist or a pharmaceutically acceptable salt or prodrug thereof.

### CROSS-REFERENCE TO RELATED APPLICATION

This application claims the benefit from U.S. Provisional Application No. 60/503,798, filed on Sep. 19, 2003.

### FIELD OF THE INVENTION

The present invention relates to pharmaceutical compositions and methods of treatment comprising administering to a patient in need thereof a combination of a 2-alkylidene-19-nor-vitamin D derivative and an EP₂ or EP₄ selective agonist or a pharmaceutically acceptable salt or prodrug thereof. Particularly, the present invention relates to pharmaceutical compositions and methods of treatment comprising administering to a patient in need thereof 2-methylene-19-nor-20(S)-1α,25-dihydroxyvitamin D₃ and an EP₂ or EP₄ selective agonist or a pharmaceutically acceptable salt or prodrug thereof.

### BACKGROUND OF THE INVENTION

Vitamin D is a general term that refers to a group of steroid molecules. The active form of vitamin D, which is called 1,25-dihydroxyvitamin D₃ (1,25-dihydroxycholecalciferol), is biosynthesized in humans by the conversion of 7-dehydrocholesterol to vitamin D₃ (cholecalciferol). This conversion takes place in the skin and requires UV radiation, which is typically from sunlight. Vitamin D₃ is then metabolized in the liver to 25-hydroxyvitamin D₃ (25-hydroxycholecalciferol), which is then further metabolized in the kidneys to the active form of vitamin D, 1,25-dihydroxyitamin D₃. 1,25-dihydroxyvitamin D₃ is then distributed throughout the body where it binds to intracellular vitamin D receptors.

The active form of vitamin D is a hormone that is known to be involved in mineral metabolism and bone growth and facilitates intestinal absorption of calcium.

Vitamin D analogs are disclosed in U.S. Pat. No. 5,843,928, issued Dec. 1, 1998. The compounds disclosed are 2-alkylidene-19-nor-vitamin D derivatives and are characterized by low intestinal calcium transport activity and high bone calcium mobilization activity when compared to 1,25-dihydroxyvitamin D₃.

The present invention provides for methods of treatment using a combination of a 2-alkylidene-19-nor-vitamin D derivative, and particularly the compound 2-methylene-19-nor-20(S)-1α,25-dihydroxyvitamin D₃, (also known as 2MD), and an EP₂ or EP₄ selective agonist or a pharmaceutically acceptable salt or prodrug thereof.

### SUMMARY OF THE INVENTION

The present invention relates to pharmaceutical compositions and methods of treatment comprising administering to a patient in need thereof a combination of a 2-alkylidene-19-nor-vitamin D derivatives and an EP₂ or EP₄ selective agonist or a pharmaceutically acceptable salt or prodrug thereof. Particularly, the present invention relates to pharmaceutical compositions and methods comprising administering to a patient in need thereof 2-methylene-19-nor-20(S)-1α,25-dihydroxyvitamin D₃ and an EP₂ or EP₄ selective agonist or a pharmaceutically acceptable salt or prodrug thereof.

### DETAILED DESCRIPTION OF THE INVENTION

The present invention relates to pharmaceutical compositions and methods of treating metabolic bone disease, senile osteoporosis, postmenopausal osteoporosis, steroid induced osteoporosis, low bone turnover osteoporosis, osteomalacia, renal osteodystrophy, psoriasis, multiple sclerosis, diabetes mellitus, host versus graft rejection, transplant rejection, rheumatoid arthritis, asthma, bone fractures, bone grafts, acne, alopecia, dry skin, insufficient skin firmness, insufficient sebum secretion, wrinkles, hypertension, leukemia, colon cancer, breast cancer, prostate cancer, obesity, osteopenia, male osteoporosis, hypogonadism, andropause, frailty, muscle damage, sarcopenia, osteosarcoma, hypocalcemic tetany, hypoparathyroidism, rickets, vitamin D deficiency, anorexia, low bone mass resulting from aggressive athletic behavior, and for enhancement of peak bone mass in adolescence and prevention of second hip fracture using a combination of a 2-alkylidene-19-nor-vitamin D derivative and an EP₂ or EP₄ selective agonist or a pharmaceutically acceptable salt or prodrug thereof.

In a preferred embodiment, the present invention relates to a method of treating metabolic bone disease, senile osteoporosis, postmenopausal osteoporosis, steroid induced osteoporosis, low bone turnover osteoporosis, osteomalacia, renal osteodystrophy, psoriasis, multiple sclerosis, diabetes mellitus, host versus graft rejection, transplant rejection, rheumatoid arthritis, asthma, bone fractures, bone grafts, acne, alopecia, dry skin, insufficient skin firmness, insufficient sebum secretion, wrinkles, hypertension, leukemia, colon cancer, breast cancer, prostate cancer, obesity, osteopenia, male osteoporosis, hypogonadism, andropause, frailty, muscle damage, sarcopenia, osteosarcoma, hypocalcemic tetany, hypoparathyroidism, rickets, vitamin D deficiency, anorexia, low bone mass resulting from aggressive athletic behavior, and for enhancement of peak bone mass in adolescence and prevention of second hip fracture using 2-methylene-19-nor-20(S)-1α,25-dihydroxyvitamin D₃ and an EP₂ or EP₄ selective agonist or a pharmaceutically acceptable salt or prodrug thereof.

In a preferred embodiment, the methods of treatment using the combination are senile osteoporosis, postmenopausal osteoporosis, bone fractures, bone grafts, breast cancer, prostate cancer, obesity, osteopenia, male osteoporosis, frailty, muscle damage and sarcopenia.

Osteopenia is a thinning of the bones, but less than is seen with osteoporosis and is the stage before true osteoporosis. The World Health Organization has developed diagnostic categories based on bone mass density (BMD) to indicate if a person has normal bones, has osteopenia or has osteoporosis. Normal bone density is within one standard deviation (+1 or −1) of the young adult mean bone density. Osteopenia (low bone mass) is defined as a bone density 1 to 2.5 standard deviations below the young adult mean (−1 to −2.5), and osteoporosis is defined as a bone density which is 2.5 standard deviations or more below the young adult mean (>−2.5).

Hypogonadism is generally defined as inadequate gonadal function, as manifested by deficiencies in gametogenesis and/or the secretion of gonadal hormones, which can result in retardation of puberty and/or reproductive insufficiency. There are three main types of hypogonadism: 1) primary hypogonadism; 2) secondary hypogonadism; and 3) resistance hypogonadism. In primary hypogonadism damage to the Leydig cells impairs androgen production. In secondary hypogonadism disorder of the hypothalamus or pituitary impairs gonadotropin secretion and in resistance hypogonadism, the body response to androgen is inadequate.

Rickets is a childhood disorder involving softening and weakening of the bones, primarily caused by lack of vitamin D, calcium, and/or phosphate.

Anorexia is a disease that has the following characterisitcs: refusal to maintain body weight at or above a minimally normal weight for age and height (e.g., weight loss leading to maintenance of body weight less than 85% of that expected; or failure to make expected weight gain during period of growth, leading to body weight less than 85% of that expected); intense fear of gaining weight or becoming fat, even though underweight; and disturbance in the way in which one's body weight or shape is experienced, undue influence of body weight or shape on self-evaluation, or denial of the seriousness of the current low body weight. The compounds and combinations of the present invention can be used to treat anorexia and can be used to treat bone loss associated with anorexia.

Another condition that can be treated using the compounds and combinations of the present invention is bone loss associated with aggressive athletic behavior, particularly in women. Aggressive participation in exercise, athletics or sports can result in bone loss, which is usually accompanied in women by ammenorhea. Men who also exhibit aggressive athletic behavior also exhibit bone loss.

Andropause (also called male menopause or viropause) is a natural occurrence in men that typically happens between the age of forty and fifty-five. Andropause is a decline in the level of the hormone testosterone. As testosterone levels decline, and men enter andropause, various changes or conditions may be observed including decreased energy and strength, increased body fat, osteoporosis, depression, decreased mental acuity, inability to maintain muscle, cardiovascular disease, atherosclerosis, decreased libido, decreased strength of orgasms, erectile dysfunction, increased irritability, and aching and stiff joints, particularly in the hands and feet. In addition, males undergoing or having undergone andropause can have gynecomastia, serum lipid disorders, including hypercholesterolemia, reduced vascular reactivity, hypogonadism, and benign prostatic hyperplasia.

Frailty is characterized by the progressive and relentless loss of skeletal muscle mass resulting in a high risk of injury from fall, difficulty in recovery from illness, prolongation of hospitalization, and long-term disability requiring assistance in daily living. The reduction of muscle mass, physical strength and physical performance typically leads to diminished quality of life, loss of independence, and mortality. Frailty is normally associated with aging, but may also result when muscle loss and reduced strength occur due to other factors, such as disease-induced cachexia, immobilization, or drug-induced sarcopenia. Another term that has been used to denote frailty is sarcopenia, which is a generic term for the loss of skeletal muscle mass, or quality. Examples of skeletal muscle properties that contribute to its overall quality include contractility, fiber size and type, fatiguability, hormone responsiveness, glucose uptake/metabolism, and capillary density. Loss of muscle quality, even in the absence of loss of muscle mass, can result in loss of physical strength and impaired physical performance.

The term ‘muscle damage’ as used herein is damage to any muscle tissue. Muscle damage can result from physical trauma to the muscle tissue as the result of accidents, athletic injuries, endocrine disorders, disease, wounds or surgical procedures. The methods of the present invention are useful for treating muscle damage by facilitating muscle damage repair.

Osteoporosis in the elderly woman is determined by the amount of peak bone mass gained in adolescence leading to adulthood, the premenopausal maintenance of such peak bone mass, and the rate of postmenopausal bone mass loss. Determinants of peak bone mass include genetic, nutritional, weight loading (exercise), and environmental factors. Enhancement of peak bone mass in adolescence is therefore desirable in order to maximize the skeletal mass in order to prevent the development of osteoporosis later in life. Likewise, enhancement of peak bone mass in adolescence for males is also desirable.

Hip fracture has a significant impact on medical resources and patient morbidity and mortality. Few patients admitted with a hip fracture are considered for prophylactic measures aimed at the reduction of further fracture risk. Currently, 10-13% of patients will later sustain a second hip fracture. Of patients who suffered a second hip fracture, fewer patients maintained their ability to walk independently after the second fracture than did so after the first (53 and 91% respectively, P<0.0005). Pearse E. O. et al., Injury, 2003, 34(7), 518-521. Following second hip fracture, patients' level of mobility determined their future social independence. Older patients and those with a history of multiple falls had a shorter time interval between fractures. Second hip fracture has a significant further impact on patients' mobility and social independence. It is therefore desirable to have new methods for the prevention of second hip fracture.

Osteosarcoma is a relatively common, highly malignant primary bone tumor that has a tendency to metastasize to the lungs. Osteosarcoma is most common in persons 10 to 20, though it can occur at any age. About half of all osteosarcomas are located in the region of the knee but it can be found in any bone. Pain and a mass are the usual symptoms of osteosarcoma. Typical treatment for osteosarcoma is chemotherapy in combination with surgery. Either preoperative or postoperative chemotherapy with agents such as methotrexate, doxorubicin, cisplatin or carboplatin can be used to treat the osteosarcoma.

Hypoparathyroidism is a tendency to hypocalcemia, often associated with chronic tetany resulting from hormone deficiency, characterized by low serum calcium and high serum phosphorus levels. Hypoparathyroidism usually follows accidental removal of or damage to several parathyroid glands during thyroidectomy. Transient hypoparathyroidism is common following subtotal thyroidectomy and occurs permanently in less than three percent of expertly performed thyroidectomies.

Hypocalcemic tetany is a form of tetany resulting from hypocalcemia. Hypocalcemia is characterized by a decrease in total plasma calcium concentration below 8.8 mg/dL (milligrams/deciliter) in the presence of normal plasma protein concentration. Tetany may be overt with spontaneous symptoms or latent. Tetany, when overt, is characterized by sensory symptoms such as paresthesias of the lips, tongue, fingers and feet; carpopedal spasm, which may be prolonged and painful; generalized muscle aching; and spasm of facial musculature. Latent tetany requires provocative tests to elicit and generally occurs at less severely decreased plasma calcium concentrations, such as 7 to 8 mg/dL. Hypocalcemic tetany is also observed in veterinary practice in animals. For example, hypocalcemic tetany in horses is a rare condition associated with acute depletion of serum ionized calcium and sometimes with alterations in serum concentrations of magnesium and phosphate. It occurs after prolonged physical exertion or transport (transport tetany) and in lactating mares (lactation tetany). Signs are variable and relate to neuromuscular hyperirritability.

The present invention is also concerned with pharmaceutical compositions for treating metabolic bone disease, senile osteoporosis, postmenopausal osteoporosis, steroid induced osteoporosis, low bone turnover osteoporosis, osteomalacia, renal osteodystrophy, psoriasis, multiple sclerosis, diabetes mellitus, host versus graft rejection, transplant rejection, rheumatoid arthritis, asthma, bone fractures, bone grafts, acne, alopecia, dry skin, insufficient skin firmness, insufficient sebum secretion, wrinkles, hypertension, leukemia, colon cancer, breast cancer, prostate cancer, obesity, osteopenia, male osteoporosis, hypogonadism, andropause, frailty, muscle damage, sarcopenia, osteosarcoma, hypocalcemic tetany, hypoparathyroidism, rickets, vitamin D deficiency, anorexia, low bone mass resulting from aggressive athletic behavior, and for enhancement of peak bone mass in adolescence and prevention of second hip fracture comprising a 2-alkylidene-19-nor-vitamin D derivative, such as a compound of Formula I, and an EP₂ or EP₄ selective agonist or a pharmaceutically acceptable salt or prodrug thereof, and a carrier, solvent, diluent and the like.

In one embodiment, the combinations of this invention comprise a therapeutically effective amount of a first compound, said first compound being an 2-alkylidene-19-nor-vitamin D derivative, such as a compound of Formula I; and a therapeutically effective amount of a second compound, the second compound being an EP₂ or EP₄ selective agonist or a pharmaceutically acceptable salt or prodrug thereof.

A particularly preferred combination is a combination of 2-methylene-19-nor-20(S)-1α,25-dihydroxyvitamin D₃ and an EP₂ or EP₄ selective agonist or a pharmaceutically acceptable salt or prodrug thereof.

2-Alkylidene-19-nor-vitamin D derivatives that can be used in the present invention are disclosed U.S. Pat. No. 5,843,928, which derivatives are characterized by the general formula I shown below: 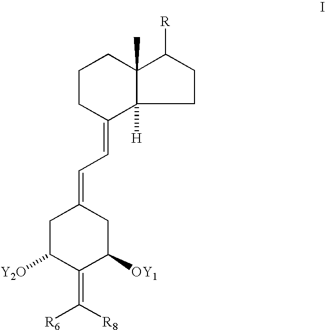
where Y₁ and Y₂, which may be the same or different, are each selected from the group consisting of hydrogen and a hydroxy-protecting group, R₆ and R₈, which may be the same or different, are each selected from the group consisting of hydrogen, alkyl, hydroxyalkyl and fluoroalkyl, or, when taken together represent the group —(CH₂)ₓ— where X is an integer from 2 to 5, and where the group R represents any of the typical side chains known for vitamin D type compounds.

More specifically R can represent a saturated or unsaturated hydrocarbon radical of 1 to 35 carbons, that may be straight-chain, branched or cyclic and that may contain one or more additional substituents, such as hydroxy- or protected-hydroxy groups, fluoro, carbonyl, ester, epoxy, amino or other heteroatomic groups. Preferred side chains of this type are represented by the structure below: 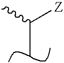
where the stereochemical center (corresponding to C-20 in steroid numbering) may have the R or S configuration (i.e., either the natural configuration about carbon 20 or the 20-epi configuration), and where Z is selected from Y, —OY, —CH₂OY, —C≡CY and —CH═CHY, where the double bond may have the cis or trans geometry, and where Y is selected from hydrogen, methyl, —COR⁵ and a radical of the structure: 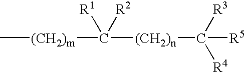
where m and n, independently, represent the integers from 0 to 5, where R¹ is selected from hydrogen, deuterium, hydroxy, protected hydroxy, fluoro, trifluoromethyl, and C₁₋₅-alkyl, which may be straight chain or branched and, optionally, bear a hydroxy or protected-hydroxy substituent, and where each of R², R³ and R⁴, independently, is selected from deuterium, deuteroalkyl, hydrogen, fluoro, trifluoromethyl and C₁₋₅ alkyl, which may be straight-chain or branched, and optionally, bear a hydroxy or protected-hydroxy substituent, and where R¹ and R², taken together, represent an oxo group, or an alkylidene group, ═CR²R³, or the group —(CH₂)ₚ—, where p is an integer from 2 to 5, and where R³ and R⁴, taken together, represent an oxo group, or the group —(CH₂)_{q}—, where q is an integer from 2 to 5, and where R⁵ represent hydrogen, hydroxy, protected hydroxy, or C₁₋₅ alkyl and wherein any of the CH-groups at positions 20, 22 or 23 in the side chain may be replaced by a nitrogen atom, or where any of the groups —CH(CH₃)—, —CH(R³)—, or —CH(R²)— at positions 20, 22 and 23, respectively, may be replaced by an oxygen or sulfur atom.

The wavy line to the methyl substituent at C-20 indicates that carbon 20 may have either the R or S configuration.

Specific important examples of side chains with natural 20R-configuration are the structures represented by formulas (a), (b), (c), (d) and (e) below, i.e., the side chain as it occurs in 25-hydroxyvitamin D₃ (a); vitamin D₃ (b); 25-hydroxyvitamin D₂ (c); vitamin D₂ (d); and the C-24 epimer of 25-hydroxyvitamin D₂ (e);

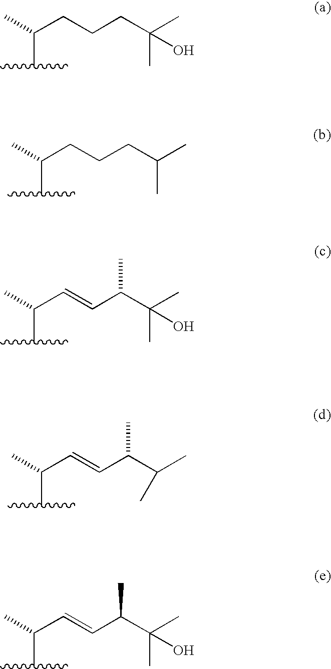

As used herein, the term “hydroxy-protecting group” signifies any group commonly used for the temporary protection of hydroxy functions, such as for example, alkoxycarbonyl, acyl, alkylsilyl or alkylarylsilyl groups (hereinafter referred to simply as “silyl” groups), and alkoxyalkyl groups. Alkoxycarbonyl protecting groups are alkyl-O—CO— groupings such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. The term “acyl” signifies an alkanoyl group of 1 to 6 carbons, in all of its isomeric forms, or a carboxyalkanoyl group of 1 to 6 carbons, such as an oxalyl, malonyl, succinyl, or glutaryl group, or an aromatic acyl group such as benzoyl, or a halo, nitro or alkyl substituted benzoyl group. The word “alkyl” as used in the description or the claims, denotes a straight-chain or branched alkyl radical of 1 to 10 carbons, in all its isomeric forms. Alkoxyalkyl protecting groups are groupings such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals. The term “aryl” specifies a phenyl-, or any alkyl-, nitro- or halo-substituted phenyl group.

A “protected hydroxy” group is a hydroxy group derivatized or protected by any of the above groups commonly used for the temporary or permanent protection of hydroxy functions, e.g., the silyl, alkoxyalkyl, acyl or alkoxycarbonyl groups, as previously defined. The terms “hydroxyalkyl”, “deuteroalkyl” and “fluoroalkyl” refer to any alkyl radical substituted by one or more hydroxy, deuterium or fluoro groups respectively.

It should be noted in this description that the term “24-homo” refers to the addition of one methylene group and the term “24-dihomo” refers to the addition of two methylene groups at the carbon 24 position in the side chain. Likewise, the term “trihomo” refers to the addition of three methylene groups. Also, the term “26,27-dimethyl” refers to the addition of a methyl group at the carbon 26 and 27 positions so that for example R³ and R⁴ are ethyl groups. Likewise, the term “26,27-diethyl” refers to the addition of an ethyl group at the 26 and 27 positions so that R³ and R⁴ are propyl groups.

In the following lists of compounds, the particular alkylidene substituent attached at the carbon 2 position should be added to the nomenclature. For example, if a methylene group is the alkylidene substituent, the term “2-methylene” should precede each of the named compounds. If an ethylene group is the alkylidene substituent, the term “2-ethylene” should precede each of the named compounds, and so on. In addition, if the methyl group attached at the carbon 20 position is in its epi or unnatural configuration, the term “20(S)” or “20-epi” should be included in each of the following named compounds. The named compounds could also be of the vitamin D₂ type if desired.

Specific and preferred examples of the 2-alkylidene-compounds of structure I when the side chain is unsaturated are:
19-nor-24-homo-1,25-dihydroxy-22-dehydrovitamin D₃;
19-nor-24-dihomo-1,25-dihydroxy-22-dehydrovitamin D₃;
19-nor-24-trihomo-1,25-dihydroxy-22-dehydrovitamin D₃;
19-nor-26,27-dimethyl-24-homo-1,25-dihydroxy-22-dehydrovitamin D₃;
19-nor-26,27-dimethyl-24-dihomo-1,25-dihydroxy-22-dehydrovitamin D₃;
19-nor-26,27-dimethyl-24-trihomo-1,25-dihydroxy-22-dehydrovitamin D₃;
19-nor-26,27-diethyl-24-homo-1,25-dihydroxy-22-dehydrovitamin D₃;
19-nor-26,27-diethyl-24-dihomo-1,25-dihydroxy-22-dehydrovitamin D₃;
19-nor-26,27-diethyl,24-trihomo-1,25-dihydroxy-22-dehydrovitamin D₃;
19-nor-26,27-dipropyl-24-homo-1,25-dihydroxy-22-dehydrovitamin D₃;
19-nor-26,27-dipropyl-24-dihomo-1,25-dihydroxy-22-dehydrovitamin D₃; and
19-nor-26,27-dipropyl-24-trihomo-1,25-dihydroxy-22-dehydrovitamin D₃.

Specific and preferred examples of the 2-alkylidene-compounds of structure I when the side chain is saturated are:
19-nor-24-homo-1,25-dihydroxyvitamin D₃;
19-nor-24-dihomo-1,25-dihydroxyvitamin D₃;
19-nor-24-trihomo-1,25-dihydroxyvitamin D₃;
19-nor-26,26-dimethyl-24-homo-1,25-dihydroxyvitamin D₃;
19-nor-26,27-dimethyl-24-dihomo-1,25-dihydroxyvitamin D₃;
19-nor-26,27-dimethyl-24-trihomo-1,25-dihydroxyvitamin D₃;
19-nor-26,27-diethyl-24-homo-1,25-dihydroxyvitamin D₃;
19-nor-26,27-diethyl-24-dihomo-1,25-dihydroxyvitamin D₃;
19-nor-26,27-diethyl-24-trihomo-1,25-dihydroxyvitamin D₃;
19-nor-26,27-dipropyl-24-homo-1,25-dihydroxyvitamin D₃;
19-nor-26,27-dipropyl-24-dihomo-1,25-dihydroxyvitamin D₃; and
19-nor-26,27-dipropyl-24-trihomo-1,25-dihydroxyvitamin D₃.

The present invention also provides such methods wherein the EP₂ selective receptor agonist is a compound of Formula AA 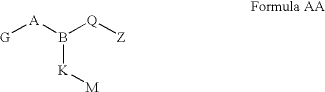
or a prodrug thereof, or a pharmaceutically acceptable salt thereof, wherein
A is SO₂ or CO;
G is Ar, Ar¹—V—Ar², Ar—(C₁-C₆)alkylene, Ar—CONH—(C₁-C₆)alkylene, R¹R²-amino, oxy(C₁-C₆)alkylene, amino substituted with Ar, or amino substituted with Ar(C₁-C₄)alkylene and R¹¹, wherein R¹¹ is H or (C₁-C₈)alkyl, R¹ and R² may be taken separately and are independently selected from H and (C₁-C₈)alkyl, or R¹ and R² are taken together with the nitrogen atom of the amino group to form a five- or six-membered azacycloalkyl, said azacycloalkyl optionally containing an oxygen atom and optionally mono-, di- or tri-substituted independently with up to two oxo, hydroxy, (C₁-C₄)alkyl, fluoro or chloro;
B is N or CH;
Q is
   (C₂-C₆)alkylene-W—(C₁-C₃)alkylene-, said alkylenes each optionally substituted with up to four substituents independently selected from fluoro or (C₁-C₄)alkyl,
   (C₄-C₈)alkylene-, said alkylene optionally substituted with up to four substituents independently selected from fluoro or (C₁-C₄)alkyl,
   X—(C₁-C₅)alkylene-, said alkylene optionally substituted with up to four substituents independently selected from fluoro or (C₁-C₄)alkyl,
   (C₁-C₅)alkylene-X—, said alkylene optionally substituted with up to four substituents independently selected from fluoro or (C₁-C₄)alkyl,
   (C₁-C₃)alkylene-X—(C₁-C₃)alkylene-, said alkylenes each optionally substituted with up to four substituents independently selected from fluoro or (C₁-C₄)alkyl,
   (C₂-C₄)alkylene-W—X—(C₀-C₃)alkylene-, said alkylenes each optionally substituted with up to four substituents each independently selected from fluoro or (C₁-C₄)alkyl,
   (C₀-C₄)alkylene-X—W—(C₁-C₃)alkylene-, said alkylenes each optionally substituted with up to four substituents each independently selected from fluoro or (C₁-C₄)alkyl,
   (C₂-C₅)alkylene-W—X—W—(C₁-C₃)alkylene-, wherein the two occurrences of W are independent of each other, said alkylenes each optionally substituted with up to four substituents each independently selected from fluoro or (C₁-C₄)alkyl,
   (C₁-C₄)alkylene-ethenylene-(C₁-C₄)alkylene-, said alkylenes and said ethenylene each optionally substituted with up to four substituents each independently selected from fluoro or (C₁-C₄)alkyl,
   (C₁-C₄)alkylene-ethenylene-(C₀-C₂)alkylene-X—(C₀-C₅)alkylene-, said alkylenes and said ethenylene each optionally substituted with up to four substituents each independently selected from fluoro or (C₁-C₄)alkyl,
   (C₁-C₄)alkylene-ethenylene-(C₀-C₂)alkylene-X—W—(C₁-C₃)alkylene-, said alkylenes and said ethenylene optionally each substituted with up to four substituents each independently selected from fluoro or (C₁-C₄)alkyl,
   (C₁-C₄)alkylene-ethynylene-(C₁-C₄)alkylene-, said alkylenes and said ethynylene each optionally substituted with up to four substituents each independently selected from fluoro or (C₁-C₄)alkyl, or
   (C₁-C₄)alkylene-ethynylene-X—(C₀-C₃)alkylene-, said alkylenes and said ethynylene each optionally substituted with up to four substituents each independently selected from fluoro or (C₁-C₄)alkyl;
Z is carboxyl, (C₁-C₆)alkoxycarbonyl, tetrazolyl, 1,2,4-oxadiazolyl, 5-oxo-1,2,4-oxadiazolyl, 5-oxo-1,2,4-thiadiazolyl, (C₁-C₄)alkylsulfonylcarbamoyl or phenylsulfonylcarbamoyl;
K is a bond, (C₁-C₉)alkylene, thio(C₁-C₄)alkylene, (C₁-C₄)alkylenethio(C₁-C₄)alkylene, (C₁-C₄)alkyleneoxy(C₁-C₄)alkylene or oxy(C₁-C₄)alkylene, said (C₁-C₉)alkylene optionally mono-unsaturated and wherein, when K is not a bond, K is optionally mono-, di- or tri-substituted independently with chloro, fluoro, hydroxy or methyl;
M is —Ar³, —Ar⁴—V¹—Ar⁵, —Ar⁴—S—Ar⁵, —Ar⁴—SO—Ar⁵, —Ar⁴—SO₂—Ar⁵ or —Ar⁴—O—Ar⁵;
Ar is a partially saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; or Ar is a fully saturated five to seven membered ring having one or two heteroatoms selected independently from oxygen, sulfur and nitrogen;
Ar¹ and Ar² are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur;
said Ar, Ar¹ and Ar² moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³, R⁴ and R⁵ wherein R³, R⁴ and R⁵ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl or mono-N- or di-N,N-(C₁-C₄)alkylaminosulfinyl;
Ar³, Ar⁴ and Ar⁵ are each independently a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, or a tricyclic ring consisting of three fused independently partially saturated, fully saturated or fully unsaturated five or six membered rings, optionally having one to four heteroatoms selected independently from nitrogen, sulfur and oxygen, said partially or fully saturated ring, bicyclic ring or tricyclic ring optionally having one or two oxo groups substituted on carbon or one or two oxo groups substituted on sulfur; said Ar³, Ar⁴ and Ar⁵ moieties are optionally substituted on carbon or nitrogen, on one ring if the moiety is monocyclic, on one or both rings if the moiety is bicyclic, or on one, two or three rings if the moiety is tricyclic, with up to three substituents per moiety independently selected from R³¹, R⁴¹ and R⁵¹ wherein R³¹, R⁴¹ and R⁵¹ are independently hydroxy, nitro, halo, carboxy, (C₁-C₇)alkoxy, (C₁-C₄)alkoxy(C₁-C₄)alkyl, (C₁-C₄)alkoxycarbonyl, (C₁-C₇)alkyl, (C₂-C₇)alkenyl, (C₂-C₇)alkynyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl or mono-N- or di-N,N-(C₁-C₄)alkylaminosulfinyl;
W is oxy, thio, sulfino, sulfonyl, aminosulfonyl-, -mono-N-(C₁-C₄)alkyleneaminosulfonyl-, sulfonylamino, N-(C₁-C₄)alkylenesulfonylamino, carboxamido, N-(C₁-C₄)alkylenecarboxamido, carboxamidooxy, N-(C₁-C₄)alkylenecarboxamidooxy, carbamoyl, -mono-N-(C₁-C₄)alkylenecarbamoyl, carbamoyloxy, or -mono-N-(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, di- or tri-substituted independently with halo, (C₁-C₃)alkyl, trifluoromethyl, trifluoromethyloxy, difluoromethyloxy, hydroxyl, (C₁-C₄)alkoxy, or carbamoyl;
R¹, R², R³, R⁴, R⁵, R¹¹, R³¹, R⁴¹ and R⁵¹, when containing an alkyl, alkylene, alkenylene or alkynylene moiety, are optionally mono-, di- or tri-substituted on carbon independently with halo or hydroxy; and

V and V¹ are each independently a bond, thio(C₁-C₄)alkylene, (C₁-C₄)alkylenethio, (C₁-C₄)alkyleneoxy, oxy(C₁-C₄)alkylene or (C₁-C₃)alkylene optionally mono- or di-substituted independently with hydroxy or fluoro;
with the provisos that:
a. when K is (C₂-C₄)alkylene and M is Ar³ and Ar³ is cyclopent-1-yl, cyclohex-1-yl, cyclohept-1-yl or cyclooct-1-yl then said (C₅-C₈)cycloalkyl substituents are not substituted at the one position with hydroxy; and
b. when K is a bond; G is phenyl, phenylmethyl, substituted phenyl or substituted phenylmethyl; Q is (C₃-C₈)alkylene; and M is Ar³ or Ar⁴—Ar⁵, then A is sulfonyl.

Examples of EP₂ selective receptor agonists of Formula AA are set forth in U.S. Pat. No. 6,498,172. A preferred EP₂ selective receptor agonist that can be used in the present methods is a compound of Formula M as defined above.

A preferred group of compounds designated the A Group, comprises those compounds having the Formula AA as shown above, prodrugs thereof and pharmaceutically acceptable salts of said compounds and said prodrugs, wherein B is N; Z is carboxyl, (C₁-C₆)alkoxycarbonyl or tetrazolyl; Ar is phenyl, furyl, thienyl, pyrrolyl, oxazolyl, thiazolyl, imidazolyl, pyrazolyl, isoxazolyl, isothiazolyl, pyridyl, pyridazinyl, pyrimidinyl, pyrazinyl, 2H-pyrrolyl, 3H-pyrrolyl, pyrrolyl, 2-pyrrolinyl, 3-pyrrolinyl, pyrrolidinyl, 1,3-dioxolanyl, 2H-imidazolyl, 2-imidazolinyl, imidazolidinyl, 2-pyrazolinyl, pyrazolidinyl, 1,2,3-oxadiazolyl, 1,2,4-oxadiazolyl, 1,2,5-oxadiazolyl, 1,3,4-oxadiazolyl, 1,2,3-triazolyl, 1,2,4-triazolyl, 2H-pyranyl, 4H-pyranyl, pyridyl, piperidinyl, 1,4-dioxanyl, morpholinyl, 1,4-dithianyl, thiomorpholinyl, piperazinyl, 1,3,5-triazinyl, 1,2,4-triazinyl, azepinyl, oxepinyl, thiepinyl, cyclopentenyl, cyclohexenyl, benzo(b)thienyl, benzoxazolyl, benzimidazolyl, benzthiazolyl, quinolinyl, isoquinolinyl, phthalazinyl, quinazolinyl, quinoxalinyl, naphthyl, tetralinyl, decalinyl, 2H-1-benzopyranyl and 1,4-benzodioxan; Ar¹, Ar², Ar³, Ar⁴ and Ar⁵ are each independently cyclopentyl, cyclohexyl, cycloheptyl, cyclooctyl, phenyl, furyl, thienyl, pyrrolyl, oxazolyl, thiazolyl, imidazolyl, pyrazolyl, isoxazolyl, isothiazolyl, pyridyl, pyridazinyl, pyrimidinyl, pyrazinyl, 2H-pyrrolyl, 3H-pyrrolyl, pyrrolyl, 2-pyrrolinyl, 3-pyrrolinyl, pyrrolidinyl, 1,3-dioxolanyl, 2H-imidazolyl, 2-imidazolinyl, imidazolidinyl, 2-pyrazolinyl, pyrazolidinyl, 1,2,3-oxadiazolyl, 1,2,4-oxadiazolyl, 1,2,5-oxadiazolyl, 1,3,4-oxadiazolyl, 1,2,3-triazolyl, 1,2,4-triazolyl, 2H-pyranyl, 4H-pyranyl, pyridyl, piperidinyl, 1,4-dioxanyl, morpholinyl, 1,4-dithianyl, thiomorpholinylpiperazinyl, 1,3,5-triazinyl, 1,2,4-triazinyl, azepinyl, oxepinyl, thiepinyl, 1,2,4-diazepinyl, cyclopentenyl, cyclohexenyl, cycloheptenyl, cyclooctenyl, cyclooctadienyl, indolizinyl, indolyl, isoindolyl, 3H-indolyl, 1H-isoindolyl, indolinyl, cyclopenta(b)pyridinyl, pyrano(3,4-b)pyrrolyl, benzofuryl, isobenzofuryl, benzo(b)thienyl, benzo(c)thienyl, 1H-indazolyl, indoxazinyl, benzoxazolyl, anthranilyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2H-1-benzopyranyl, 1,4-benzodioxan, pyrido(3,4-b)-pyridinyl, pyrido(3,2-b)-pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl; and X is tetrahydrofuranyl, phenyl, thiazolyl, thienyl, pyridyl, pyrrazolyl, furanyl or pyrimidyl, wherein X is optionally mono-, di- or tri-substituted independently with chloro, fluoro, methoxy, difluoromethoxy, trifluoromethoxy, trifluoromethyl or methyl; and wherein each of said Ar, Ar¹ and Ar² groups are optionally substituted on carbon or nitrogen with up to three substituents independently selected from R³, R⁴ and R⁵; each of said Ar, Ar¹ and Ar² groups are optionally substituted independently on carbon or sulfur with one or two oxo groups; each of said Ar³, Ar⁴ and Ar⁵ groups are optionally substituted on carbon or nitrogen independently with up to three R³¹, R⁴¹ and R⁵¹ groups and each of said Ar³, Ar⁴ and Ar⁵ groups are optionally substituted independently on carbon or sulfur with one or two oxo groups.

A group of compounds within the A Group, designated the B Group, comprises those compounds, prodrugs thereof and pharmaceutically acceptable salts of said compounds and said prodrugs, wherein A is CO; G is oxy(C₁-C₆)alkylene; Q is
(C₂-C₆)alkylene-O—(C₁-C₃)alkylene-,
(C₄-C₈)alkylene-, said —(C₄-C₈)alkylene- optionally substituted with up to four substituents independently selected from fluoro or (C₁-C₄)alkyl,
X—(C₂-C₅)alkylene-,
(C₁-C₅)alkylene-X—,
(C₁-C₃)alkylene-X—(C₁-C₃)alkylene-,
(C₂-C₄)alkylene-O—X—(C₀-C₃)alkylene-, or
(C₀-C₄)alkylene-X—O—(C₁-C₃)alkylene-; and X is phenyl, thienyl, furanyl or thiazolyl, wherein X is optionally mono-, di- or tri-substituted with chloro, fluoro, methoxy, difluoromethoxy, trifluoromethoxy, trifluoromethyl or methyl.

Another group of compounds which is preferred within the A Group, designated the C Group, comprises those compounds, prodrugs thereof and pharmaceutically acceptable salts of said compounds and said prodrugs, wherein A is CO; G is Ar; Q is
(C₂-C₆)alkylene-O—(C₁-C₃)alkylene-,
(C₄-C₈)alkylene-, said —(C₄-C₈)alkylene- optionally substituted with up to four substituents independently selected from fluoro or (C₁-C₄)alkyl,
X—(C₂-C₅)alkylene-,
(C₁-C₅)alkylene-X—,
(C₁-C₃)alkylene-X—(C₁-C₃)alkylene-,
(C₂-C₄)alkylene-O—X—(C₀-C₃)alkylene-, or
(C₀-C₄)alkylene-X—O—(C₁-C₃)alkylene-; and X is phenyl, thienyl, furanyl or thiazolyl, wherein X is optionally mono-, di- or tri-substituted with chloro, fluoro, methoxy, difluoromethoxy, trifluoromethoxy, trifluoromethyl or methyl.

Another group of compounds which is preferred within the A Group, designated the D Group, comprises those compounds, prodrugs thereof and pharmaceutically acceptable salts of said compounds and said prodrugs, wherein A is CO; G is R¹R²-amino or amino substituted with Ar, or amino substituted with Ar(C₁-C₄)alkylene and R¹¹, wherein R¹¹ is H; Q is
(C₂-C₆)alkylene-O—(C₁-C₃)alkylene-,
(C₄-C₈)alkylene-, said —(C₄-C₈)alkylene- optionally substituted with up to four substituents independently selected from fluoro or (C₁-C₄)alkyl,
X—(C₂-C₅)alkylene-,
(C₁-C₅)alkylene-X—,
(C₁-C₃)alkylene-X—(C₁-C₃)alkylene-,
(C₂-C₄)alkylene-O—X—(C₀-C₃)alkylene-, or
(C₀-C₄)alkylene-X—O—(C₁-C₃)alkylene-; and X is phenyl, thienyl, furanyl or thiazolyl, wherein X is optionally mono-, di- or tri-substituted with chloro, fluoro, methoxy, difluoromethoxy, trifluoromethoxy, trifluoromethyl or methyl; and
wherein R¹ and R² may be taken separately and are independently selected from H and (C₁-C₈)alkyl, or R¹ and R² are taken together to form a five- or six-membered azacycloalkyl, said azacycloalkyl optionally containing an oxygen atom.

Another group of compounds which is preferred within the G Group, designated the E Group, comprises those compounds, prodrugs thereof and pharmaceutically acceptable salts of said compounds and said prodrugs, wherein A is SO₂; G is R¹R²-amino, or amino substituted with Ar and R¹¹; Q is
(C₂-C₆)alkylene-O—(C₁-C₃)alkylene-,
(C₄-C₈)alkylene-, said —(C₄-C₈)alkylene- optionally substituted with up to four substituents independently selected from fluoro or (C₁-C₄)alkyl,
X—(C₂-C₅)alkylene-,
(C₁-C₅)alkylene-X—,
(C₁-C₃)alkylene-X—(C₁-C₃)alkylene-,
(C₂-C₄)alkylene-O—X—(C₀-C₃)alkylene-, or
(C₀-C₄)alkylene-X—O—(C₁-C₃)alkylene-; and X is phenyl, thienyl, furanyl or thiazolyl, wherein X is optionally mono-, di- or tri-substituted with chloro, fluoro, methoxy, difluoromethoxy, trifluoromethoxy, trifluoromethyl or methyl; and
wherein R¹ and R² may be taken separately and are independently selected from H and (C₁-C₈)alkyl, or R¹ and R² are taken together to form a five- or six-membered azacycloalkyl, said azacycloalkyl optionally containing an oxygen atom.

Another group of compounds which is preferred within the A Group, designated the F Group, comprises those compounds, prodrugs thereof and pharmaceutically acceptable salts of said compounds and said prodrugs, wherein A is SO₂; G is Ar, Ar(C₁-C₂)alkylene or Ar¹—V—Ar²; Q is
(C₂-C₆)alkylene-O—(C₁-C₃)alkylene-,
(C₄-C₈)alkylene-, said —(C₄-C₈)alkylene- optionally substituted with up to four substituents independently selected from fluoro or (C₁-C₄)alkyl,
X—(C₂-C₅)alkylene-,
(C₁-C₅)alkylene-X—,
(C₁-C₃)alkylene-X—(C₁-C₃)alkylene-,
(C₂-C₄)alkylene-O—X—(C₀-C₃)alkylene-, or
(C₀-C₄)alkylene-X—O—(C₁-C₃)alkylene-; and X is phenyl, pyrimidyl, pyridyl, thienyl, tetrahydrofuranyl, furanyl or thiazolyl, wherein X is optionally mono-, di- or tri-substituted with chloro, fluoro, methoxy, difluoromethoxy, trifluoromethoxy, trifluoromethyl or methyl.

A particularly preferred group of compounds within the F Group, designated the FA Group, comprises those compounds, prodrugs thereof and pharmaceutically acceptable salts of said compounds and said prodrugs, wherein G is Ar or Ar—(C₁-C₂)-alkylene; Ar is phenyl, furyl, thienyl, pyrrolyl, oxazolyl, thiazolyl, imidazolyl, pyrazolyl, isoxazolyl, pyridyl, pyridazinyl, pyrimidinyl, pyrazinyl, isothiazolyl, 1,2,3-triazolyl, 1,2,4-triazolyl or 1,3,4-thiadiazolyl wherein each of said Ar groups is optionally substituted on carbon or nitrogen with R¹, R² or R³; Ar⁴ is cyclopentyl, cyclohexyl, cycloheptyl, cyclooctyl, phenyl, furyl, thienyl, pyrrolyl, oxazolyl, thiazolyl, imidazolyl, pyrazolyl, isoxazolyl, isothiazolyl, pyridyl, pyridazinyl, pyrimidinyl, pyrazinyl, pyrrolidinyl, 1,2,3-triazolyl, 1,2,4-triazolyl, pyranyl, thiomorpholinyl, piperazinyl, 1,3,5-triazinyl, 1,2,4-triazinyl, 1,2,3-triazinyl, azepinyl, oxepinyl or thiepinyl wherein each of said Ar⁴ groups is optionally mono- di- or tri-substituted on carbon or nitrogen with R³¹, R⁴¹ or R⁵¹; Ar⁵ is cyclopentyl, cyclohexyl, cycloheptyl, cyclooctyl, phenyl, furyl, thienyl, pyrrolyl, oxazolyl, thiazolyl, imidazolyl, pyrazolyl, isoxazolyl, isothiazolyl, pyridyl, pyridazinyl, pyrimidinyl, pyrazinyl, pyrrolidinyl, 1,2,3-triazolyl, 1,2,4-triazolyl, pyranyl, 1,4-dioxanyl, thiomorpholinyl, piperazinyl, 1,3,5-triazinyl, 1,2,4-triazinyl, 1,2,3-triazinyl, azepinyl, oxepinyl or thiepinyl wherein each of said Ar⁵ groups is optionally mono- di- or tri-substituted on carbon or nitrogen with R³, R⁴¹ or R⁵¹; Q is —(C₅-C₇)-alkylene-, —(C₁-C₂)-alkylene-X—(C₁-C₂)-alkylene-, —(C₁-C₂)—X—O—(C₁-C₂)-alkylene-, —(C₂-C₄)-alkylene-thienyl-, —(C₂-C₄)-alkylene-furanyl- or —(C₂-C₄)-alkylene-thiazolyl-; X is phenyl, pyridyl, pyrimidyl or thienyl; and said X groups are optionally mono-, di- or tri-substituted with chloro, fluoro, methoxy, difluoromethoxy, trifluoromethoxy, trifluoromethyl or methyl; said —(C₂-C₄)-alkylene-furanyl- and —(C₂-C₄)-alkylene-thienyl- having a 2,5-substitution pattern, e.g.,

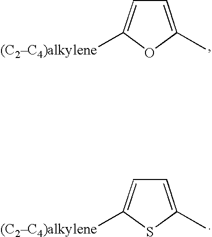

A preferred group of compounds within the FA Group, designated the FB Group, comprises those compounds, prodrugs thereof and pharmaceutically acceptable salts of said compounds and said prodrugs, wherein K is methylene, M is Ar⁴—Ar⁵, Ar⁴—O—Ar⁵ or Ar⁴—S—Ar⁵ and Ar is phenyl, pyridyl, pyrazolyl, imidazolyl, pyrimidyl, thienyl or thiazolyl, wherein Ar is optionally mono-, di- or tri-substituted on carbon or nitrogen with R³, R⁴ or R⁵.

A preferred group of compounds within the FB Group, designated the FC Group, comprises those compounds, prodrugs thereof and pharmaceutically acceptable salts of said compounds and said prodrugs, wherein M is Ar⁴—Ar⁵; Ar is phenyl, pyridyl or imidazolyl; Ar⁴ is phenyl, furanyl or pyridyl; and Ar⁵ is cyclopentyl, cyclohexyl, cycloheptyl, phenyl, pyridyl, imidazolyl, pyrimidyl, thienyl, pyridazinyl, pyrazinyl, imidazolyl, pyrazolyl or thiazolyl, wherein Ar, Ar⁴ and Ar⁵ are optionally mono, -di- or tri-substituted on carbon or nitrogen independently with chloro, fluoro, methyl, methoxy, difluoromethoxy, trifluoromethyl or trifluoromethoxy.

An especially preferred group of compounds within the FC Group, designated the FD Group, comprises those compounds, prodrugs thereof and pharmaceutically acceptable salts of said compounds and said prodrugs, wherein Q is —(C₅-C₇)alkylene-.

Another especially preferred group of compounds within the FC Group, designated the FE Group, comprises those compounds, prodrugs thereof and pharmaceutically acceptable salts of said compounds and said prodrugs, wherein Q is CH₂—X—CH₂— and X is metaphenylene optionally mono- or di-substituted with chloro, fluoro, methoxy, difluoromethoxy, trifluoromethoxy, trifluoromethyl or methyl.

A preferred group of compounds within the FE Group are those compounds, and pharmaceutically acceptable salts and prodrugs thereof, selected from (3-(((pyridine-3-sulfonyl)-(4-pyrimidin-5-yl-benzyl)-amino)-methyl)-phenyl)-acetic acid; (3-(((5-phenyl-furan-2-ylmethyl)-(pyridine-3-sulfonyl)-amino)-methyl)-phenyl)-acetic acid; (3-(((pyridine-3-sulfonyl)-(4-pyrimidin-2-yl-benzyl)-amino)-methyl)-phenyl)-acetic acid; (3-(((pyridine-3-sulfonyl)-(4-thiazol-2-yl-benzyl)-amino)-methyl)-phenyl)acetic acid; and (3-(((4-pyrazin-2-yl-benzyl)-(pyridine-3-sulfonyl)-amino)-methyl)-phenyl)-acetic acid.

An especially preferred compound within the FE Group is the compound wherein Ar is pyrid-3-yl; Z is carboxy; M is Ar⁴—Ar⁵ wherein Ar⁴ is a furanyl ring and Ar⁵ is phenyl wherein said phenyl moiety is substituted at the 5-position of said furanyl ring; and Q is —CH₂—X—CH₂— wherein X is metaphenylene.

Another especially preferred compound within the FE Group is the compound wherein Ar is pyrid-3-yl; Z is carboxy; M is Ar⁴—Ar⁵ wherein Ar⁴ is phenyl and Ar⁵ is pyrimid-2-yl and said pyrimid-2-yl moiety is substituted at the 4-position of said phenyl ring; and Q is —CH₂—X—CH₂— wherein X is metaphenylene.

Yet another especially preferred compound within th FE Group is the compound wherein Ar is pyrid-3-yl; Z is carboxy; M is Ar⁴—Ar⁵ wherein Ar⁴ is phenyl and Ar⁵ is thiazol-2-yl and said thiazol-2-yl moiety is substituted at the 4-position of said phenyl ring; and Q is —CH₂—X—CH₂— wherein X is metaphenylene.

Yet another especially preferred compound within the FE Group is the compound wherein Ar is pyrid-3-yl; Z is carboxy; M is Ar⁴—Ar⁵ wherein Ar⁴ is phenyl and Ar⁵ is pyrimid-5-yl and said pyrimid-5-yl moiety is substituted at the 4-position of said phenyl ring; and Q is —CH₂—X—CH₂— wherein X is metaphenylene.

Yet another especially preferred compound within the FE Group is the compound wherein Ar is pyrid-3-yl; Z is carboxy; M is Ar⁴—Ar⁵ wherein Ar⁴ is phenyl and Ar⁵ is pyrazin-2-yl and said pyrazin-2-yl is substituted at the 4-position of said phenyl ring; and Q is —CH₂—X—CH₂— wherein X is metaphenylene.

A preferred group of compounds within the FC Group, designated the G Group, comprises those compounds, prodrugs thereof and pharmaceutically acceptable salts of said compounds and said prodrugs, wherein Q is —(C₂-C₄)-alkylene-thienyl-, —(C₂-C₄)-alkylene-furanyl- or —(C₂-C₄)-alkylene-thiazolyl-.

An especially preferred compound within the G Group is 5-(3-((pyridine-3-sulfonyl)-(4-thiazol-2-yl-benzyl)-amino)-propyl)-thiophene-2-carboxylic acid.

An especially preferred compound within the G Group is the compound, prodrugs thereof and pharmaceutically acceptable salts of said compounds and said prodrugs, wherein Q is n-propylenyl; X is thienyl; Z is carboxy; Ar is 3-pyridyl; Ar⁴ is phenyl; and Ar⁵ is 2-thiazolyl; said 2-thiazolyl being substituted at the 4-position of said phenyl.

Another especially preferred group of compounds within the FC Group, designated the H Group, comprises those compounds, prodrugs thereof and pharmaceutically acceptable salts of said compounds and said prodrugs, wherein Q is —CH₂—X—O—CH₂—; Ar⁴ is phenyl or pyridyl; said phenyl and pyridyl are optionally substituted with chloro, fluoro, methoxy, difluoromethoxy, trifluoromethoxy, trifluoromethyl and methyl; and X is metaphenylene.

A preferred group of compounds within the H Group are (3-(((4-cyclohexyl-benzyl)-(pyridine-3-sulfonyl)-amino)-methyl)-phenoxy)-acetic acid; (3-(((pyridine-3-sulfonyl)-(4-pyridin-2-yl-benzyl)-amino)-methyl)-phenoxy)-acetic acid; (3-(((pyridine-3-sulfonyl)-(4-pyridin-3-yl-benzyl)-amino)-methyl)-phenoxy)-acetic acid; (3-(((pyridine-3-sulfonyl)-(4-pyridin-4-yl-benzyl)-amino)-methyl)-phenoxy)-acetic acid; and (3-(((pyridine-3-sulfonyl)-(4-thiazol-2-yl-benzyl)-amino)-methyl)-phenoxy)-acetic acid.

An especially preferred compound within the H Group is the compound, prodrugs thereof and pharmaceutically acceptable salts of said compounds and said prodrugs, wherein Ar is pyrid-3-yl; Z is carboxy; Ar⁴ is phenyl; Ar⁵ is cyclohexyl; and said cyclohexyl moiety is substituted at the 4-position of said phenyl ring.

Another especially preferred compound within the H Group is the compound wherein Ar is pyrid-3-yl; Z is carboxy; Ar⁴ is phenyl; Ar⁵ is thiazol-2-yl; and said thiazol-2-yl moiety is substituted at the 4-position of said phenyl ring.

Yet another especially preferred compound within the H Group is the compound wherein Ar is pyrid-3-yl; Z is carboxy; Ar⁴ is phenyl; Ar⁵ is 2-pyridyl; and said 2-pyridyl moiety is substituted at the 4-position of said phenyl ring.

Yet another especially preferred compound within the H Group is the compound wherein Ar is pyrid-3-yl; Z is carboxy; Ar⁴ is phenyl; Ar⁵ is 3-pyridyl; and said 3-pyridyl moiety is substituted at the 4-position of said phenyl ring.

Yet another especially preferred compound within the H Group is the compound wherein Ar is pyrid-3-yl; Z is carboxy; Ar⁴ is phenyl; Ar⁵ is 4-pyridyl; and said 4-pyridyl moiety is substituted at the 4-position of said phenyl ring.

A preferred group of compounds within the FA Group, designated the I Group, comprises those compounds, prodrugs thereof and pharmaceutically acceptable salts of said compounds and said prodrugs, wherein K is methylene, G is Ar; Ar is phenyl, pyridazinyl, pyrazolyl, pyrazinyl, pyridyl, imidazolyl, pyrimidyl, thienyl or thiazolyl, Ar is optionally mono-, di- or tri-substituted with R³, R⁴ or R⁵, and M is Ar³, wherein said Ar³ is cyclopentyl, cyclohexyl, phenyl, thienyl, pyridazinyl, pyrimidinyl, pyrazinyl, indolyl, benzofuryl, benzo(b)thienyl, benzoxazolyl, benzthiazolyl, quinolinyl, isoquinolinyl, naphthyl, tetralinyl, 2H-1-benzopyranyl or 1,4-benzodioxan and is optionally mono-, di- or tri-substituted with R³, chloro, fluoro, methyl, methoxy, difluoromethoxy, trifluoromethyl or trifluoromethoxy.

An especially preferred group of compounds within the I Group are (3-(((2,3-dihydro-benzo[1,4]dioxin-6-ylmethyl)-(pyridine-3-sulfonyl)-amino)-methyl)-phenyl)-acetic acid; and (3-((benzofuran-2-ylmethyl-(pyridine-3-sulfonyl)-amino)-methyl)-phenyl)-acetic acid.

An especially preferred compound within the I Group is the compound, prodrugs thereof and pharmaceutically acceptable salts of said compound and prodrugs, wherein Ar is pyrid-3-yl; Z is carboxy; M is 6-(1,4-benzodioxan); and Q is —CH₂—X—CH₂— wherein X is metaphenylene.

Another especially preferred compound within the I Group is the compound wherein Ar is pyrid-3-yl; Z is carboxy; M is 2-benzofuryl; and Q is —CH₂—X—CH₂— wherein X is metaphenylene.

Another especially preferred group of compounds within the I Group, designated the J Group, comprises those compounds, prodrugs thereof and pharmaceutically acceptable salts of said compounds and said prodrugs, wherein Ar is phenyl, pyridyl or imidazolyl, said phenyl, pyridyl and imidazolyl optionally substituted independently with chloro, fluoro, methyl, methoxy, difluoromethoxy, trifluoromethyl or trifluoromethoxy; Ar³ is phenyl substituted with R³¹, wherein R³¹ is (C₁-C₇)alkyl, mono-N- or di-N,N-(C₁-C₄)alkylamine, or (C₁-C₅)alkoxy, said (C₁-C₇)alkyl or (C₁-C₅)alkoxy optionally mono-, di- or tri-substituted independently with hydroxy or fluoro; and Ar³ is further optionally mono- or di-substituted with chloro, fluoro, methyl, methoxy, difluoromethoxy, trifluoromethoxy or trifluoromethyl.

A preferred group of compounds within the J Group, designated the K Group, comprises those compounds, prodrugs thereof and pharmaceutically acceptable salts of said compounds and said prodrugs, wherein Q is —(C₅-C₇)alkylene-.

Another preferred group of compounds within the J Group, designated the L Group, comprises those compounds, prodrugs thereof and pharmaceutically acceptable salts of said compounds and said prodrugs, wherein Q is —CH₂—X—CH₂— and X is phenyl optionally mono-, di- or tri-substituted with chloro, fluoro, methoxy, difluoromethoxy, trifluoromethoxy, trifluoromethyl or methyl.

An especially preferred group of compounds within the L Group are (3-(((4-butyl-benzyl)-(pyridine-3-sulfonyl)-amino)-methyl)-phenyl)-acetic acid; (3-((benzenesulfonyl-(4-butyl-benzyl)-amino)-methyl)-phenyl)-acetic acid; (3-(((4-butyl-benzyl)-(1-methyl-1H-imidazole-4-sulfonyl)-amino)-methyl)-phenyl)-acetic acid; and (3-(((4-dimethylamino-benzyl)-(pyridine-3-sulfonyl)-amino)-methyl)-phenyl)-acetic acid.

An especially preferred compound within the L Group is the compound, prodrugs thereof and pharmaceutically acceptable salts of said compounds and said prodrugs, wherein Ar is pyrid-3-yl; Z is carboxy; M is phenyl substituted at the 4-position with n-butyl; and Q is —CH₂—X—CH₂— wherein X is metaphenylene.

Another especially preferred compound within the L Group is the compound, prodrugs thereof and pharmaceutically acceptable salts of said compounds and said prodrugs, wherein Ar is phenyl; Z is carboxy; M is phenyl substituted at the 4-position with n-butyl; and Q is —CH₂—X—CH₂— wherein X is metaphenylene.

Yet another especially preferred compound within the L Group is the compound, prodrugs thereof and pharmaceutically acceptable salts of said compounds and said prodrugs, wherein Ar is 4-(1-methyl-imidazolyi); Z is carboxy; M is phenyl substituted at the 4-position with n-butyl; and Q is —CH₂—X—CH₂— wherein X is metaphenylene.

Yet another especially preferred compound within the L Group is the compound, prodrugs thereof and pharmaceutically acceptable salts of said compounds and said prodrugs, wherein Ar is pyrid-3-yl; Z is carboxy; M is phenyl substituted at the 4-position with dimethylamino; and Q is —CH₂—X—CH₂— wherein X is metaphenylene.

Another preferred group of compounds within the J Group comprises those compounds, prodrugs thereof and pharmaceutically acceptable salts of said compounds and said prodrugs, wherein Q is —(C₂-C₄)alkylene-thienyl, —(C₂-C₄)alkylene-furanyl or —(C₂-C₄)alkylene-thiazolyl.

A preferred group of compounds within the J Group, designated the M Group, comprises those compounds, prodrugs thereof and pharmaceutically acceptable salts of said compounds and said prodrugs, wherein Q is —(C₁-C₂)—X—O—(C₁-C₂)alkylene- and X is metaphenylene, said X being optionally mono-, di- or tri-substituted with chloro, fluoro, methoxy, difluoromethoxy, trifluoromethoxy, trifluoromethyl or methyl.

An especially preferred group of compounds within the M Group are (3-(((4-dimethylamino-benzyl)-(pyridine-3-sulfonyl)-amino)-methyl)-phenoxy)-acetic acid and (3-(((4-tert-butyl-benzyl)-(pyridine-3-sulfonyl)-amino)-methyl)-phenoxy)-acetic acid.

An especially preferred compound within the M Group is the compound, prodrugs thereof and pharmaceutically acceptable salts of said compounds and said prodrugs, wherein Ar is pyrid-3-yl; Z is carboxy; M is phenyl substituted at the 4-position with dimethylamino; and Q is —CH₂—X—O—CH₂— wherein X is metaphenylene.

Another especially preferred compound within the M Group is the compound, prodrugs thereof and pharmaceutically acceptable salts of said compounds and said prodrugs, wherein Ar is pyrid-3-yl; Z is carboxy; M is phenyl substituted at the 4-position with tert-butyl; and Q is —CH₂—X—O—CH₂— wherein X is metaphenylene.

Another preferred group of compounds within the FA Group, designated the N Group, comprises those compounds, prodrugs thereof and pharmaceutically acceptable salts of said compounds and said prodrugs, wherein G is Ar; K is (C₂-C₄) alkylene or n-propenylene; Ar is phenyl, pyrazolyl, pyridazinyl, pyrazinyl, pyridyl, imidazolyl, pyrimidyl, thienyl or thiazolyl, wherein Ar is optionally mono-, di- or tri-substituted with R³, R⁴ or R⁵; and M is Ar³, optionally mono-, di- or tri-substituted with chloro, fluoro, methyl, methoxy, difluoromethoxy, trifluoromethoxy and trifluoromethyl.

An especially preferred compound within the N Group is trans-(3-(((3-(3,5-dichloro-phenyl)-allyl)-(pyridine-3-sulfonyl)-amino)-methyl)-phenyl)-acetic acid.

An especially preferred compound within the N Group is the compound, prodrugs thereof and pharmaceutically acceptable salts of said compounds and said prodrugs, wherein K is trans-n-propenylene, said M group being attached to the 1-position of the n-propenylene and said N atom being attached to the 3-position of the n-propenylene; Ar is pyrid-3-yl; M is phenyl 3,5-disubstituted with chloro; Z is carboxy; and Q is CH₂—X—CH₂— wherein X is metaphenylene.

A preferred group of compounds within the N Group, designated the O Group, comprises those compounds, prodrugs thereof and pharmaceutically acceptable salts of said compounds and said prodrugs, wherein Ar³ is phenyl optionally substituted with chloro, fluoro, methyl, methoxy, difluoromethoxy, trifluoromethoxy or trifluoromethyl.

A preferred group of compounds within the O Group, designated the P Group, comprises those compounds, prodrugs thereof and pharmaceutically acceptable salts of said compounds and said prodrugs, wherein Q is —(C₅-C₇)alkylene-.

Another group of compounds within the O Group, designated the Q Group, comprises those compounds, prodrugs thereof and pharmaceutically acceptable salts of said compounds and said prodrugs, wherein Q is —CH₂—X—CH₂— and X is metaphenylene.

Yet another group of compounds within the 0 Group, designated the R Group, comprises those compounds, prodrugs thereof and pharmaceutically acceptable salts of said compounds and said prodrugs, wherein Q is —(C₂-C₄)alkylene-X— and X is furanyl, thienyl or thiazolyl.

Yet another preferred group of compounds within the O Group, designated the S Group, comprises those compounds, prodrugs thereof and pharmaceutically acceptable salts of said compounds and said prodrugs, wherein Q is —(C₁-C₂)—X—O—(C₁-C₂)alkylene- and X is metaphenylene.

Another preferred group of compounds within the FA Group, designated the T Group, comprises those compounds, prodrugs thereof and pharmaceutically acceptable salts of said compounds and said prodrugs, wherein G is Ar; K is thioethylene or oxyethylene, Ar is phenyl, pyrazolyl, pyridazinyl, pyrazinyl, pyridyl, imidazolyl, pyrimidyl, thienyl or thiazolyl, wherein Ar is optionally substituted with up to three R³, R⁴ or R⁵; and M is Ar³, optionally mono-, di- or tri-substituted with chloro, fluoro, methyl, difluoromethoxy, trifluoromethoxy or trifluoromethyl.

A preferred group of compounds within the T Group, designated the U Group, comprises those compounds, prodrugs thereof and pharmaceutically acceptable salts of said compounds and said prodrugs, wherein Ar³ is phenyl.

A preferred group of compounds within the U Group, designated the V Group, comprises those compounds, prodrugs thereof and pharmaceutically acceptable salts of said compounds and said prodrugs, wherein Q is —(C₅-C₇)alkylene-.

Another preferred group of compounds within the U Group, designated the W Group, comprises those compounds, prodrugs thereof and pharmaceutically acceptable salts of said compounds and said prodrugs, wherein Q is —CH₂—X—CH₂— and X is metaphenylene.

Another preferred group of compounds within the U Group, designated the X Group, comprises those compounds, prodrugs thereof and pharmaceutically acceptable salts of said compounds and said prodrugs, wherein Q is —(C₂-C₄)alkylene-X— and X is furanyl, thienyl or thiazolyl.

Another preferred group of compounds within the U Group, designated the Y Group, comprises those compounds, prodrugs thereof and pharmaceutically acceptable salts of said compounds and said prodrugs, wherein Q is —(C₁-C₂)—X—O—(C₁-C₂)alkylene- and X is metaphenylene.

An especially preferred compound within the Y Group is (3-(((2-(3,5-dichloro-phenoxy)-ethyl)-(pyridine-3-sulfonyl)-amino)-methyl)-phenoxy)-acetic acid.

An especially preferred compound within the Y Group is the compound, prodrugs thereof and pharmaceutically acceptable salts of said compounds and said prodrugs, wherein K is ethylenyloxy; said M group being attached to the oxygen atom of the ethylenyloxy group and said N atom being attached to the 2-position of the ethylenyloxy group; Ar is pyrid-3-yl; M is phenyl 3,5-disubstituted with chloro; Z is carboxy and Q is —CH₂—X—O—CH₂— wherein X is a second phenyl ring and said CH₂ and OCH₂ substituents are situated in a meta substitution pattern on said second phenyl ring.

Another preferred group of compounds, designated the Z Group, comprises those compounds of Formula AA, prodrugs thereof and pharmaceutically acceptable salts of said compounds and said prodrugs, wherein B is CH.

A preferred group of compounds within the Z Group comprises those compounds, prodrugs thereof and pharmaceutically acceptable salts of said compounds and said prodrugs, wherein A is CO; G is Ar, K is methylenyl, propylenyl, propenylenyl or oxyethylenyl; M is Ar³ or Ar⁴—Ar⁵; Ar³ is phenyl or pyridyl; Ar⁴ is phenyl, thienyl, pyridyl or furanyl; Ar⁵ is (C₅-C₇)cycloalkyl, phenyl, pyridyl, imidazolyl, pyrimidyl, thienyl, pyridazinyl, pyrazinyl, imidazolyl, pyrazolyl or thiazolyl; Ar is phenyl, pyrazolyl, pyridazinyl, pyrazinyl, pyridyl, imidazolyl, pyrimidyl, thienyl or thiazolyl, wherein Ar, Ar³, Ar⁴ and Ar⁵ are optionally substituted independently with up to three chloro, fluoro, methyl, difluoromethoxy, trifluoromethoxy or trifluoromethyl.

Another especially preferred group of compounds within the Z Group comprises those compounds, prodrugs thereof and pharmaceutically acceptable salts of said compounds and said prodrugs, wherein A is CO; G is Ar, K is methylenyl, propylenyl, propenylenyl or oxyethylenyl; M is Ar³ or Ar⁴—Ar⁵; Ar³ is phenyl or pyridyl; Ar⁴ is phenyl, thienyl, pyridyl or furanyl; Ar⁵ is (C₅-C₇)cycloalkyl, phenyl, pyridyl, imidazolyl, pyrimidyl, thienyl, pyridazinyl, pyrazinyl, imidazolyl, pyrazolyl or thiazolyl; Ar is phenyl, pyrazolyl, pyridazinyl, pyrazinyl, pyridyl, imidazolyl, pyrimidyl, thienyl or thiazolyl, wherein Ar, Ar³, Ar⁴ and Ar⁵ are optionally substituted independently with up to three chloro, fluoro, methyl, difluoromethoxy, trifluoromethoxy or trifluoromethyl. Exemplary five to six membered aromatic rings optionally having one or two heteroatoms selected independently from oxygen, nitrogen and sulfur (i.e., X rings) are phenyl, furyl, thienyl, pyrrolyl, oxazolyl, thiazolyl, imidazolyl, pyrazolyl, isoxazolyl, isothiazolyl, pyridyl, pyridiazinyl, pyrimidinyl and pyrazinyl.

Exemplary partially saturated, fully saturated or fully unsaturated five to eight membered rings optionally having one to four heteroatoms selected independently from oxygen, sulfur and nitrogen (i.e., Ar, Ar¹ and Ar²) are cyclopentyl, cyclohexyl, cycloheptyl, cyclooctyl and phenyl. Further exemplary five membered rings are furyl, thienyl, 2H-pyrrolyl, 3H-pyrrolyl, pyrrolyl, 2-pyrrolinyl, 3-pyrrolinyl, pyrrolidinyl, 1,3-dioxolanyl, oxazolyl, thiazolyl, imidazolyl, 2H-imidazolyl, 2-imidazolinyl, imidazolidinyl, pyrazolyl, 2-pyrazolinyl, pyrazolidinyl, isoxazolyl, isothiazolyl, 1,2-dithiolyl, 1,3-dithiolyl, 3H-1,2-oxathiolyl, 1,2,3-oxadiazolyl, 1,2,4-oxadiazolyl, 1,2,5-oxadiazolyl, 1,3,4-oxadiazolyl, 1,2,3-triazolyl, 1,2,4-triazolyl, 1,3,4-thiadiazolyl, 1,2,3,4-oxatriazolyl, 1,2,3,5-oxatriazolyl, 3H-1,2,3-dioxazolyl, 1,2,4-dioxazolyl, 1,3,2-dioxazolyl, 1,3,4-dioxazolyl, 5H-1,2,5-oxathiazolyl and 1,3-oxathiolyl.

Further exemplary six membered rings are 2H-pyranyl, 4H-pyranyl, pyridyl; piperidinyl, 1,2-dioxinyl, 1,3-dioxinyl, 1,4-dioxanyl, morpholinyl, 1,4-dithianyl, thiomorpholinyl, pyridazinyl, pyrimidinyl, pyrazinyl, piperazinyl, 1,3,5-triazinyl, 1,2,4-triazinyl, 1,2,3-triazinyl, 1,3,5-trithianyl, 4H-1,2-oxazinyl, 2H-1,3-oxazinyl, 6H-1,3-oxazinyl, 6H-1,2-oxazinyl, 1,4-oxazinyl, 2H-1,2-oxazinyl, 4H-1,4-oxazinyl, 1,2,5-oxathiazinyl, 1,4-oxazinyl, o-isoxazinyl, p-isoxazinyl, 1,2,5-oxathiazinyl, 1,2,6-oxathiazinyl, 1,4,2-oxadiazinyl and 1,3,5,2-oxadiazinyl.

Further exemplary seven membered rings are azepinyl, oxepinyl, thiepinyl and 1,2,4-diazepinyl.

Further exemplary eight membered rings are cyclooctyl, cyclooctenyl and cyclooctadienyl.

Exemplary bicyclic rings consisting of two fused independently partially saturated, fully saturated or fully unsaturated five and/or six membered rings, taken independently, optionally having one to four heteroatoms selected independently from nitrogen, sulfur and oxygen are indolizinyl, indolyl, isoindolyl, 3H-indolyl, 1H-isoindolyl, indolinyl, cyclopenta(b)pyridinyl, pyrano(3,4-b)pyrrolyl, benzofuryl, isobenzofuryl, benzo(b)thienyl, benzo(c)thienyl, 1H-indazolyl, indoxazinyl, benzoxazolyl, anthranilyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2H-1-benzopyranyl, 1,4-benzodioxan, pyrido(3,4-b)-pyridinyl, pyrido(3,2-b)-pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl.

Exemplary tricyclic rings consisting of three fused independently partially saturated, fully saturated or fully unsaturated five and/or six membered rings, taken independently, optionally having one to four heteroatoms selected independently from nitrogen, sulfur and oxygen are indacenyl, biphenylenyl, acenaphthylenyl, fluorenyl, phenalenyl, phenanthrenyl, anthracenyl, naphthothienyl, thianthrenyl, xanthenyl, phenoxathiinyl, carbazolyl, carbolinyl, phenanthridinyl, acridinyl, perimidinyl, phenanthrolinyl, phenazinyl, phenothiazinyl and phenoxazinyl. It will be understood that the fully saturated and all partially unsaturated forms of these rings are within the scope of this invention. Further, it will be understood that nitrogen may be substituted as the heteroatom at any position, including a bridgeghead position, in the heterocyclic rings. Further still, it will be understood that sulfur and oxygen may be substituted as the heteroatom at any non-bridgehead position within the heterocyclic rings.

By alkylene is meant saturated hydrocarbon (straight chain or branched) wherein a hydrogen atom is removed from each of the terminal carbons. Exemplary of such groups (assuming the designated length encompases the particular example) are methylene, ethylene, propylene, butylene, pentylene, hexylene and heptylene.

By alkenylene is meant a hydrocarbon containing monounsaturation in the form of one double bond wherein said hydrocarbon is straight chain or branched and wherein a hydrogen atom is removed from each of the terminal carbons. Exemplary of such groups (assuming the designated length encompasses the particular example) are ethenylene (or vinylene), propenylene, butenylene, pentenylene, hexenylene and heptenylene.

By alkynylene is meant a hydrocarbon containing di-unsaturation in the form of one triple bond wherein said hydrocarbon is straight chain or branched and wherein a hydrogen atom is removed from each of the terminal carbons. Exemplary of such groups (assuming the designated length encompasses the particular example) are ethynylene, propynylene, butynylene, pentynylene, hexynylene and heptynylene.

By halo is meant chloro, bromo, iodo, or fluoro.

By alkyl is meant straight chain saturated hydrocarbon or branched saturated hydrocarbon. Exemplary of such alkyl groups (assuming the designated length encompasses the particular example) are methyl, ethyl, propyl, isopropyl, butyl, sec-butyl, tertiary butyl, pentyl, isopentyl, neopentyl, tertiary pentyl, 1-methylbutyl, 2-methylbutyl, 3-methylbutyl, hexyl, isohexyl, heptyl and octyl.

By alkoxy is meant straight chain saturated alkyl or branched saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

As used herein, the term mono-N- or di-N,N-(C₁-Cₓ)alkyl . . . refers to the (C₁-Cₓ)alkyl moiety taken independently when it is di-N,N-(C₁-Cₓ)alkyl . . . (x refers to integers and is taken independently when two (C₁-Cₓ)alkyl groups are present, e.g., methylethylamino is within the scope of di-N,N-(C₁-Cₓ)alkyl).

Unless otherwise stated the “M” moieties defined above are optionally substituted (e.g., the mere listing of a substituent such as R¹ in a subgenus or dependent claim does not mean that M is always substituted with the R¹ moiety unless it is stated that the M moiety is substituted with R¹). However, in the compounds of Formula AA, when K is a bond and M is phenyl, said phenyl group is substituted with one to three substituents. Additionally, in the compounds of Formula AA, when Ar or Ar¹ is a fully saturated five to eight membered ring, said ring is unsubstituted.

It is to be understood that if a carbocyclic or heterocyclic moiety may be bonded or otherwise attached to a designated substrate, through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth.

A particularly preferred compound of Formula AA is (3-(((4-tert-butyl-benzyl)-(pyridine-3-sulfonyl)-amino)-methyl)-phenoxy)-acetic acid, or a pharmaceitcally acceptable salt or prodrug thereof, or a salt of a prodrug. A particularly preferred salt is the sodium salt.

Other EP₂ selective receptor agonists that can be used in the present invention include the prostaglandin receptor agonists disclosed in U.S. Pat. Nos. 6,288,120; and 6,124,314; and PCT published patent application WO 98/58911 (PCT/IB98/00866). A preferred EP₂ compound disclosed in U.S. Pat. No. 6,288,120 is 7-[(4-butyl-benzyl)-methanesulfonyl-amino]-heptanoic acid or a pharmaceutically acceptable salt or prodrug thereof, or a salt of a prodrug. A preferred salt of 7-[(4-butyl-benzyl)-methanesulfonyl-amino]-heptanoic acid is the monosodium salt.

Other EP₂ selective receptor agonists that can be used in the present invention include the compounds disclosed in the following: Burk, Robert M.; Holoboski, Mark; Posner, Mari F., Preparation of prostaglandin E2 analogs as EP2-receptor agonists-US patent application no. 2002187961; Burk, Robert M.; Holoboski, Mark; Posner, Mari F., Preparation of prostaglandin E2 analogs as EP2-receptor agonists—U.S. Pat. No. 6,376,533; Duckworth, N.; Marshall, K.; Clayton, J. K., An investigation of the effect of the prostaglandin EP2 receptor agonist, butaprost, on the human isolated myometrium from pregnant and non-pregnant women, Journal of Endocrinology (2002), 172(2), 263-269; Tani, Kousuke; Naganawa, Atsushi; Ishida, Akiharu; Egashira, Hiromu; Odagaki, Yoshihiko; Miyazaki, Toru; Hasegawa, Tomoyuki; Kawanaka, Yasufumi; Nakai, Hisao; Ohuchida, Shuichi; Toda, Masaaki. Synthesis of a highly selective EP2-receptor agonist, Synlett (2002), (2), 239-242; Tani, Kousuke; Naganawa, Atsushi; Ishida, Akiharu; Egashira, Hiromu; Sagawa, Kenji; Harada, Hiroyuki; Ogawa, Mikio; Maruyama, Takayuki; Ohuchida, Shuichi; Nakai, Hisao; Kondo, Kigen; Toda, Masaaki. Development of a highly selective EP2-receptor agonist. Part 2. Identification of 16-Hydroxy-17,17-trimethylene 9b-chloro PGF derivatives, Bioorganic & Medicinal Chemistry (2002), 10(4), 1107-1114; Tani, Kousuke; Naganawa, Atsushi; Ishida, Akiharu; Sagawa, Kenji; Harada, Hiroyuki; Ogawa, Mikio; Maruyama, Takayuki; Ohuchida, Shuichi; Nakai, Hisao; Kondo, Kigen; Toda, Masaaki, Development of a highly selective EP2-receptor agonist. Part 1. Identification of 16-hydroxy-17,17-trimethylene PGE2 derivatives, Bioorganic & Medicinal Chemistry (2002), 10(4), 1093-1106; Michelet, Jean-Francois; Mahe, Yann; Bernard, Bruno, Use of non-prostanoic agonists of EP-2 and/or EP4 prostaglandin receptors as cosmetic agent for reducing or stopping hair loss—European patent application EP 1175891 A1; Tani, K.; Naganawa, A.; Ishida, A.; Egashira, H.; Sagawa, K.; Harada, H.; Ogawa, M.; Maruyama, T.; Ohuchida, S.; Nakai, H.; Kondo, K.; Toda, M., Design and Synthesis of a Highly Selective EP2-Receptor Agonist, Bioorganic & Medicinal Chemistry Letters (2001), 11(15), 2025-2028; Crider, J. Y.; Sharif, N. A., Functional pharmacological evidence for EP2 and EP4 prostanoid receptors in immortalized human trabecular meshwork and non-pigmented ciliary epithelial cells, International Journal of Environmental Studies (2000), 58(1), 35-46; Crider, J. Y.; Sharif, N., A. Functional pharmacological evidence for EP2 and EP4 prostanoid receptors in immortalized human trabecular meshwork and nonpigmented ciliary epithelial cells. Journal of Ocular Pharmacology and Therapeutics (2001), 17(1), 35-46; Klimko, Peter G.; Sharif, Najam A.; Griffin, Brenda W. Prostaglandin E agonists for treatment of glaucoma—WO 0038667 A2; Woodward, David F., EP2 receptor agonists as neuroprotective agents for the eye—U.S. Pat. No. 5,877,211; Regan, John W.; Gil, Daniel W.; Woodward, David F., Cloning of a novel human prostaglandin receptor with characteristics of the pharmacologically defined EP2 subtype—U.S. Pat. No. 5,716,835; Woodward, David F. EP2-receptor agonists as agents for lowering intraocular pressure—U.S. Pat. No. 5,698,598; Woodward, David F. EP2-receptor agonists as agents for lowering intraocular pressure.—WO 9519964; Woodward, D. F.; Bogardus, A. M.; Donello, J. E.; Fairbairn, C. E.; Gil, D. W.; Kedzie, K. M.; Burke, J. A.; Kharlamb, A.; Runde, E.; et al., Molecular characterization and ocular hypotensive properties of the prostanoid EP2 receptor, Journal of Ocular Pharmacology and Therapeutics (1995), 11 (3), 447-54; Nials, Anthony T.; Vardey, Christopher J.; Denyer, Lois H.; Thomas, Malcolm; Sparrow, Susan J.; Shepherd, Gillian D.; Coleman, Robert A., AH13205, a selective prostanoid EP2-receptor agonist, Cardiovascular Drug Reviews (1993), 11(2), 165-79; and Woodward, D. F.; Protzman, C. E.; Krauss, A. H. P.; Williams, L. S., Identification of 19(R)—OH prostaglandin E2 as a selective prostanoid EP2-receptor agonist, Prostaglandins (1993), 46(4), 371-83.

Other EP₂ agonist compounds that can be used in the present invention include those compounds of Formula BB below, which are disclosed in U.S. Pat. No. 6,288,120, issued Sep. 11, 2001. 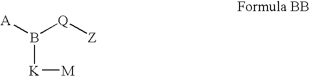
or a pharmaceutically-acceptable salt or prodrug thereof
wherein
either (i):
B is N;
A is (C₁-C₆)alkylsulfonyl, (C₃-C₇)cycloalkylsulfonyl, (C₃-C₇)cycloalkyl(C₁-C₆)alkylsulfonyl, said A moieties optionally mono-, di- or tri-substituted on carbon independently with hydroxy, (C₁-C₄)alkyl or halo;
Q is
   (C₂-C₆)alkylene-W—(C₁-C₃)alkylene-,
   (C₃-C₈)alkylene-, said —(C₃-C₈)alkylene- optionally substituted with up to four substituents independently selected from fluoro or (C₁-C₄)alkyl,
   X—(C₁-C₅)alkylene-,
   (C₁-C₅)alkylene-X—,
   (C₁-C₃)alkylene-X—(C₁-C₃)alkylene-,
   (C₂-C₄)alkylene-W—X—(CO—C₃)alkylene-,
   (C₀-C₄)alkylene-X—W—(C₁-C₃)alkylene-,
   (C₂-C₅)alkylene-W—X—W—(C₁-C₃)alkylene-, wherein the two occurrences of W are independent of each other,
   (C₁-C₄)alkylene-ethenylene-(C₁-C₄)alkylene-,
   (C₁-C₄)alkylene-ethenylene-(CO—C₂)alkylene-X—(CO—C₅)alkylene-,
   (C₁-C₄)alkylene-ethenylene-(CO—C₂)alkylene-X—W—(C₁-C₃)alkylene-,
   (C₁-C₄)alkylene-ethynylene-(C₁-C₄)alkylene-, or
   (C₁-C₄)alkylene-ethynylene-X—(C₀-C₃)alkylene-;
W is oxy, thio, sulfino, sulfonyl, aminosulfonyl-, -mono-N-(C₁-C₄)alkyleneaminosulfonyl-, sulfonylamino, N-(C₁-C₄)alkylenesulfonylamino, carboxamido, N-(C₁-C₄)alkylenecarboxamido, carboxamidooxy, N-(C₁-C₄)alkylenecarboxamidooxy, carbamoyl, -mono-N-(C₁-C₄)alkylenecarbamoyl, carbamoyloxy, or -mono-N-(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen, and sulfur; said ring optionally mono-, or di-substituted independently with halo, (C₁-C₃)alkyl, trifluoromethyl, trifluoromethyloxy, difluoromethyloxy, hydroxyl, (C₁-C₄)alkoxy, or carbamoyl;
Z is carboxyl, (C₁-C₆)alkoxycarbonyl, tetrazolyl, 1,2,4-oxadiazolyl, 5-oxo-1,2,4-oxadiazolyl, (C₁-C₄)alkylsulfonylcarbamoyl or phenylsulfonylcarbamoyl;
K is a bond, (C₁-C₈)alkylene, thio(C₁-C₄)alkylene or oxy(C₁-C₄)alkylene, said (C₁-C₈)alkylene optionally mono-unsaturated and wherein K is optionally mono-, di- or tri-substituted independently with fluoro, methyl or chloro;
M is —Ar, —Ar¹—V—Ar², —Ar¹—S—Ar² or —Ar¹—O—Ar² wherein Ar, Ar¹ and Ar² are each independently a partially saturated, fully saturated or fully unsaturated five to eight membered ring optionally having one to four heteroatoms selected independently from oxygen, sulfur and nitrogen, or, a bicyclic ring consisting of two fused partially saturated, fully saturated or fully unsaturated five or six membered rings, taken independently, optionally having one to four heteroatoms selected independently from nitrogen, sulfur and oxygen;
said Ar, Ar¹ and Ar² moieties optionally substituted, on one ring if the moiety is monocyclic, or one or both rings if the moiety is bicyclic, on carbon with up to three substituents independently selected from R¹, R² and R³ wherein R¹, R² and R³ are hydroxy, nitro, halo, (C₁-C₆)alkoxy, (C₁-C₄)alkoxy(C₁-C₄)alkyl, (C₁-C₄)alkoxycarbonyl, (C₁-C₇)alkyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl or mono-N- or di-N,N-(C₁-C₄)alkylaminosulfinyl;
R¹, R² and R³ are optionally mono-, di- or tri-substituted on carbon independently with halo or hydroxy; and
V is a bond or (C₁-C₃)alkylene optionally mono- or di-substituted independently with hydroxy or fluoro with the proviso that when K is (C₂-C₄)alkylene and M is Ar and Ar is cyclopent-1-yl, cyclohex-1-yl, cyclohept-1-yl or cyclooct-1-yl then said (C₅-C₈)cycloalkyl substituents are not substituted at the one position with hydroxy;
or (ii):
B is N;
A is (C₁-C₆)alkanoyl, or (C₃-C₇)cycloalkyl(C₁-C₆)alkanoyl, said A moieties optionally mono-, di- or tri-substituted independently on carbon with hydroxy or halo;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₂-C₅)alkylene-,
   (C₁-C₅)alkylene-X—,
   (C₁-C₃)alkylene-X—(C₁-C₃)alkylene-,
   (C₂-C₄)alkylene-W—X—(CO—C₃)alkylene-,
   (C₀-C₄)alkylene-X—W—(C₁-C₃)alkylene-,
   (C₂-C₅)alkylene-W—X—W—(C₁-C₃)alkylene-, wherein the two occurrences of W are independent of each other,
   (C₁-C₄)alkylene-ethenylene-(C₁-C₄)alkylene-,
   (C₁-C₄)alkylene-ethenylene-(C₀-C₂)alkylene-X—(C₀-C₅)alkylene-,
   (C₁-C₄)alkylene-ethenylene-(C₀-C₂)alkylene-X—W—(C₁-C₃)alkylene-,
   (C₁-C₄)alkylene-ethynylene-(C₁-C₄)alkylene-, or
   (C₁-C₄)alkylene-ethynylene-X—(C₀-C₃)alkylene-;
W is oxy, thio, sulfino, sulfonyl, aminosulfonyl-, -mono-N-(C₁-C₄)alkyleneaminosulfonyl-, sulfonylamino, N-(C₁-C₄)alkylenesulfonylamino, carboxamido, N-(C₁-C₄)alkylenecarboxamido, carboxamidooxy, N-(C₁-C₄)alkylenecarboxamidooxy, carbamoyl, -mono-N-(C₁-C₄)alkylenecarbamoyl, carbamoyloxy, or -mono-N-(C₁-C₄)alkylenecarbamoyloxy, wherein said W alkyl groups are optionally substituted on carbon with one to three fluorines;
X is a five or six membered aromatic ring optionally having one or two heteroatoms independently selected from oxygen, nitrogen, and sulfur; said ring optionally mono-, or di-substituted independently with halo, (C₁-C₃)alkyl, trifluoromethyl, trifluoromethyloxy, difluoromethyloxy, hydroxyl, (C₁-C₄)alkoxy, or carbamoyl;
Z is carboxyl, (C₁-C₆)alkoxycarbonyl, tetrazolyl, 1,2,4-oxadiazolyl, 5-oxo-1,2,4-oxadiazolyl, (C₁-C₄)alkylsulfonylcarbamoyl or phenylsulfonylcarbamoyl;
K is (C₁-C₈)alkylene, thio(C₁-C₄)alkylene or oxy(C₁-C₄)alkylene, said (C₁-C₈)alkylene optionally mono-unsaturated and wherein K is optionally mono-, di- or tri-substituted independently with fluoro, methyl or chloro;
M is —Ar, —Ar¹—V—Ar², —Ar¹—S—Ar² or —Ar¹—O—Ar² wherein Ar, Ar¹ and Ar² are each independently a partially saturated, fully saturated or fully unsaturated five to eight membered ring optionally having one to four heteroatoms selected independently from oxygen, sulfur and nitrogen, or, a bicyclic ring consisting of two fused partially saturated, fully saturated or fully unsaturated five or six membered rings, taken independently, optionally having one to four heteroatoms selected independently from nitrogen, sulfur and oxygen;
said Ar, Ar¹ and Ar² moieties optionally substituted, on one ring if the moiety is monocyclic, or one or both rings if the moiety is bicyclic, on carbon with up to three substituents independently selected from R¹, R² and R³ wherein R¹, R² and R³ are H, hydroxy, nitro, halo, (C₁-C₆)alkoxy, (C₁-C₄)alkoxy(C₁-C₄)alkyl, (C₁-C₄)alkoxycarbonyl, (C₁-C₇)alkyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl or mono-N- or di-N,N-(C₁-C₄)alkylaminosulfinyl;
R¹, R² and R³ are optionally mono-, di- or tri-substituted on carbon independently with halo or hydroxy; and
V is a bond or (C₁-C₃)alkylene optionally mono- or di-substituted independently with hydroxy or fluoro
with the proviso that when K is (C₂-C₄)alkylene and M is Ar and Ar is cyclopent-1-yl, cyclohex-1-yl, cyclohept-1-yl or cycloct-1-yl then said (C₅-C₈)cycloalkyl substituents are not substituted at the one position with hydroxy
and with the proviso that 6-[(3-Phenyl-propyl)-(2-propyl-pentanoyl)-amino]-hexanoic acid and its ethyl ester are not included
or (iii):

B is C(H);
A is (C₁-C₆)alkanoyl, or (C₃-C₇)cycloalkyl(C₁-C₆)alkanoyl, said A moieties optionally mono-, di- or tri-substituted on carbon independently with hydroxy or halo;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₁-C₅)alkylene-,
   (C₁-C₅)alkylene-X—,
   (C₁-C₃)alkylene-X—(C₁-C₃)alkylene-,
   (C₂-C₄)alkylene-W—X—(CO—C₃)alkylene-,
   (C₀-C₄)alkylene-X—W—(C₁-C₃)alkylene-,
   (C₂-C₅)alkylene-W—X—W—(C₁-C₃)alkylene-, wherein the two occurrences of W are independent of each other,
   (C₁-C₄)alkylene-ethenylene-(C₁-C₄)alkylene-,
   (C₁-C₄)alkylene-ethenylene-(CO—C₂)alkylene-X—(C₀-C₃)alkylene-,
   (C₁-C₄)alkylene-ethenylene-(C₀-C₂)alkylene-X—W—(C₁-C₃)alkylene-,
   (C₁-C₄)alkylene-ethynylene-(C₁-C₄)alkylene-, or
   (C₁-C₄)alkylene-ethynylene-X—(C₀-C₃)alkylene-;
W is oxy, thio, sulfino, sulfonyl, aminosulfonyl-, -mono-N-(C₁-C₄)alkyleneaminosulfonyl-, sulfonylamino, N-(C₁-C₄)alkylenesulfonylamino, carboxamido, N-(C₁-C₄)alkylenecarboxamido, carboxamidooxy, N-(C₁-C₄)alkylenecarboxamidooxy, carbamoyl, -mono-N-(C₁-C₄)alkylenecarbamoyl, carbamoyloxy, or -mono-N-(C₁-C₄)alkylenecarbamoyloxy, wherein said W alkyl groups are optionally substituted on carbon with one to three fluorines;
X is a five or six membered aromatic ring optionally having one or two heteroatoms selected independently from oxygen, nitrogen and sulfur; said ring optionally mono-, or di-substituted independently with halo, (C₁-C₃)alkyl, trifluoromethyl, trifluoromethyloxy, difluoromethyloxy, hydroxyl, (C₁-C₄)alkoxy, or carbamoyl;
Z is carboxyl, (C₁-C₆)alkoxycarbonyl, tetrazolyl, 1,2,4-oxadiazolyl, 5-oxo-1,2,4-oxadiazolyl, (C₁-C₄)alkylsulfonylcarbamoyl or phenylsulfonylcarbamoyl;
K is a bond, (C₁-C₈)alkylene, thio(C₁-C₄)alkylene, (C₄-C₇)cycloalkyl(C₁-C₆)alkylene or oxy(C₁-C₄)alkylene, said (C₁-C₈)alkylene optionally mono-unsaturated and wherein K is optionally mono-, di- or tri-substituted independently with fluoro, methyl or chloro;
M is —Ar, —Ar¹—V—Ar², —Ar¹—S—Ar² or —Ar¹—O—Ar² wherein Ar, Ar¹ and Ar² are each independently a partially saturated, fully saturated or fully unsaturated five to eight membered ring optionally having one to four heteroatoms selected independently from oxygen, sulfur and nitrogen, or, a bicyclic ring consisting of two fused partially saturated, fully saturated or fully unsaturated five or six membered rings, taken independently, optionally having one to four heteroatoms selected independently from nitrogen, sulfur and oxygen;
said Ar, Ar¹ and Ar² moieties optionally substituted, on one ring if the moiety is monocyclic, or one or both rings if the moiety is bicyclic, on carbon with up to three substituents independently selected from R¹, R² and R³ wherein R¹, R² and R³ are H, hydroxy, nitro, halo, (C₁-C₆)alkoxy, (C₁-C₄)alkoxy(C₁-C₄)alkyl, (C₁-C₄)alkoxycarbonyl, (C₁-C₇)alkyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl or mono-N- or di-N,N-(C₁-C₄)alkylaminosulfinyl;
R¹, R² and R³ are optionally mono-, di- or tri-substituted independently on carbon with halo or hydroxy; and
V is a bond or (C₁-C₃)alkylene optionally mono- or di-substituted independently with hydroxy or fluoro with the proviso that when K is (C₂-C₄)alkylene and M is Ar and Ar is cyclopent-1-yl, cyclohex-1-yl, cyclohept-1-yl or cyclooct-1-yl then said (C₅-C₈)cycloalkyl substituents are not substituted at the one position with hydroxy.

A preferred group of compounds, designated the A Group, contains those compounds having the Formula BB as shown above wherein
B is N;
A is (C₁-C₆)alkylsulfonyl, (C₃-C₆)cycloalkylsulfonyl or (C₃-C₆)cycloalkyl(C₁-C₆)alkylsulfonyl, said A moieties optionally mono-, di-, or tri-substituted on carbon with fluoro;
X is phenyl, thienyl, or thiazolyl said phenyl, thienyl or thiazolyl optionally mono- or di-substituted independently with fluoro, chloro, trifluoromethyl, methoxy, difluoromethoxy or trifluoromethoxy;
W is oxy, thio or sulfonyl;
Z is carboxyl, (C₁-C₄)alkoxycarbonyl or tetrazolyl;
K is methylene or ethylene;
Ar, Ar¹ and Ar² are each independently (C₅-C₇)cycloalkyl, phenyl, thienyl, thiazolyl, pyridyl, pyrimidyl, oxazolyl, furanyl, imidazolyl, isoxazolyl, pyrazinyl or pyrazolyl;
R¹ is halo, (C₁-C₆)alkoxy, (C₁-C₇)alkyl, (C₃-C₇)cycloalkyl, or (C₃-C₇)cycloalkyl(C₁-C₄)alkyl, said (C₁-C₆)alkoxy, (C₁-C₇)alkyl, (C₃-C₇)cycloalkyl or (C₃-C₇)cycloalkyl(C₁-C₄)alkyl, optionally mono-, di- or tri-substituted independently with hydroxy, fluoro or chloro; and

R² and R³ are chloro, fluoro, methyl, methoxy, difluoromethoxy, trifluoromethoxy or trifluoromethyl.

A group of compounds which is preferred among the A Group of compounds designated the B Group, contains those compounds wherein
A is (C₁-C₃)alkylsulfonyl;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl, —X—(C₂-C₅)alkylene-,
   —(C₁-C₅)alkylene-X—,
   (C₁-C₃)alkylene-X—(C₁-C₃)alkylene-,
   (C₂-C₄)alkylene-W—X—(C₀-C₃)alkylene-, or
   (C₀-C₄)alkylene-X—W—(C₁-C₃)alkylene-;
M is —Ar¹—V—Ar² or —Ar¹—O—Ar² wherein Ar¹ and Ar² are each independently phenyl, pyridyl or thienyl;
V is a bond or (C₁-C₂)alkylene;
R¹ is chloro, fluoro, (C₁-C₄)alkyl or (C₁-C₄)alkoxy, said (C₁-C₄)alkyl and (C₁-C₄)alkoxy optionally mono-, di- or tri-substituted independently with hydroxy or fluoro; and
R² and R³ are each independently chloro or fluoro.

Especially preferred compounds within the B Group of compounds are
7-[(2′-Hydroxymethyl-biphenyl-4-ylmethyl)-methanesulfonyl-amino]-heptanoic acid,
7-{[4-(3-Hydroxymethyl-thiophen-2-yl)-benzyl]-methanesulfonyl-amino}-heptanoic acid, and
7-[(2′-Chloro-biphenyl-4-ylmethyl)-methanesulfonyl-amino]-heptanoic acid.

Especially preferred compounds within the B Group of compounds are compounds wherein
a. A is methylsulfonyl;
   Q is n-hexylene;
   Z is carboxyl;
   K is methylene; and
   M is 4-(2-hydroxymethylphenyl)phenyl;
b. A is methylsulfonyl;
   Q is n-hexylene;
   Z is carboxyl;
   K is methylene; and

M is 4-(3-hydroxymethylthien-2-yl)phenyl; and
c. A is methylsulfonyl;
   Q is n-hexylene;
   Z is carboxyl;
   K is methylene; and
   M is 4-(2-chlorophenyl)phenyl.

A preferred group of compounds, designated the C Group, contains those compounds having the Formula BB as shown above wherein
B is N;
A is (C₁-C₆)alkylsulfonyl, (C₃-C₆)cycloalkylsulfonyl, (C₃-C₆)cycloalkyl(C₁-C₆)alkylsulfonyl;
X is phenyl, thienyl, or thiazolyl said phenyl, thienyl or thiazolyl optionally mono- or di-substituted independently with fluoro, chloro, trifluoromethyl, methoxy, difluoromethoxy or trifluoromethyloxy;
W is oxy, thio or sulfonyl;
Z is carboxyl, (C₁-C₄)alkoxycarbonyl or tetrazolyl;
K is (C₁-C₈)alkylene or oxy(C₁-C₄)alkylene, said (C₁-C₈)alkylene optionally mono-unsaturated and wherein K is optionally mono-, di- or tri-substituted independently with methyl, fluoro or chloro;
M is —Ar, said —Ar is phenyl, thienyl, pyridyl, thiazolyl, oxazolyl, isoxazolyl, naphthalenyl, benzo[b]furanyl, benzo[b]thiophenyl, indanyl, furanyl, benzo[1,3]dioxolyl, benzimidazolyl, benzisoxazolyl, 2,3-dihydrobenzo[1,4]dioxinyl, 2,3-dihydrobenzofuranyl, pyrazolyl, pyrimidyl, imidazolyl, quinolinyl, isoquinolinyl, benzoxazolyl, benzothiazolyl, indolyl, 1,2,3,4-tetrahydronaphthalenyl, cyclohexyl, cyclopentyl, cyclobutyl, cycloheptyl or chromanyl;
R¹ is halo, (C₁-C₆)alkoxy, (C₁-C₇)alkyl, (C₃-C₇)cycloalkyl, (C₁-C₇)alkanoyl or (C₃-C₇)cycloalkyl(C₁-C₄)alkyl, said (C₁-C₆)alkoxy, (C₁-C₇)alkyl, (C₃-C₇)cycloalkyl, (C₁-C₇)alkanoyl or (C₃-C₇)cycloalkyl(C₁-C₄)alkyl, optionally mono-, di- or tri-substituted independently with hydroxy, fluoro or chloro; and
R² and R³ are each independently hydroxy, halo, trifluoromethyl, (C₁-C₇)alkyl, (C₁-C₄)alkoxy, (C₁-C₅)alkanoyl, cyano, (C₃-C₇)cycloalkyl, (C₃-C₇)cycloalkyl(C₁-C₄)alkyl, formyl, difluoromethoxy, trifluoromethoxy or carbamoyl.

It is especially preferred for Group C compounds that K is not optionally mono-, di- or tri-substituted independently with methyl, fluoro or chloro.

A group of compounds which is preferred among the C Group of compounds, designated the D Group, contains those compounds wherein
K is methylene;
A is (C₁-C₃)alkylsulfonyl;
M is —Ar and —Ar is phenyl, thiazolyl, pyridyl, thienyl, oxazolyl, furanyl, cyclopentyl or cyclohexyl wherein —Ar is substituted with at least R¹;
R¹ is (C₁-C₇)alkyl or (C₁-C₅)alkoxy, said (C₁-C₇)alkyl or (C₁-C₅)alkoxy optionally mono-, di- or tri-substituted independently with hydroxy or fluoro; and

R² and R³ are each independently chloro, fluoro, methyl, difluoromethoxy, trifluoromethoxy or trifluoromethyl.

Especially preferred among the D Group of compounds are
7-{4-(1-Hydroxy-hexyl)-benzyl]-methanesulfonyl-amino]-heptanoic acid,
7-[(4-Butyl-benzyl)-methanesulfonyl-amino]-heptanoic acid,
7-{[5-(1-Hydroxy-hexyl)-thiophen-2-ylmethyl]-methanesulfonyl-amino}-heptanoic acid and
(3-{[(4-Butyl-benzyl)-methanesulfonyl-amino]-methyl}phenyl)-acetic acid.

A group of compounds which is preferred among the D Group of compounds, designated the E Group, contains those compounds wherein
Q is —(C₂-C₆)alkylene-W—(C₁-C₃)alkylene-; and
W is oxy.

A group of compounds which is preferred among the D Group of compounds, designated the F Group, contains those compounds wherein
Q is —(C₃-C₈)alkylene-, said —(C₃-C₈)alkylene- optionally substituted with from one to four fluorines.

Especially preferred compounds among the F Group of compounds are compounds wherein
a. A is methylsulfonyl;
   Q is n-hexylene;
   Z is carboxyl;
   K is methylene; and
   M is 4-(1-hydroxy-n-hexylene-1-yl)phenyl;
b. A is methylsulfonyl;
   Q is n-hexylene;
   Z is carboxyl;
   K is methylene; and
   M is 4-(n-butylene-1-yl)phenyl; and
c. A is methylsulfonyl;
   Q is n-hexylene;
   Z is carboxyl;
   K is methylene; and
   M is 5-(1-hydroxy-n-hexylene-1-yl)thien-2-yl.

A group of compounds which is preferred among the D Group of compounds, designated the G Group, contains those compounds wherein
Q is —X—(C₁-C₅)alkylene-; and
X is thienyl or phenyl; said phenyl and thienyl optionally mono- or di-substituted independently with fluoro, chloro, trifluoromethyl or methoxy.

A group of compounds which is preferred among the D Group of compounds, designated the H Group, contains those compounds wherein
Q is —(C₁-C₅)alkylene-X—; and
X is thienyl or phenyl; said phenyl and thienyl optionally mono- or di-substituted independently with fluoro, chloro, trifluoromethyl or methoxy.

A group of compounds which is preferred among the D Group of compounds, designated the I Group, contains those compounds wherein
Q is —(C₁-C₃)alkylene-X—(C₁-C₃)alkylene-; and
X is thienyl or phenyl; said phenyl and thienyl optionally mono- or di-substituted independently with fluoro, chloro, trifluoromethyl or methoxy.

An especially preferred compound within the I Group of compounds is a compound wherein
A is methylsulfonyl;
Q is 3-methylenephenylmethyl;
Z is carboxyl;
K is methylene; and
M is 4-(n-butylene-1-yl)phenyl.

A group of compounds which is preferred among the D Group of compounds, designated the J Group, contains those compounds wherein
Q is —(C₂-C₄)alkylene-W—X—(C₀-C₃)alkylene-;
X is thienyl or phenyl; said phenyl and thienyl optionally mono- or di-substituted independently with fluoro, chloro, trifluoromethyl or methoxy; and
W is oxy.

A group of compounds which is preferred among the D Group of compounds, designated the K Group, contains those compounds wherein
Q is —(C₀-C₄)alkylene-X—W—(C₁-C₃)alkylene-;
X is thienyl or phenyl; said phenyl and thienyl optionally mono- or di-substituted independently with fluoro, chloro, trifluoromethyl or methoxy; and
W is oxy.

A group of compounds which is preferred among the D Group of compounds, designated the L Group, contains those compounds wherein
Q is —(C₂-C₄)alkylene-W—X—W—(C₁-C₃)alkylene-;
W is oxy; and
X is thienyl or phenyl; said phenyl and thienyl optionally mono- or di-substituted independently with fluoro, chloro, trifluoromethyl or methoxy.

A group of compounds which is preferred among the D Group of compounds, designated the M Group, contains those compounds wherein
Q is —(C₁-C₄)alkylene-ethenylene-(C₁-C₄)alkylene-; and
M is —Ar and —Ar is phenyl, thiazolyl, pyridyl or thienyl.

A group of compounds which is preferred among the D Group of compounds, designated the N Group, contains those compounds wherein
Q is —(C₁-C₄)alkylene-ethenylene-(C₀-C₂)alkylene-X—(CO—C₃)alkylene-; and
X is thienyl or phenyl; said phenyl and thienyl optionally mono- or di-substituted independently with fluoro, chloro, trifluoromethyl or methoxy.

A group of compounds which is preferred among the D Group of compounds, designated the 0 Group, contains those compounds wherein
Q is —(C₁-C₃)alkylene-ethenylene-(C₀-C₂)alkylene-X—W—(C₁-C₃)alkylene-;
W is oxy; and
X is thienyl or phenyl; said phenyl and thienyl optionally mono- or di-substituted independently with fluoro, chloro, trifluoromethyl or methoxy.

A group of compounds which is preferred among the D Group of compounds, designated the P Group, contains those compounds wherein
Q is —(C₁-C₄)alkylene-ethynylene-(C₁-C₄)alkylene-.

A group of compounds which is preferred among the D Group of compounds designated the Q Group, contains those compounds wherein
Q is —(C₁-C₄)alkylene-ethynylene-X—(C₀-C₃)alkylene-; and
X is thienyl or phenyl; said phenyl and thienyl optionally mono- or di-substituted independently with fluoro, chloro, trifluoromethyl or methoxy.

A group of compounds which is preferred among the C Group of compounds designated the R Group, contains those compounds wherein
A is (C₁-C₃)alkylsulfonyl;
K is (C₁-C₈)alkylene;
—Ar is phenyl, thiazolyl, pyridyl, thienyl, benzofuranyl, benzo[1,3]dioxolyl, 2,3-dihydrobenzo[1,4]dioxine, 2,3-dihydrobenzofuranyl, benzimidazolyl, benzo[b]thiophenyl, cyclopentyl or cyclohexyl; and
R¹, R² and R³ are each independently hydroxy, halo, trifluoromethyl, difluoromethoxy, trifluoromethoxy, (C₁-C₄)alkoxy or (C₁-C₇)alkyl.

Preferred compounds among the R Group are
7-{[3-(3-Chloro-phenyl)-propyl]-methanesulfonyl-amino}-heptanoic acid,
7-{[3-(3,5-Dichloro-phenyl)-propyl]-methanesulfonyl-amino}-heptanoic acid and
5-(3-{[3-(3-Chloro-phenyl)-propyl]-methanesulfonyl-amino}-propyl)-thiophene-2-carboxylic acid.

A group of compounds which is preferred among the R Group of compounds, designated the S Group, contains those compounds wherein
Q is —(C₂-C₆)alkylene-W—(C₁-C₃)alkylene-; and
W is oxy.

A group of compounds which is preferred among the R Group of compounds, designated the T Group, contains those compounds wherein
Q is —(C₃-C₈)alkylene-, said —(C₃-C₈)alkylene- optionally substituted with from one to four fluorines.

Especially preferred compounds among the T Group are compounds wherein
a. A is methylsulfonyl;
   Q is n-hexylene;
   Z is carboxyl;
   K is propylene; and
   M is 3-chlorophenyl; and
b. A is methylsulfonyl;
   Q is n-hexylene;
   Z is carboxyl;
   K is propylene; and
   M is 3,5-dichlorophenyl.

A group of compounds which is preferred among the R Group of compounds, designated the U Group, contains those compounds wherein
Q is —X—(C₁-C₅)alkylene-; and
X is thienyl or phenyl; said phenyl and thienyl optionally mono- or di-substituted independently with fluoro, chloro, trifluoromethyl or methoxy.

A group of compounds which is preferred among the R Group of compounds, designated the V Group, contains those compounds wherein
Q is —(C₁-C₅)alkylene-X—; and
X is thienyl or phenyl; said phenyl and thienyl optionally mono- or di-substituted independently with fluoro, chloro, trifluoromethyl or methoxy.

An especially preferred compound among the V group is a compound wherein
A is methylsulfonyl;
Q-Z is 3-(2-carboxylthien-5-yl)-n-propylene
K is propylene; and
M is 3-chlorophenyl.

A group of compounds which is preferred among the R Group of compounds, designated the W Group, contains those compounds wherein
Q is —(C₁-C₃)alkylene-X—(C₁-C₃)alkylene-; and
X is thienyl or phenyl; said phenyl and thienyl optionally mono- or di-substituted independently with fluoro, chloro, trifluoromethyl or methoxy.

A group of compounds which is preferred among the R Group of compounds, designated the X Group, contains those compounds wherein
Q is —(C₂-C₄)alkylene-W—X—(C₀-C₃)alkylene-;
X is thienyl or phenyl; said phenyl and thienyl optionally mono- or di-substituted independently with fluoro, chloro, trifluoromethyl or methoxy; and
W is oxy.

A group of compounds which is preferred among the R Group of compounds, designated the Y Group, contains those compounds wherein
Q is —(C₀-C₄)alkylene-X—W—(C₁-C₃)alkylene-;
X is thienyl or phenyl; said phenyl and thienyl optionally mono- or di-substituted independently with fluoro, chloro, trifluoromethyl or methoxy; and
W is oxy.

A group of compounds which is preferred among the R Group of compounds, designated the Z Group, contains those compounds wherein
Q is —(C₂-C₄)alkylene-W—X—W—(C₁-C₃)alkylene-;
W is oxy; and
X is thienyl or phenyl; said phenyl and thienyl optionally mono- or di-substituted independently with fluoro, chloro, trifluoromethyl or methoxy.

A group of compounds which is preferred among the R Group of compounds, designated the A1 Group, contains those compounds wherein
Q is —(C₁-C₄)alkylene-ethenylene-(C₁-C₄)alkylene-; and
M is —Ar and —Ar is phenyl, thiazolyl, pyridyl or thienyl.

A group of compounds which is preferred among the R Group of compounds, designated the B1 Group, contains those compounds wherein
Q is —(C₁-C₄)alkylene-ethenylene-(C₀-C₂)alkylene-X—(CO—C₃)alkylene-; and
X is thienyl or phenyl; said phenyl and thienyl optionally mono- or di-substituted independently with fluoro, chloro, trifluoromethyl or methoxy.

A group of compounds which is preferred among the R Group of compounds, designated the C1 Group, contains those compounds wherein
Q is —(C₁-C₃)alkylene-ethenylene-(C₀-C₂)alkylene-X—W—(C₁-C₃)alkylene-;
W is oxy; and
X is thienyl or phenyl; said phenyl and thienyl optionally mono- or di-substituted independently with fluoro, chloro, trifluoromethyl or methoxy.

A group of compounds which is preferred among the R Group of compounds, designated the D1 Group, contains those compounds wherein
Q is —(C₁-C₄)alkylene-ethynylene-(C₁-C₄)alkylene-.

A group of compounds which is preferred among the R Group of compounds, designated the E1 Group, contains those compounds wherein
Q is —(C₁-C₄)alkylene-ethynylene-X—(C₀-C₃)alkylene-; and
X is thienyl or phenyl; said phenyl and thienyl optionally mono- or di-substituted independently with fluoro, chloro, trifluoromethyl or methoxy.

A group of compounds which is preferred among the C Group of compounds, designated the F1 Group, contains those compounds wherein
A is (C₁-C₃)alkylsulfonyl;
K is oxy(C₁-C₄)alkylene;
—Ar is phenyl, thienyl, thiazolyl, pyridyl, benzo[1,3]dioxolyl, cyclopentyl or cyclohexyl; and
R¹, R² and R³ are each independently hydroxy, halo, trifluoromethyl, difluoromethoxy, trifluoromethoxy, (C₁-C₄)alkoxy or (C₁-C₇)alkyl.

Especially preferred compounds within the F1 Group are
7-{[2-(3,5-Dichloro-phenoxy)-ethyl]-methanesulfonyl-amino}-heptanoic acid,
5-(3-{[2-(3,5-Dichloro-phenoxy)-ethyl]-methanesulfonyl-amino}-propyl)-thiophene-2-carboxylic acid and
N-[2-(3,5-Dichloro-phenoxy)-ethyl]-N-[6-(1H-tetrazol-5-yl)-hexyl]-methanesulfonamide.

A group of compounds which is preferred among the F1 Group of compounds, designated the G1 group, contains those compounds wherein
Q is —(C₂-C₆)alkylene-W—(C₁-C₃)alkylene-; and
W is oxy.

A group of compounds which is preferred among the F1 Group of compounds, designated the H1 Group, contains those compounds wherein
Q is —(C₃-C₈)alkylene-, said —(C₃-C₈)alkylene- optionally substituted with from one to four fluorines.

An especially preferred compound among the H1 group of compounds is a compound wherein
A is methylsulfonyl;
Q is n-hexylene;
Z is carboxyl;
K is oxyethylene; and
M is 3,5-dichlorophenyl.

A group of compounds which is preferred among the F1 Group of compounds, designated the I1 Group, contains those compounds wherein
Q is —X—(C₁-C₅)alkylene-; and
X is thienyl or phenyl; said phenyl and thienyl optionally mono- or di-substituted independently with fluoro, chloro, trifluoromethyl or methoxy.

A group of compounds which is preferred among the F1 Group of compounds, designated the J1 Group, contains those compounds wherein
Q is —(C₁-C₅)alkylene-X—; and
X is thienyl or phenyl; said phenyl and thienyl optionally mono- or di-substituted independently with fluoro, chloro, trifluoromethyl or methoxy.

An especially preferred compound among the J1 group is a compound wherein
A is methylsulfonyl;
Q-Z is 3-(2-carboxylthien-5-yl)-n-propylene;
K is oxyethylene; and
M is 3,5-dichlorophenyl.

A group of compounds which is preferred among the F1 Group of compounds, designated the K1 Group, contains those compounds wherein
Q is —(C₁-C₃)alkylene-X—(C₁-C₃)alkylene-; and
X is thienyl or phenyl; said phenyl and thienyl optionally mono- or di-substituted independently with fluoro, chloro, trifluoromethyl or methoxy.

A group of compounds which is preferred among the F1 Group of compounds, designated the L1 Group, contains those compounds wherein
Q is —(C₂-C₄)alkylene-W—X—(C₀-C₃)alkylene-;
X is thienyl or phenyl; said phenyl and thienyl optionally mono- or di-substituted independently with fluoro, chloro, trifluoromethyl or methoxy; and
W is oxy.

A group of compounds which is preferred among the F1 Group of compounds, designated the M1 Group, contains those compounds wherein
Q is —(C₀-C₄)alkylene-X—W—(C₁-C₃)alkylene-;
X is thienyl or phenyl; said phenyl and thienyl optionally mono- or di-substituted independently with fluoro, chloro, trifluoromethyl or methoxy; and W is oxy.

A group of compounds which is preferred among the F1 Group of compounds, designated the N1 Group, contains those compounds wherein
Q is —(C₂-C₄)alkylene-W—X—W—(C₁-C₃)alkylene-;
W is oxy; and
X is thienyl or phenyl; said phenyl and thienyl optionally mono- or di-substituted independently with fluoro, chloro, trifluoromethyl or methoxy.

A group of compounds which is preferred among the F1 Group of compounds, designated the O1 Group, contains those compounds wherein
Q is —(C₁-C₄)alkylene-ethenylene-(C₁-C₄)alkylene-; and
M is —Ar and —Ar is phenyl, thiazolyl, pyridyl or thienyl.

A group of compounds which is preferred among the F1 Group of compounds, designated the P1 Group, contains those compounds wherein
Q is —(C₁-C₄)alkylene-ethenylene-(C₀-C₂)alkylene-X—(CO—C₃)alkylene-; and
X is thienyl or phenyl; said phenyl and thienyl optionally mono- or di-substituted independently with fluoro, chloro, trifluoromethyl or methoxy.

A group of compounds which is preferred among the F1 Group of compounds, designated the Q1 Group, contains those compounds wherein
Q is —(C₁-C₃)alkylene-ethenylene-(C₀-C₂)alkylene-X—W—(C₁-C₃)alkylene-;
W is oxy; and
X is thienyl or phenyl; said phenyl and thienyl optionally mono- or di-substituted independently with fluoro, chloro, trifluoromethyl or methoxy.

A group of compounds which is preferred among the F1 Group of compounds, designated the R1 Group, contains those compounds wherein
Q is —(C₁-C₄)alkylene-ethynylene-(C₁-C₄)alkylene-.

A group of compounds which is preferred among the F1 Group of compounds, designated the S1 Group, contains those compounds wherein
Q is —(C₁-C₄)alkylene-ethynylene-X—(C₀-C₃)alkylene-; and
X is thienyl or phenyl; said phenyl and thienyl optionally mono- or di-substituted independently with fluoro, chloro, trifluoromethyl or methoxy.

A group of compounds which is preferred among the C1 Group of compounds, designated the T1 Group, contains those compounds wherein
A is (C₁-C₃)alkylsulfonyl;
K is (C₃-C₈)alkylene, said (C₃-C₈)alkylene being mono-unsaturated;
—Ar is phenyl, thienyl, thiazolyl, pyridyl, cyclopentyl or cyclohexyl; and

R¹, R² and R³ are each independently hydroxy, halo, trifluoromethyl, difluoromethoxy, trifluoromethoxy, (C₁-C₄)alkoxy or (C₁-C₇)alkyl.

Especially preferred compounds among the T1 Group are
Trans-(4-{[3-(3,5-Dichloro-phenyl)-allyl]-methanesulfonyl-amino}-butoxy)-acetic acid,
Trans-N-[3-(3,5-Dichloro-phenyl)-allyl]-N-[6-(1H-tetrazolyl-5-yl)-hexyl]-methanesulfonamide,
Trans-5-(3-{[3-(3,5-Dichloro-phenyl)-allyl]-methanesulfonyl-amino}-propyl)-thiophene-2-carboxylic acid and
Trans-[3-({[3-(3,5-Dichloro-phenyl)-allyl]-methanesulfonyl-amino}-methyl)-phenyl]-acetic acid.

A group of compounds which is preferred among the T1 Group of compounds, designated the U1 Group, contains those compounds wherein
Q is —(C₂-C₆)alkylene-W—(C₁-C₃)alkylene-; and
W is oxy.
   An especially preferred compound among the U₁ group is a compound wherein
A is methylsulfonyl;
Q is methyloxy-n-butylene;
Z is carboxyl;
K is trans-2-n-propenylene; and

M is 3,5-dichlorophenyl.

A group of compounds which is preferred among the T1 Group of compounds, designated the V1 Group, contains those compounds wherein
Q is —(C₃-C₈)alkylene-, said —(C₃-C₈)alkylene- optionally substituted with from one to four fluorines.

A preferred compound among the V1 group of compound is a compound wherein
A is methylsulfonyl;
Q is n-hexylene;
Z is 5-(1H-tetrazolyl);
K is trans-2-n-propeneylene; and
M is 3,5-dichlorophenyl.

A group of compounds which is preferred among the T1 Group of compounds, designated the W1 Group, contains those compounds wherein
Q is —X—(C₁-C₅)alkylene-; and
X is thienyl or phenyl; said phenyl and thienyl optionally mono- or di-substituted independently with fluoro, chloro, trifluoromethyl or methoxy.

A group of compounds which is preferred among the T1 Group of compounds, designated the X1 Group, contains those compounds wherein
Q is —(C₁-C₅)alkylene-X—; and
X is thienyl or phenyl; said phenyl and thienyl optionally mono- or di-substituted independently with fluoro, chloro, trifluoromethyl or methoxy.

A preferred compound among the X1 Group is a compound wherein
A is methylsulfonyl;
Q-Z is 3-(2-carboxylthien-5-yl)-n-propylene;
K is trans-2-n-propeneylene; and
M is 3,5-dichlorophenyl.

A group of compounds which is preferred among the T1 Group of compounds, designated the Y1 Group, contains those compounds wherein
Q is —(C₁-C₃)alkylene-X—(C₁-C₃)alkylene-; and
X is thienyl or phenyl; said phenyl and thienyl optionally mono- or di-substituted independently with fluoro, chloro, trifluoromethyl or methoxy.

A group of compounds which is preferred among the T1 Group of compounds, designated the Z1 Group, contains those compounds wherein
Q is —(C₂-C₄)alkylene-W—X—(C₀-C₃)alkylene-;
X is thienyl or phenyl; said phenyl and thienyl optionally mono- or di-substituted independently with fluoro, chloro, trifluoromethyl or methoxy; and
W is oxy.

A group of compounds which is preferred among the T1 Group of compounds, designated the A2 Group, contains those compounds wherein
Q is —(C₀-C₄)alkylene-X—W—(C₁-C₃)alkylene-;
X is thienyl or phenyl; said phenyl and thienyl optionally mono- or di-substituted independently with fluoro, chloro, trifluoromethyl or methoxy; and
W is oxy.

A group of compounds which is preferred among the T1 Group of compounds, designated the B2 Group, contains those compounds wherein
Q is —(C₂-C₄)alkylene-W—X—W—(C₁-C₃)alkylene-;
W is oxy; and
X is thienyl or phenyl; said phenyl and thienyl optionally mono- or di-substituted independently with fluoro, chloro, trifluoromethyl or methoxy.

A group of compounds which is preferred among the T1 Group of compounds, designated the C2 Group, contains those compounds wherein
Q is —(C₁-C₄)alkylene-ethenylene-(C₁-C₄)alkylene-; and
M is —Ar and —Ar is phenyl, thiazolyl, pyridyl or thienyl.

A group of compounds which is preferred among the T1 Group of compounds, designated the D2 Group, contains those compounds wherein
Q is —(C₁-C₄)alkylene-ethenylene-(C₀-C₂)alkylene-X—(C₀-C₃)alkylene-; and
X is thienyl or phenyl; said phenyl and thienyl optionally mono- or di-substituted independently with fluoro, chloro, trifluoromethyl or methoxy.

A group of compounds which is preferred among the T1 Group of compounds, designated the E2 Group, contains those compounds wherein
Q is —(C₁-C₃)alkylene-ethenylene-(C₀-C₂)alkylene-X—W—(C₁-C₃)alkylene-;
W is oxy; and
X is thienyl or phenyl; said phenyl and thienyl optionally mono- or di-substituted independently with fluoro, chloro, trifluoromethyl or methoxy.

A group of compounds which is preferred among the T1 Group of compounds, designated the F2 Group, contains those compounds wherein
Q is —(C₁-C₄)alkylene-ethynylene-(C₁-C₄)alkylene-.

A group of compounds which is preferred among the T1 Group of compounds, designated the G2 Group, contains those compounds wherein
Q is —(C₁-C₄)alkylene-ethynylene-X—(C₀-C₃)alkylene-; and
X is thienyl or phenyl; said phenyl and thienyl optionally mono- or di-substituted independently with fluoro, chloro, trifluoromethyl or methoxy.

A preferred group of compounds, designated the H2 Group, contains those compounds having the Formula BB as shown above wherein
B is N;
A is (C₁-C₆)alkanoyl, or (C₃-C₇)cycloalkyl(C₁-C₆)alkanoyl, said A moieties optionally mono-, di- or tri-substituted on carbon independently with hydroxy or halo;
X is phenyl, thienyl, or thiazolyl said phenyl, thienyl or thiazolyl optionally mono- or di-substituted independently with fluoro, chloro, trifluoromethyl, methoxy, difluoromethoxy or trifluoromethoxy;
W is oxy, thio or sulfonyl;
Z is carboxyl, (C₁-C₄)alkoxycarbonyl or tetrazolyl;
K is (C₁-C₈)alkylene or oxy(C₁-C₄)alkylene, said (C₁-C₈)alkylene optionally mono-unsaturated and wherein K is optionally mono-, di- or tri-substituted independently with methyl, fluoro or chloro;
Ar is (C₅-C₇)cycloalkyl, phenyl, thienyl, pyridyl, thiazolyl, oxazolyl, isoxazolyl, naphthalenyl, benzo[b]furanyl, benzo[b]thiophenyl, indanyl, furanyl, benzo[1,3]dioxolyl, benzimidazolyl, benzisoxazolyl, 2,3-dihydrobenzo[1,4]dioxinyl, 2,3-dihydrobenzofuranyl, pyrazolyl, pyrimidyl, pyrazinyl, imidazolyl, quinolinyl, isoquinolinyl, benzoxazolyl, benzothiazolyl, indolyl, 1,2,3,4-tetrahydronaphthalenyl, cyclohexyl, cyclopentyl, or chromanyl;
Ar¹ and Ar² are each independently (C₅-C₇)cycloalkyl, phenyl, thienyl, thiazolyl, pyridyl, pyrimidyl, oxazolyl, furanyl, imidazolyl, isoxazolyl, pyrazinyl or pyrazolyl;
R¹ is halo, (C₁-C₆)alkoxy, (C₁-C₇)alkyl, (C₃-C₇)cycloalkyl, (C₁-C₇)alkanoyl or (C₃-C₇)cycloalkyl(C₁-C₄)alkyl, said (C₁-C₆)alkoxy, (C₁-C₇)alkyl, (C₃-C₇)cycloalkyl, (C₁-C₇)alkanoyl or (C₃-C₇)cycloalkyl(C₁-C₄)alkyl, optionally mono-, di- or tri-substituted independently with hydroxy, fluoro or chloro; and
R² and R³ are each independently hydroxy, halo, difluoromethoxy, trifluoromethoxy, trifluoromethyl, (C₁-C₇)alkyl, (C₁-C₄)alkoxy, (C₁-C₅)alkanoyl, cyano, (C₃-C₇)cycloalkyl, (C₃-C₇)cycloalkyl(C₁-C₄)alkyl, formyl or carbamoyl.

It is especially preferred for the H2 Group that K is not optionally mono-, di- or tri-substituted independently with methyl, fluoro or chloro.

A group of compounds which is preferred among the H2 Group of compounds, designated the 12 Group, contains those compounds wherein
A is (C₁-C₆)alkanoyl, said (C₁-C₆)alkanoyl optionally mono-, di- or tri-substituted on carbon independently with halo;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₂-C₅)alkylene-,
   (C₁-C₅)alkylene-X—,
   (C₁-C₃)alkylene-X—(C₁-C₃)alkylene-,
   (C₂-C₄)alkylene-W—X—(C₀-C₃)alkylene-, or
   (C₀-C₄)alkylene-X—W—(C₁-C₃)alkylene-,
K is methylene or ethylene;
M is —Ar¹—V—Ar² or —Ar¹—O—Ar² wherein Ar¹ and Ar² are each independently phenyl, pyridyl or thienyl;
V is a bond or (C₁-C₂)alkylene;
R¹ is chloro, fluoro, (C₁-C₄)alkyl or (C₁-C₆)alkoxy, said (C₁-C₄)alkyl and (C₁-C₆)alkoxy optionally mono-, di-or tri-substituted independently with hydroxy or fluoro; and
R² and R³ are each independently chloro or fluoro.

A group of compounds which is preferred among the H2 Group of compounds, designated the J2 Group, contains those compounds wherein
A is (C₁-C₆)alkanoyl said (C₁-C₆)alkanoyl optionally mono-, di- or tri-substituted independently on carbon with hydroxy or halo;
K is methylene;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₂-C₅)alkylene-,
   (C₁-C₅)alkylene-X—,
   (C₁-C₃)alkylene-X—(C₁-C₃)alkylene-,
   (C₂-C₄)alkylene-W—X—(C₀-C₃)alkylene-, or
   (CO—C₄)alkylene-X—W—(C₁-C₃)alkylene-;
M is —Ar and —Ar is phenyl, thiazolyl, pyridyl, thienyl, oxazolyl, furanyl, cyclopentyl or cyclohexyl wherein —Ar is substituted with at least R¹;
R¹ is (C₁-C₇)alkyl or (C₁-C₅)alkoxy, said (C₁-C₇)alkyl or (C₁-C₅)alkoxy optionally mono-, di- or tri-substituted independently with hydroxy or fluoro; and
R² and R³ are each independently chloro, fluoro, methyl, difluoromethoxy, trifluoromethoxy or trifluoromethyl.

A group of compounds which is preferred among the H2 Group of compounds, designated the K2 Group, contains those compounds wherein
A is (C₁-C₆)alkanoyl, said (C₁-C₆)alkanoyl optionally mono-, di- or tri-substituted on carbon independently with halo;
K is (C₁-C₈)alkylene;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₂-C₅)alkylene-,
   (C₁-C₅)alkylene-X—,
   (C₁-C₃)alkylene-X—(C₁-C₃)alkylene-,
   (C₂-C₄)alkylene-W—X—(C₀-C₃)alkylene-, or
   (CO—C₄)alkylene-X—W—(C₁-C₃)alkylene-;
M is —Ar and —Ar is phenyl, thienyl, benzofuranyl, benzo[1,3]dioxolyl, 2,3-dihydrobenzo[1,4]dioxinyl, 2,3-dihydrobenzofuranyl, benzimidazolyl, benzo[b]thiophenyl, cyclopentyl or cyclohexyl; and
R¹, R² and R³ are each independently hydroxy, halo, trifluoromethyl, difluoromethoxy, trifluoromethoxy, (C₁-C₄)alkoxy or (C₁-C₇)alkyl.

A group of compounds which is preferred among the H2 Group of compounds, designated the L2 Group, contains those compounds wherein
A is (C₁-C₆)alkanoyl, said (C₁-C₆)alkanoyl optionally mono-, di- or tri-substituted on carbon independently with halo;
K is oxy(C₁-C₄)alkylene;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₂-C₅)alkylene-,
   (C₁-C₅)alkylene-X—,
   (C₁-C₃)alkylene-X—(C₁-C₃)alkylene-,
   (C₂-C₄)alkylene-W—X—(C₀-C₃)alkylene-, or
   (CO—C₄)alkylene-X—W—(C₁-C₃)alkylene-;
M is —Ar and —Ar is phenyl, thienyl, benzo[1,3]dioxolyl, cyclopentyl or cyclohexyl; and
R¹, R² and R³ are each independently hydroxy, halo, trifluoromethyl, difluoromethoxy, trifluoromethoxy, (C₁-C₄)alkoxy or (C₁-C₇)alkyl.

A group of compounds which is preferred among the H2 Group of compounds, designated the M2 Group, contains those compounds wherein
A is (C₃-C₆)alkanoyl said (C₃-C₆)alkanoyl optionally mono-, di- or tri-substituted on carbon independently with halo;
K is (C₃-C₈)alkylene, said (C₃-C₈)alkylene being mono-unsaturated;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₂-C₅)alkylene-,
   (C₁-C₅)alkylene-X—,
   (C₁-C₃)alkylene-X—(C₁-C₃)alkylene-,
   (C₂-C₄)alkylene-W—X—(C₀-C₃)alkylene-, or
   (CO—C₄)alkylene-X—W—(C₁-C₃)alkylene-;
M is —Ar and —Ar is phenyl, thienyl, cyclopentyl or cyclohexyl; and
R¹, R² and R³ are each independently hydroxy, halo, trifluoromethyl, trifluoromethoxy, (C₁-C₄)alkoxy or (C₁-C₇)alkyl.

A preferred group of compounds, designated the N2 Group, contains those compounds having the Formula BB as shown above wherein
B is C(H);
A is (C₁-C₆)alkanoyl, or (C₃-C₇)cycloalkyl(C₁-C₆)alkanoyl, said A moieties optionally mono-, di- or tri-substituted on carbon independently with hydroxy or halo;
X is phenyl, thienyl, or thiazolyl said phenyl, thienyl or thiazolyl optionally mono- or di-substituted independently with fluoro, chloro, trifluoromethyl, methoxy, difluoromethoxy or trifluoromethoxy;
W is oxy, thio or sulfonyl;
Z is carboxyl, (C₁-C₄)alkoxycarbonyl or tetrazolyl;
K is (C₁-C₈)alkylene or oxy(C₁-C₄)alkylene, said (C₁-C₈)alkylene optionally mono-unsaturated and wherein K is optionally mono-, di- or tri-substituted independently with hydroxy, fluoro or chloro;
Ar is (C₅-C₇)cycloalkyl, phenyl, thienyl, pyridyl, thiazolyl, oxazolyl, isoxazolyl, naphthalenyl, benzo[b]furanyl, benzo[b]thiophenyl, indanyl, furanyl, benzo[1,3]dioxolyl, benzimidazolyl, benzisoxazolyl, 2,3-dihydrobenzo[1,4]dioxinyl, 2,3-dihydrobenzofuranyl, pyrazolyl, pyrimidyl, pyrazinyl, imidazolyl, quinolinyl, isoquinolinyl, benzoxazolyl, benzothiazolyl, indolyl, 1,2,3,4-tetrahydronaphthalenyl, cyclohexyl, cyclopentyl, or chromanyl;
Ar¹ and Ar² are each independently (C₅-C₇)cycloalkyl, phenyl, thienyl, thiazolyl, pyridyl, pyrimidyl, oxazolyl, furanyl, imidazolyl, isoxazolyl, pyrazinyl or pyrazolyl;
R¹ is halo, (C₁-C₆)alkoxy, (C₁-C₇)alkyl, (C₃-C₇)cycloalkyl, (C₁-C₇)alkanoyl or (C₃-C₇)cycloalkyl(C₁-C₄)alkyl, said (C₁-C₆)alkoxy, (C₁-C₇)alkyl, (C₃-C₇)cycloalkyl, (C₁-C₇)alkanoyl or (C₃-C₇)cycloalkyl(C₁-C₄)alkyl, optionally mono-, di- or tri-substituted independently with hydroxy, fluoro or chloro; and
R² and R³ are each independently hydroxy, halo, difluoromethoxy, trifluoromethoxy, trifluoromethyl, (C₁-C₇)alkyl, (C₁-C₄)alkoxy, (C₁-C₅)alkanoyl, cyano, (C₃-C₇)cycloalkyl, (C₃-C₇)cycloalkyl(C₁-C₄)alkyl, formyl or carbamoyl.

It is especially preferred for Group N2 that K is not optionally mono-, di- or tri-substituted independently with methyl, fluoro or chloro.

A group of compounds which is preferred among the N2 Group of compounds, designated the O₂ Group, contains those compounds wherein
A is (C₁-C₆)alkanoyl, said A optionally mono-, di- or tri-substituted on carbon independently with halo;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₂-C₅)alkylene-,
   (C₁-C₅)alkylene-X—,
   (C₁-C₃)alkylene-X—(C₁-C₃)alkylene-,
   (C₂-C₄)alkylene-W—X—(C₀-C₃)alkylene-, or
   (C₀-C₄)alkylene-X—W—(C₁-C₃)alkylene-;
K is methylene or ethylene;
M is —Ar¹—V—Ar² or —Ar¹—O—Ar² wherein Ar¹ and Ar² are each independently phenyl, pyridyl or thienyl;
V is a bond or (C₁-C₂)alkylene;
R¹ is chloro, fluoro, (C₁-C₄)alkyl or (C₁-C₄)alkoxy, said (C₁-C₄)alkyl and (C₁-C₄)alkoxy optionally mono-, di-or tri-substituted independently with hydroxy or fluoro; and
R² and R³ are each independently chloro or fluoro.

A group of compounds which is preferred among the N2 Group of compounds, designated the P2 Group, contains those compounds wherein
A is (C₁-C₆)alkanoyl, said A optionally mono-, di- or tri-substituted on carbon independently with hydroxy or halo;
K is methylene;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₂-C₅)alkylene-,
   (C₁-C₅)alkylene-X—,
   (C₁-C₃)alkylene-X—(C₁-C₃)alkylene-,
   (C₂-C₄)alkylene-W—X—(C₀-C₃)alkylene-, or
   (C₀-C₄)alkylene-X—W—(C₁-C₃)alkylene-;
M is —Ar and —Ar is phenyl, thiazolyl, pyridyl, thienyl, oxazolyl, furanyl, cyclopentyl or cyclohexyl wherein —Ar is substituted with at least R¹;
R¹ is (C₁-C₇)alkyl or (C₁-C₆)alkoxy, said (C₁-C₇)alkyl or (C₁-C₆)alkoxy optionally mono-, di- or tri-substituted independently with hydroxy or fluoro; and
R² and R³ are each independently chloro, fluoro, methyl, difluoromethoxy, trifluoromethoxy or trifluoromethyl.

A group of compounds which is preferred among the N2 Group of compounds, designated the Q2 Group, contains those compounds wherein
A is (C₁-C₆)alkanoyl, said A optionally mono-, di- or tri-substituted on carbon independently with halo;
K is (C₁-C₈)alkylene;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₂-C₅)alkylene-,
   (C₁-C₅)alkylene-X—,
   (C₁-C₃)alkylene-X—(C₁-C₃)alkylene-,
   (C₂-C₄)alkylene-W—X—(C₀-C₃)alkylene-, or
   (C₀-C₄)alkylene-X—W—(C₁-C₃)alkylene-;
M is —Ar and —Ar is phenyl, thienyl, benzofuranyl, benzo[1,3]dioxolyl, 2,3-dihydrobenzo[1,4]dioxinyl, 2,3-dihydrobenzofuranyl, benzimidazolyl, benzo[b]thiophenyl, cyclopentyl or cyclohexyl; and
R¹, R² and R³ are each independently hydroxy, halo, trifluoromethyl, trifluoromethoxy, (C₁-C₄)alkoxy or (C₁-C₇)alkyl.

A group of compounds which is preferred among the N2 Group of compounds, designated the R2 Group, contains those compounds wherein
A is (C₁-C₆)alkanoyl said A optionally mono-, di- or tri-substituted on carbon independently with halo;
K is oxy(C₁-C₄)alkylene;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₂-C₅)alkylene-,
   (C₁-C₅)alkylene-X—,
   (C₁-C₃)alkylene-X—(C₁-C₃)alkylene-,
   (C₂-C₄)alkylene-W—X—(C₀-C₃)alkylene-, or
   (C₀-C₄)alkylene-X—W—(C₁-C₃)alkylene-;
M is —Ar and —Ar is phenyl, thienyl, benzo[1,3]dioxolyl, cyclopentyl or cyclohexyl; and
R¹, R² and R³ are each independently hydroxy, halo, trifluoromethyl, trifluoromethoxy, (C₁-C₄)alkoxy or (C₁-C₇)alkyl.

A group of compounds which is preferred among the N2 Group of compounds, designated the S2 Group, contains those compounds wherein
A is (C₁-C₆)alkanoyl, said A optionally mono-, di- or tri-substituted on carbon independently with halo;
K is (C₃-C₈)alkylene, said (C₃-C₈)alkylene being mono-unsaturated;
Q is
   (C₂-C₆)alkylene-W—(C₁-C₃)alkylene-,
   (C₄-C₈)alkylene-, said —(C₄-C₈)alkylene- optionally substituted with up to four substituents independently selected from fluoro or (C₁-C₄)alkyl,
   X—(C₂-C₅)alkylene-,
   (C₁-C₅)alkylene-X—,
   (C₁-C₃)alkylene-X—(C₁-C₃)alkylene-,
   (C₂-C₄)alkylene-W—X—(C₀-C₃)alkylene-, or
   (C₀-C₄)alkylene-X—W—(C₁-C₃)alkylene-;
M is —Ar and —Ar is phenyl, thienyl, cyclopentyl or cyclohexyl; and
R¹, R² and R³ are each independently hydroxy, halo, trifluoromethyl, trifluoromethoxy, (C₁-C₄)alkoxy or (C₁-C₇)alkyl.

An especially preferred compound of the J2 Group of compounds is a compound wherein
A is propanoyl;
Q is n-hexylene;
Z is carboxyl;
K is methylene; and
M is 4-(n-1-hydroxylhexyl)phenyl.

An especially preferred compound among the H1 Group of compounds is a compound wherein
A is methylsulfonyl;
Q is n-hexylene;
Z is 5-(1H-tetrazolyl);
K is oxyethyl; and
M is 3,5-dichlorophenyl.

An especially preferred compound among the Y1 Group of compounds is a compound wherein
A is methylsulfonyl;
Q is 3-methylenephenylmethyl;
Z is carboxyl;
K is trans-2-n-propenylene; and
M is 3,5-dichlorophenyl.

A particularly preferred compound is 7-[(4-butyl-benzyl)-methanesulfonyl-amino]-heptanoic acid or a pharmaceutically acceptable salt or prodrug thereof. A preferred salt is the monosodium salt.

Examples of selective EP₄ compounds that can be used in the present invention include compounds of Formula CC below, which are disclosed in U.S. Pat. No. 6,552,067, issued Apr. 22, 2003. 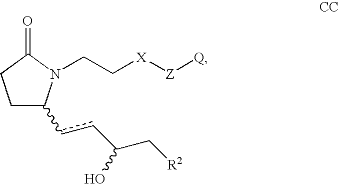
prodrugs thereof, pharmaceutically acceptable salts of said compounds and said prodrugs and stereoisomers and diastereomeric mixtures of said compounds, prodrugs and salts, wherein the dotted line is a bond or no bond; X is —CH₂— or O; Z is —(CH₂)₃—, thienyl, thiazolyl or phenyl, provided that when X is O, then Z is phenyl; Q is carboxyl, (C₁-C₄)alkoxylcarbonyl or tetrazolyl; R² is —Ar or —Ar¹—V—Ar²; V is a bond, —O—, —OCH₂— or —CH₂O—;
Ar is a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, said partially or fully saturated ring or bicyclic ring optionally having one or two oxo groups substituted on carbon or one or two oxo groups substituted on sulfur; and
Ar¹ and Ar² are each independently a partially saturated, fully saturated or fully unsaturated five to eight membered ring optionally having one to four heteroatoms selected independently from oxygen, sulfur and nitrogen, said partially or fully saturated ring optionally having one or two oxo groups substituted on carbon or one or two oxo groups substituted on sulfur;
said Ar moiety is optionally substituted on carbon or nitrogen, on one ring if the moiety is monocyclic, or on one or both rings if the moiety is bicyclic, with up to three substituents per ring each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar are optionally substituted on carbon with up to three fluoro;
said Ar¹ and Ar² moieties are independently optionally substituted on carbon or nitrogen with up to three substituents each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar¹ and Ar² are optionally substituted on carbon with up to three fluoro;
provided that (a) when X is (CH₂)— and Z is —(CH₂)₃—, then R² is not thienyl, phenyl or phenyl monosubstituted with chloro, fluoro, phenyl, methoxy, trifluoromethyl or (C₁-C₄)alkyl; and (b) when X is (CH₂)—, Z is —(CH₂)₃—, and Q is carboxyl or (C₁-C₄)alkoxycarbonyl, then R² is not (i) (C₅-C₇)cycloalkyl or (ii) phenyl, thienyl or furyl each of which may be optionally monosubstituted or disubstituted by one or two substituents selected, independently in the latter case, from halogen atoms, alkyl groups having 1-3 carbon atoms which may be substituted by one or more halogen atoms, and alkoxy groups having 1-4 carbon atoms.

A preferred EP₄ selective compound for use in the present invention is 5-(3-(2S-(3R-hydroxy-4-(3-trifluoromethyl-phenyl)-butyl)-5-oxo-pyrrolidin-1-yl)-propyl)-thiophene-2-carboxylic acid, or a pharmaceutically acceptable salt or prodrug thereof.

Additional examples of EP₄ selective agonists for use in the present invention are disclosed in PCT published patent application WO 01/46140, filed Jun. 28, 2001, which include compounds of Formula DD: 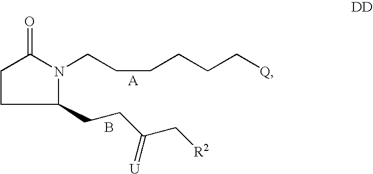
prodrugs thereof and pharmaceutically acceptable salts of said compounds or said prodrugs, wherein:
Q is COOR³, CONHR⁴ or tetrazol-5-yl;
A is a single or cis double bond;
B is a single or trans double bond;
U is 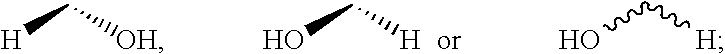
R² is α-thienyl, phenyl, phenoxy, monosubstituted phenyl and monosubstituted phenoxy, said substituents being chloro, fluoro, phenyl, methoxy, trifluoromethyl or (C₁-C₃)alkyl;
R³ is hydrogen, (C₁-C₅)alkyl, phenyl or p-biphenyl;
R⁴ is COR⁵ or SO₂R⁵; and
R⁵ is phenyl or (C₁-C₅)alkyl.

Preferred compounds of the compounds of Formula DD include 7-{2S-[3R-hydroxy-4-(3-phenoxy-phenyl)-butyl]-5-oxo-pyrrolidin-1-yl}-heptanoic acid; 7-(2S-(3R-hydroxy-4-(3-trifluoromethyl-phenyl)-butyl)-5-oxo-pyrrolidin-1-yl)-heptanoic acid; 7-{2S-[4-(3-chloro-phenyl)-3R-hydroxy-butyl]-5-oxo-pyrrolidin-1-yl}-heptanoic acid; 5S-[4-(3-chloro-phenyl)-3R-hydroxy-butyl]-1-[6-(2H-tetrazol-5-yl)-hexyl]-pyrrolidin-2-one; and 5S-(3R-hydroxy-4-(3-trifluoromethyl-phenyl)-butyl)-1-(6-(2H-tetrazol-5-yl)-hexyl)-pyrrolidin-2-one, or the pharmaceutically acceptable salts or prodrugs thereof.

The present invention is also concerned with pharmaceutical compositions for the treatment of metabolic bone disease, senile osteoporosis, postmenopausal osteoporosis, steroid induced osteoporosis, low bone turnover osteoporosis, osteomalacia, renal osteodystrophy, psoriasis, multiple sclerosis, diabetes mellitus, host versus graft rejection, transplant rejection, rheumatoid arthritis, asthma, bone fractures, bone grafts, acne, alopecia, dry skin, insufficient skin firmness, insufficient sebum secretion, wrinkles, hypertension, leukemia, colon cancer, breast cancer, prostate cancer, obesity, osteopenia, male osteoporosis, hypogonadism, andropause, frailty, muscle damage, sarcopenia, osteosarcoma, hypocalcemic tetany, hypoparathyroidism, rickets, vitamin D deficiency, anorexia, low bone mass resulting from aggressive athletic behavior, and for enhancement of peak bone mass in adolescence and prevention of second hip fracture comprising administering to a patient in need thereof a combination of a 2-alkylidene-19-nor-vitamin D derivative, such as a compound of Formula I, and an EP₂ or EP₄ selective agonist or a pharmaceutically acceptable salt or prodrug thereof, and a carrier, solvent, diluent and the like.

It is noted that when compounds are discussed herein, it is contemplated that the compounds may be administered to a patient as a pharmaceutically acceptable salt, prodrug, or a salt of a prodrug. All such variations are intended to be included in the invention.

The term “patient in need thereof” means humans and other animals who have or are at risk of having metabolic bone disease, senile osteoporosis, postmenopausal osteoporosis, steroid induced osteoporosis, low bone turnover osteoporosis, osteomalacia, renal osteodystrophy, psoriasis, multiple sclerosis, diabetes mellitus, host versus graft rejection, transplant rejection, rheumatoid arthritis, asthma, bone fractures, bone grafts, acne, alopecia, dry skin, insufficient skin firmness, insufficient sebum secretion, wrinkles, hypertension, leukemia, colon cancer, breast cancer, prostate cancer, obesity, osteopenia, male osteoporosis, hypogonadism, andropause, frailty, muscle damage, sarcopenia, osteosarcoma, hypocalcemic tetany, hypoparathyroidism, rickets, vitamin D deficiency, anorexia and low bone mass resulting from aggressive athletic behavior and for enhancement of peak bone mass in adolescence and prevention of second hip fracture.

The term “treating”, “treat” or “treatment” as used herein includes preventative (e.g., prophylactic), palliative and curative treatment.

By “pharmaceutically acceptable” it is meant the carrier, diluent, excipients, and/or salts or prodrugs must be compatible with the other ingredients of the formulation, and not deleterious to the patient.

The term “prodrug” means a compound that is transformed in vivo to yield a compound of the present invention. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, when a compound of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, when a compound of the present invention comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

When a compound of the present invention comprises an amine functional group, a prodrug can be formed by the replacement of a hydrogen atom in the amine group with a group such as R^{X}-carbonyl, R^{X}O-carbonyl, NR^{X}R^{X}′-carbonyl where R^{X} and R^{X}′ are each independently (C₁-C₁₀)alkyl, (C₃-C₇)cycloalkyl, benzyl, or R^{X}-carbonyl is a natural α-aminoacyl or natural α-aminoacyl-natural α-aminoacyl, —C(OH)C(O)OY^{X} wherein Y^{X} is H, (C₁-C₆)alkyl or benzyl), —C(OY^{X0})Y^{X1} wherein Y^{X0} is (C₁-C₄)alkyl and Y^{X1} is (C₁-C₆)alkyl, carboxy(C₁-C₆)alkyl, amino(C₁-C₄)alkyl or mono-N- or di-N,N-(C₁-C₆)alkylaminoalkyl, —C(Y^{X2})Y^{X3} wherein Y^{X2} is H or methyl and Y^{X3} is mono-N- or di-N,N-(C₁-C₆)alkylamino, morpholino, piperidin-1-yl or pyrrolidin-1-yl.

The expression “pharmaceutically acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine or tromethamine (2-amino-2-hydroxymethyl-1,3-propanediol).

It will be recognized that the compounds of this invention can exist in radiolabelled form, i.e., said compounds may contain one or more atoms containing an atomic mass or mass number different from the atomic mass or mass number ordinarily found in nature. Radioisotopes of hydrogen, carbon, phosphorous, fluorine and chlorine include ³H, ¹⁴C, ³²P, ³⁵S, ¹⁸F and ³⁶Cl, respectively. Compounds of this invention which contain those radioisotopes and/or other radioisotopes of other atoms are within the scope of this invention. Tritiated, i.e., ³H, and carbon-14, i.e., ¹⁴C, radioisotopes are particularly preferred for their ease of preparation and detectability. Radiolabelled compounds of this invention can generally be prepared by methods well known to those skilled in the art. Conveniently, such radiolabelled compounds can be prepared by carrying out the procedures disclosed herein except substituting a readily available radiolabelled reagent for a non-radiolabelled reagent.

It will be recognized by persons of ordinary skill in the art that some of the compounds of this invention have at least one asymmetric carbon atom and therefore are enantiomers or diastereomers. Diasteromeric mixtures can be separated into their individual diastereomers on the basis of their physicochemical differences by methods known per se as, for example, chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing, including both chemical hydrolysis methods and microbial lipase hydrolysis methods, e.g., enzyme catalyzed hydrolysis) the individual diastereomers to the corresponding pure enantiomers. All such isomers, including diastereomers, enantiomers and mixtures thereof are considered as part of this invention. Also, some of the compounds of this invention are atropisomers (e.g., substituted biaryls) and are considered as part of this invention.

In addition, when the compounds of this invention, including the compounds of Formula I or the EP₂ or EP₄ agonists, form hydrates or solvates, they are also within the scope of the invention.

Administration of the compounds of this invention can be via any method that delivers a compound of this invention systemically and/or locally. These methods include oral, parenteral, and intraduodenal routes, etc. Generally, the compounds of this invention are administered orally, but parenteral administration (e.g., intravenous, intramuscular, transdermal, subcutaneous, rectal or intramedullary) may be utilized, for example, where oral administration is inappropriate for the target or where the patient is unable to ingest the drug.

The compounds of this invention may also be applied locally to a site in or on a patient in a suitable carrier or diluent.

2MD and other 2-alkylidene-19-nor-vitamin D derivatives of the present invention can be administered to a human patient in the range of about 0.01 μg/day to about 10 μg/day. A preferred dosage range is about 0.05 μg/day to about 1 μg/day and a more preferred dosage range is about 0.1 μg/day to about 0.4 μg/day.

In general an effective dosage for the EP₂ or EP₄ agonists used in this invention described above is in the range of 0.001 to 100 mg/kg/day, preferably 0.01 to 50 mg/kg/day.

The amount and timing of administration will, of course, be dependent on the subject being treated, on the severity of the affliction, on the manner of administration and on the judgment of the prescribing physician. Thus, because of patient to patient variability, the dosages given herein are guidelines and the physician may titrate doses of the drug to achieve the treatment that the physician considers appropriate for the patient. In considering the degree of treatment desired, the physician must balance a variety of factors such as age of the patient, presence of preexisting disease, as well as presence of other diseases. The dose may be given once a day or more than once a day and may be given in a sustained release or controlled release formulation. It is also possible to administer the compounds using a combination of an immediate release and a controlled release and/or sustained release formulation.

The administration of 2MD or other 2-alkylidene-19-nor-vitamin D derivative and an EP₂ or EP₄ selective agonist or the combination thereof can be according to any continuous or intermittent dosing schedule. Once a day, multiple times a day, once a week, multiple times a week, once every two weeks, multiple times every two weeks, once a month, multiple times a month, once every two months, once every three months, once every six months and once a year dosing are non-limiting examples of dosing schedules for 2MD or another 2-alkylidene-19-nor-vitamin D derivative and an EP₂ or EP₄ selective agonist or the combination thereof.

The compounds of the present invention are generally administered in the form of a pharmaceutical composition comprising at least one of the compounds of this invention together with a pharmaceutically acceptable vehicle or diluent. Thus, the compounds of this invention can be administered in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof. One example of an acceptable formulation for 2MD and other 2-alkylidene-19-nor-vitamin D derivatives is a soft gelatin capsule containing neobe oil in which the 2MD or other 2-alkylidene-19-nor-vitamin D derivative has been dissolved. Other suitable formulations will be apparent to those skilled in the art.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

The present invention can also be administered using an injectable, flowable composition that provides sustained release at the local site of the injection by forming a biodegradable solid or gel depot, matrix or implant. An example of such an administration system is an EP₂ selective receptor agonist compound in a slow-release biodegradable polymer based delivery system. See, for example, U.S. Published Patent Application No. 2003-0104031 A1.

The polymer based delivery system contains EP₂ selective receptor agonist compound, and optionally any additional therapeutically useful compounds, dissolved or dispersed in biodegradable, thermoplastic polymer solution or dispersion in an organic solvent. Upon injection of the flowable composition, the organic solvent diffuses away from the injection site, causing the polymer to precipitate or gel; thereby entrapping the compound in a sustained-release depot. The compound is subsequently released by diffusion from, and erosion of, the polymeric matrix. The polymeric matrix slowly erodes by hydrolysis and eventually disappears from the site of administration. The molecular weight and concentration of the polymer can control the in vivo release of the compound as well as the degradation rate of the matrix.

The polymer based delivery system provides sustained release of an EP₂ selective receptor agonist compound in vivo for a sustained period of time with minimum or reduced burst in a patient in need thereof. A large burst of compound would result in poor local toleration due to local effects of the compound (e.g., irritation) and would minimize the amount of compound available for efficacy. The advantages this administration method is that it minimizes or reduces the initial burst, but still delivers compound at efficacious levels for a sustained period of time upon a single local injection.

The polymer system is prepared by contacting the flowable composition with a gelation medium to coagulate or gel the composition into a solid, microporous polymeric matrix or a gel polymeric matrix. The flowable composition contains a thermoplastic polymer or copolymer in combination with a suitable solvent. The polymers or copolymers, which form the body of the matrix, are substantially insoluble, preferably essentially completely insoluble, in water and body fluids. The insolubility of the matrix body enables it to function as a single site for the controlled release of the EP₂ selective receptor agonist compound. The polymers or copolymers also are biocompatible and biodegradable and/or bioerodible within the body of an animal, e.g., mammal. The biodegradation enables the patient to metabolize the polymer matrix so that it can be excreted by the patient without the need for further surgery to remove it. Because the flowable composition and polymer system are biocompatible, the insertion process and the presence of the polymer system within the body do not cause substantial tissue irritation or necrosis at the implant site. The composition of the present invention is administered as a flowable composition directly into body tissues.

Suitable thermoplastic polymers for incorporation into the solid matrix of the controlled release polymer system are solids, pharmaceutically compatible and biodegradable by cellular action and/or by the action of body fluids. Examples of appropriate thermoplastic polymers include polyesters of diols and dicarboxylic acids or of hydroxycarboxylic acids, such as polylactides, polyglycolides and copolymers thereof. More preferably the polymer is the copolymer, poly-lactic-co-glycolic acid (abbreviated PLGH), which upon hydrolysis, produces lactic and glycolic acid. The burst of release of this copolymer can be minimized further by the addition of polyethylene glycol (PEG) to form the PEG end-capped PLGH.

Preferred materials for use in the present invention are the polylactides, polyglycolides and copolymers thereof. These polymers can be used to advantage in the polymer system in part because they show excellent biocompatibility. They produce little, if any, tissue irritation, inflammation, necrosis or toxicity. In the presence of water, these polymers produce lactic and glycolic acid, respectively, which are readily metabolized by the body. The polylactides can also incorporate glycolide monomer to enhance the resulting polymer's degradation. These polymers can also be used because they effectively control the rate of release of the EP₂ selective receptor agonist compound from the polymer system, and because they result in the local retention of the EP₂ selective receptor agonist compound at the site of the site of administration.

The solubility or miscibility of a thermoplastic polymer in the organic solvent of the composition will vary according to factors such as crystallinity, hydrophilicity, capacity for hydrogen bonding and molecular weight of the polymer. Consequently, the molecular weight and the concentration of the polymer in the solvent are adjusted to achieve desired miscibility, as well as a desired release rate for the incorporated EP₂ selective receptor agonist compound.

The flowable composition of thermoplastic polymer, solvent and the EP₂ selective receptor agonist compound is a stable flowable substance. A homogenous solution of the EP₂ selective receptor agonist compound in an organic solvent preferably results. The thermoplastic polymer is substantially soluble in the organic solvent. Upon placement of the flowable composition into the body, the solvent will dissipate and the polymer will solidify or gel to form the polymer system having the EP₂ selective receptor agonist compound within a solid or gel polymeric matrix.

It has been discovered that the molecular weight of the polymer used distinctly affects the rate of release of the EP₂ selective receptor agonist compound and the rate of degradation of the polymer from the site as long as the flowable composition has been used as an intermediate.

For certain preferred polymers for use in the present invention, the molecular weight of the polymer or copolymer is adjusted to be within a range of about 0.2 to about 0.4 inherent viscosity (I.V. in deciliters/g) for effective sustained release of the bone growth promoting compound. The typical rate of release of the incorporated bone growth promoting compound occurs at an I.V. of about 0.2 (about 8,000 to about 16,000 molecular weight) or about 0.3 (about 23,000 to about 45,000 molecular weight) but can vary depending on the particular components of the composition. For most systems, it is preferred to adjust the molecular weight of the polymer to about 0.2 I.V. for an effective sustained release of the EP₂ selective receptor agonist compound. The unit of measure for the molecular weight is daltons.

For a poly(DL-lactide) or a lactide-co-glycolide polymer system, the desired molecular weight range is about 0.2 to about 0.4 I.V., with an I.V. of about 0.2 being most preferred. The molecular weight of a polymer can be varied by any of a variety of methods. The choice of method is typically determined by the type of polymer composition. The preferred polymers for use are commercially available.

Highly preferred thermoplastic polymers for use in the present invention are the following: PLGH copolymer with 1:1 ratio of lactic and glycolic acid with an inherent viscosity of about 0.2 dl/g (commercially available from Boehringer Ingelheim as Copolymer RESOMER® RG 502H) (about 12,000 molecular weight); PLGH copolymer with 1:1 ratio of lactic and glycolic acid with an inherent viscosity of about 0.3 dl/g (commercially available from Boehringer Ingelheim as Copolymer RESOMER® RG 503H)(about 37,000 molecular weight); PLGH copolymer with 1:1 ratio of lactic and glycolic acid with an inherent viscosity of about 0.4 dl/g (commercially available from Boehringer Ingelheim as Copolymer RESOMER® RG 504H) (about 47,000 molecular weight); and polyethylene glycol (PEG) end-capped PLGH copolymer with 1:1 ratio of lactic and glycolic acid with an inherent viscosity of about 0.79 dl/g (commercially available from Boehringer Ingelheim as PLG-PEG) (about 52,000 molecular weight).

By appropriate choice of the polymer molecular weight and viscosity, the rate and extent of release of the EP₂ selective receptor agonist compound from the polymer system can be varied from very fast to very slow. For example, according to the present invention, the release rate of (3-(((4-tert-butyl-benzyl)-(pyridine-3-sulfonyl)-amino)-methyl)-phenoxy)-acetic acid, sodium salt, can be slowed to produce substantially complete release of the compound within about seven days. With the use of a greater viscosity of polymer according to the present invention, the period of time can be increased to about fourteen days. The desired release rate of the EP₂ selective receptor agonist compound will depend on several factors, such as the species of animal being treated as well as the specific condition being treated.

The concentration of the polymer in the system can also be varied to adjust the release rate of the incorporated EP₂ selective receptor agonist compound. It has been discovered that the more dilute the polymer concentration, the more readily the EP₂ selective receptor agonist compound compound will be released. This effect can be used in combination with other methods to more effectively control the release of the incorporated EP₂ selective receptor agonist compound as desired. For example, by adjusting the concentration of the polymer and EP₂ selective receptor agonist compound, if desired, a wide range of release rates can be obtained

The solvents used in the thermoplastic compositions of the present invention are preferably pharmaceutically acceptable, biocompatible and will dissipate into body fluid in situ such that they may be classed as having a solubility in water ranging from highly soluble to insoluble. Preferably, they cause relatively little, if any, tissue irritation or necrosis at the site of the injection and implantation. Preferably, the solvent may have at least a minimal degree of water solubility. When the organic solvent is water insoluble or is minimally soluble in water, the solvent will slowly disperse from the flowable polymeric composition. The result will be an implant that during the course of its life may contain a varying amount of residual solvent. Especially preferably, the organic solvent has a moderate to high degree of water solubility so that it will facilely disperse from the polymeric composition into the body fluids. Most preferably, the solvent disperses rapidly from the polymeric composition so as to quickly form a solid implant. Concomitant with the dispersion of solvent, the thermoplastic polymer coagulates or gels into the solid polymer system. Preferably, as the thermoplastic polymer coagulates, the solvent dispersion causes pore formation within the polymer system. As a result, the flowable composition containing thermoplastic polymer, solvent and EP₂ selective receptor agonist compound will form a porous solid polymer system. Also, when the solvent is slightly water soluble or is water insoluble, the solvent dispersion may result in the formation of a solid porous implant, or if some solvent remains with the implant, the result may be formation of a gel implant having few or no pores.

Suitable solvents include those liquid organic compounds meeting the foregoing criteria. The preferred solvent for use in the present invention is N-methyl-2-pyrrolidone (NMP) due, at least in part, to its solvating ability and its biocompatibility.

The solvents for the thermoplastic polymer flowable compositions are chosen for compatibility and appropriate solubility of the polymer and solvent. Lower molecular weight thermoplastic polymers will normally dissolve more readily in the solvents than high molecular weight polymers. As a result, the concentration of a thermoplastic polymer dissolved in the various solvents differs depending upon type of polymer and its molecular weight. Conversely, the higher molecular weight thermoplastic polymers will tend to coagulate, gel or solidify faster than the very low molecular weight thermoplastic polymers. Moreover, the higher molecular weight polymers tend to give higher solution viscosities than the low molecular weight materials. Thus, for advantageous injection efficiency, in addition to advantageous release rate, the molecular weight and the concentration of the polymer in the solvent are controlled.

Upon formation of the polymer system from the flowable composition, the EP₂ selective receptor agonist compound becomes incorporated into the polymer matrix. After insertion of the flowable composition to form in situ the polymer system, the EP₂ selective receptor agonist compound will be released from the matrix into the adjacent tissues or fluids by diffusion and polymer degradation mechanisms. Manipulation of these mechanisms also can influence the release of the EP₂ selective receptor agonist compound into the surroundings at a controlled rate. For example, the polymer matrix can be formulated to degrade after an effective and/or substantial amount of the EP₂ selective receptor agonist compound is released from the matrix. Thus, the release of the EP₂ selective receptor agonist compound from the matrix can be varied by, for example, the solubility of the EP₂ selective receptor agonist compound in water, the distribution of the bone growth promoting compound within the matrix, or the size, shape, porosity, solubility and biodegradability of the polymer matrix, among other factors. The release of the EP₂ selective receptor agonist compound from the matrix is controlled relative to its inherent rate by varying the polymer molecular weight to provide a desired duration and rate of release.

For example, a preferred dosage form of the EP₂ selective receptor agonist compound, (3-(((4-tert-butyl-benzyl)-(pyridine-3-sulfonyl)-amino)-methyl)-phenoxy)-acetic acid, is a lyophile of the sodium salt to be reconstituted with a solution of PLGH in NMP before administration. The dosage form, consisting of the lyophilized compound in one syringe (syringe A) and a solution of PLGH in NMP in a second syringe (syringe B), is known as the A/B reconstitution system. The contents of both syringes are mixed together immediately prior to dose delivery at or near site. After reconstitution, the contents are transferred into a graduated dosing syringe for delivery. The administered dosage forms will be a solution and will result in the dispersion of the compound with PLGH in NMP at desired strengths of, for example, 5 and 50 mgA/ml (mgA/ml refers to the free acid equivalent of the sodium salt form of the compound). The dosage form is a parenteral (e.g., subcutaneous, intramuscular or intramedullary) sustained release injection for local administration. This compound in a slow-release polymer matrix (depot injection) is designed for administration at or near a site, and is not intended for intravenous administration. To provide adequate shelf-life stability for the dosage form, a two-syringe system (A/B), as described above, may be used, preferably with the sodium salt form of the compound. A uniphase formulation, preferably with the free acid form of the compound, is a preferred alternative formulation. Based on the compound and polymer stability, sterile filtration of the compound and irradiation of the polymer solution may be preferred for manufacturing a stable sterile product. In one embodiment, the dosage form can be manufactured and shipped as separate aluminum pouches containing syringes filled with the lyophile form of the compound in one pouch and the polymer solution in the other pouch. Delivery containers, systems and methods for the lyophilization of the bone growth promoting compounds of the present invention to prepare pharmaceutical compositions and kits are described in published International patent application WO 01/73363.

### EXAMPLE A

To obtain dosage form at strengths of 5 and 50 mgA/ml, the following combinations A) and B) of lyophile and polymer syringe, respectively, were used:
A) 5 mgA/ml (upon reconstitution) of (3-(((4-tert-butyl-benzyl)-(pyridine-3-sulfonyl)-amino)-methyl)-phenoxy)-acetic acid, sodium salt formulation:
Drug Syringe A contained 4 mgA of the sodium salt lyophile in 1.25 ml male syringe without graduations; and
Vehicle Syringe B contained 0.8 ml 50% RG502H/50% NMP solution in 1.25 ml female syringe without graduations.
B) 50 mgA/ml (upon reconstitution) of (3-(((4-tert-butyl-benzyl)-(pyridine-3-sulfonyl)-amino)-methyl)-phenoxy)-acetic acid, sodium salt formulation:
Drug Syringe A contained 40 mgA of the sodium salt lyophile in 1.25 ml male (fat) B-D syringe without graduations; and
Vehicle Syringe B contained 0.8 ml 50% RG502H/50% NMP solution in 1.25 ml female (thin) syringe without graduations.
MgA refers to free acid equivalent of the sodium salt form of the compound;
The percentages used in these examples are based on the weight of the indicated ingredients;
RG502H is a PLGH copolymer with 1:1 ratio of lactic and glycolic acid with inherent viscosity of 0.2 dl/gm, which is commercially available such as from Boehringer Ingelheim as Copolymer RESOMER® RG 502H.

### EXAMPLE 1

50% RG502H/50% NMP with 5 mgA/ml of sodium salt of (3-(((4-tert-butyl-benzyl)-(pyridine-3-sulfonyl)-amino)-methyl)-phenoxy)-acetic acid, mixed A/B (polymer solution autoclaved, compound lyophilized).

### EXAMPLE 2

50% RG502H/50% NMP with 10 mgA/ml of sodium salt of (3-(((4-tert-butyl-benzyl)-(pyridine-3-sulfonyl)-amino)-methyl)-phenoxy)-acetic acid, mixed A/B (polymer solution irradiated, compound lyophilized).

### EXAMPLE 3

50% RG502H/50% NMP with 50 mgA/ml of sodium salt of (3-(((4-tert-butyl-benzyl)-(pyridine-3-sulfonyl)-amino)-methyl)-phenoxy)-acetic acid, mixed A/B (polymer solution irradiated, compound lyophilized).

### EXAMPLE 4

47% RG502H/3% PLG-PEG/50% NMP with 50 mgA/ml of sodium salt of (3-(((4-tert-butyl-benzyl)-(pyridine-3-sulfonyl)-amino)-methyl)-phenoxy)-acetic acid, uniphase.

### EXAMPLE 5

47% RG503H/3% PLG-PEG/50% NMP with 50 mgA/ml of sodium salt of (3-(((4-tert-butyl-benzyl)-(pyridine-3-sulfonyl)-amino)-methyl)-phenoxy)-acetic acid, uniphase.

### EXAMPLE 6

45% RG504H/55% NMP with 50 mgA/ml of sodium salt of (3-(((4-tert-butyl-benzyl)-(pyridine-3-sulfonyl)-amino)-methyl)-phenoxy)-acetic acid, uniphase.

### EXAMPLE 7

37% RG503H/63% NMP with 50 mgA/ml of sodium salt of (3-(((4-tert-butyl-benzyl)-(pyridine-3-sulfonyl)-amino)-methyl)-phenoxy)-acetic acid, mixed A/B (polymer solution autoclaved, compound lyophilized).

### EXAMPLE 8

37% RG503H/63% NMP with 50 mgA/ml of sodium salt of (3-(((4-tert-butyl-benzyl)-(pyridine-3-sulfonyl)-amino)-methyl)-phenoxy)-acetic acid, mixed A/B (polymer solution irradiated, compound lyophilized).

### EXAMPLE 9

50% RG502H/50% NMP with 5 mgA/ml of (3-(((4-tert-butyl-benzyl)-(pyridine-3-sulfonyl)-amino)-methyl)-phenoxy)-acetic acid, uniphase.

Further exemplification of the polymer matrix delivery system described above can be found in U.S. provisional patent application No. 60/337,255, filed Nov. 30, 2001.

In a preferred administration system, syringe A contains the lyophile of the sodium salt of (3-(((4-tert-butyl-benzyl)-(pyridine-3-sulfonyl)-amino)-methyl)-phenoxy)-acetic acid which in made to result in 4 mg per syrinige or 40 mg per syringe. Syringe B contains Resomer 502H, 50:50 Poly(D,L lactide-co-glycolide), (50,50 PLGH) and N-methyl-2-pyrroliddone (NMP).

The dose using the above describe A/B syringe system can vary widely and is deterimed by the disese being treated among other factors. A preffered dose range includes 0.5 mgA up to about 100 mgA. In syringes containing 50 mgA/ml, preferred doses include 0.1 ml, 0.2 ml, 0.6 ml and 2 ml. In syringes contianing 5 mgA/ml, preferred doses include 0.1 ml, 0.2 ml, 0,3 ml, 0.6 and 2 ml.

Other methods of administration of an EP₂ selective receptor agonist include local administration by injection to a particular site or delivery by a catheter to a site. Additional examples can be found in U.S. provisional patent application No. 60/335,156, filed Nov. 30, 2001.

Another aspect of the present invention is a kit comprising:
a. an amount of a 2-alkylidene-19-nor-vitamin D derivative, such as a compound of Formula I, and a pharmaceutically acceptable carrier or diluent in a first unit dosage form;
b. an amount of an EP₂ or EP₄ agonist or a pharmaceutically acceptable salt or prodrug thereof, and a pharmaceutically acceptable carrier or diluent in a second unit dosage form; and
c. a container.

The kit comprises two separate pharmaceutical compositions: a 2-alkylidene-19-nor-vitamin D derivative, such as a compound of Formula I and a second compound as described above. The kit comprises container means for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the dosage form so specified should be ingested. Another example of such a memory aid is a calendar printed on the card e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several tablets or capsules to be taken on a given day. Also, a daily dose of a Formula I compound, a prodrug thereof or a pharmaceutically acceptable salt of said compound or said prodrug can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The 2-alkylidene-19-nor-vitamin D derivative and the EP₂ or EP₄ agonist or a pharmaceutically acceptable salt or prodrug thereof can be administered in the same dosage form or in different dosage forms at the same time or at different times. All variations of administration methods are contemplated. A preferred method of administration is to administer the combination in the same dosage form at the same time. Another preferred administration method is to administer the 2-alkylidene-19-nor-vitamin D derivative in one dosage form and the EP₂ or EP₄ agonist in another, both of which are taken at the same time.

The preparation of 1α-hydroxy-2-alkyl-19-nor-vitamin D compounds, particularly 1α-hydroxy-2-methyl-19-nor-vitamin D compounds, having the basic structure I can be accomplished by a common general method, i.e., the condensation of a bicyclic Windaus-Grundmann type ketone II with the allylic phosphine oxide III to the corresponding 2-methylene-19-nor-vitamin D analogs IV followed by deprotection at C-1 and C-3 in the latter compounds:

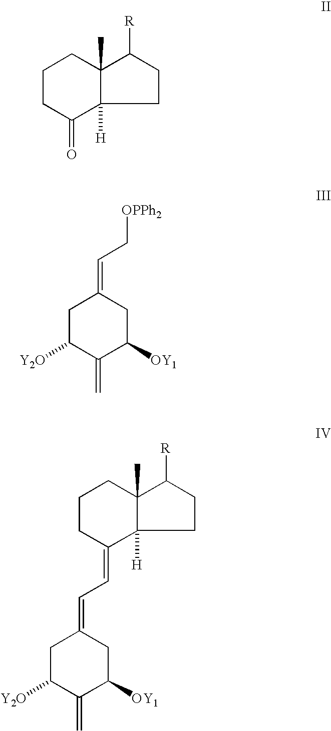

In the structures II, III, and IV groups Y₁ and Y₂ and R represent groups defined above; Y₁ and Y₂ are preferably hydroxy-protecting groups, it being also understood that any functionalities in R that might be sensitive, or that interfere with the condensation reaction, be suitably protected as is well-known in the art. The process shown above represents an application of the convergent synthesis concept, which has been applied effectively for the preparation of vitamin D compounds [e.g., Lythgoe et al., J. Chem. Soc. Perkin Trans. 1, 590 (1978); Lythgoe, Chem. Soc. Rev. 9, 449 (1983); Toh et al., J. Org. Chem. 48, 1414 (1983); Baggiolini et al., J. Org. Chem. 51, 3098 (1986); Sardina et al,. J. Org. Chem. 51, 1264 (1986); J. Org. Chem. 51, 1269 (1986); DeLuca et al., U.S. Pat. No. 5,086,191; DeLuca et al., U.S. Pat. No. 5,536,713].

Hydrindanones of the general structure II are known, or can be prepared by known methods. Specific important examples of such known bicyclic ketones are the structures with the side chains (a), (b), (c) and (d) described above, i.e., 25-hydroxy Grundmann's ketone (f) [Baggiolini et al., J. Org. Chem. 51, 3098 (1986)]; Grundmann's ketone (g) [Inhoffen et al., Chem. Ber. 90, 664 (1957)]; 25-hydroxy Windaus ketone (h) [Baggiolini et al., J. Org. Chem. 51, 3098 (1986)] and Windaus ketone (i) [Windaus et al., Ann., 524, 297 (1936)]:

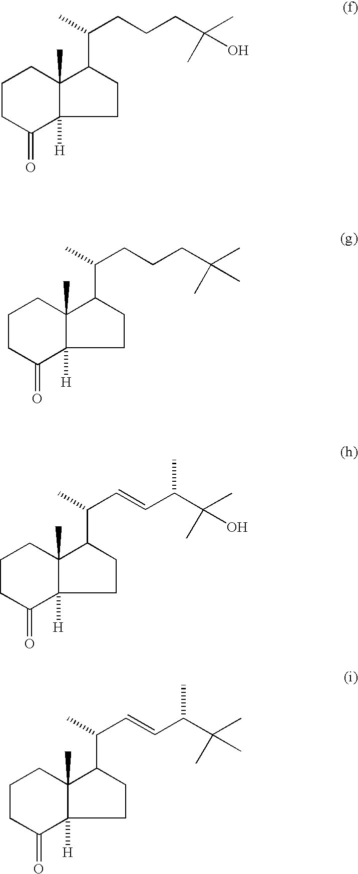

For the preparation of the required phosphine oxides of general structure III, a new synthetic route has been developed starting from methyl quinicate derivative 1, easily obtained from commercial (1R,3R,4S,5R)-(−)-quinic acid as described by Perlman et al., Tetrahedron Lett. 32, 7663 (1991) and DeLuca et al., U.S. Pat. No. 5,086,191. The overall process of transformation of the starting methyl ester 1 into the desired A-ring synthons, is summarized by the Scheme I. Thus, the secondary 4-hydroxyl group of 1 was oxidized with RuO₄ (a catalytic method with RuCl₃ and NalO₄ as co-oxidant). Use of such a strong oxidant was necessary for an effective oxidation process of this very hindered hydroxyl. However, other more commonly used oxidants can also be applied (e.g., pyridinium dichromate), although the reactions usually require much longer time for completion. The second step of the synthesis comprises the Wittig reaction of the sterically hindered 4-keto compound 2 with the ylide prepared from methyltriphenylphosphonium bromide and n-butyllithium. Other bases can be also used for the generation of the reactive methylenephosphorane, like t-BuOK, NaNH₂, NaH, K/HMPT, NaN(TMS)₂, etc. For the preparation of the 4-methylene compound 3 some described modifications of the Wittig process can be used, e.g., reaction of 2 with activated methylenetriphenylphosphorane [Corey et al., Tetrahedron Lett. 26, 555 (1985)]. Alternatively, other methods widely used for methylenation of unreactive ketones can be applied, e.g., Wittig-Horner reaction with the PO-ylid obtained from methyldiphenylphosphine oxide upon deprotonation with n-butyllithium [Schosse et al., Chimia 30, 197 (1976)], or reaction of ketone with sodium methylsulfinate [Corey et al., J. Org. Chem. 28, 1128 (1963)] and potassium methylsulfinate [Greene et al., Tetrahedron Lett. 3755 (1976)]. Reduction of the ester 3 with lithium aluminum hydride or other suitable reducing agent (e.g., DIBALH) provided the diol 4 which was subsequently oxidized by sodium periodate to the cyclohexanone derivative 5. The next step of the process comprises the Peterson reaction of the ketone 5 with methyl(trimethylsilyl)acetate. The resulting allylic ester 6 was treated with diisobutylaluminum hydride and the formed allylic alcohol 7 was in turn transformed to the desired A-ring phosphine oxide 8. Conversion of 7 to 8 involved 3 steps, namely, in situ tosylation with n-butyllithium and p-toluenesulfonyl chloride, followed by reaction with diphenylphosphine lithium salt and oxidation with hydrogen peroxide.

Several 2-methylene-19-nor-vitamin D compounds of the general structure IV may be synthesized using the A-ring synthon 8 and the appropriate Windaus-Grundmann ketone II having the desired side chain structure. Thus, for example, Wittig-Horner coupling of the lithium phosphinoxy carbanion generated from 8 and n-butyllithium with the protected 25-hydroxy Grundmann's ketone 9 prepared according to published procedure [Sicinski et al., J. Med. Chem. 37, 3730 (1994)] gave the expected protected vitamin compound 10. This, after deprotection with AG 50W-X4 cation exchange resin afforded 1α,25-dihydroxy-2-methylene-19-nor-vitamin D₃ (11).

The C-20 epimerization was accomplished by the analogous coupling of the phosphine oxide 8 with protected (20S)-25-hydroxy Grundmann's ketone 13 (SCHEME II) and provided 19-nor-vitamin 14 which after hydrolysis of the hydroxy-protecting groups gave (20S)-1α,25-dihydroxy-2-methylene-19-nor-vitamin D₃ (15). As noted above, other 2-methylene-19-nor-vitamin D analogs may be synthesized by the method disclosed herein. For example, 1α-hydroxy-2-methylene-19-nor-vitamin D₃ can be obtained by providing the Grundmann's ketone (g).

All documents cited in this application, including patents and patent applications, are hereby incorporated by reference. The examples presented below are intended to illustrate particular embodiments of the invention and are not intended to limit the invention, including the claims, in any manner.

### EXAMPLES

The following abbreviations are used in this application.
NMR nuclear magnetic resonance
mp melting point
H hydrogen
h hour(s)
min minutes
t-Bu tert-butyl
THF tetrahydrofuran
n-BuLi n-butyl lithium
MS mass spectra
HPLC high pressure liquid chromatography
SEM standard error measurement
Ph phenyl
Me methyl
Et ethyl
DIBALH diisobutylaluminum hydride
LDA lithium diisopropylamide

The preparation of compounds of Formula I were set forth in U.S. Pat. No. 5,843,928 as follows:

In these examples, specific products identified by Arabic numerals (e.g., 1, 2, 3, etc.) refer to the specific structures so identified in the preceding description and in Scheme I and Scheme II.

### Example 1

### Preparation of 1α,25-dihydroxy-2-methylene-19-nor-vitamin D₃ (11)

Referring first to Scheme I the starting methyl quinicate derivative 1 was obtained from commercial (−)-quinic acid as described previously [Perlman et al., Tetrahedron Lett. 32, 7663 (1991) and DeLuca et al., U.S. Pat. No. 5,086,191]. 1:mp. 82°-82.5° C. (from hexane), ¹H NMR(CDCl₃) δ 0.098, 0.110, 0.142, and 0.159 (each 3H, each s, 4×SiCH₃), 0.896 and 0.911 (9H and 9H, each s, 2×Si-t-Bu), 1.820 (1H, dd, J=13.1, 10.3 Hz), 2.02 (1H, ddd, J=14.3, 4.3, 2.4 Hz), 2.09 (1H, dd, J=14.3, 2.8 Hz), 2.19 (1H, ddd, J=13.1, 4.4, 2.4 Hz), 2.31 (1H, d, J=2.8 Hz, OH), 3.42 (1H, m; after D₂O dd, J=8.6, 2.6 Hz), 3.77 (3H,s), 4.12 (1H,m), 4.37 (1H, m), 4.53 (1H,br s, OH).

(a) Oxidation of 4-hydroxy Group in Methyl Quinicate Derivative 1

(3R,5R)-3,5-Bis[(tert-butyldimethylsilyl)oxy]-1-hydroxy-4-oxocyclohexanecarboxylic Acid Methyl Ester (2). To a stirred mixture of ruthenium (III) chloride hydrate (434 mg, 2.1 mmol) and sodium periodate (10.8 g, 50.6 mmol) in water (42 mL) was added a solution of methyl quinicate 1 (6.09 g, 14 mmol) in CCl₄/CH₃CN (1:1, 64 mL). Vigorous stirring was continued for 8 h. Few drops of 2-propanol were added, the mixture was poured into water and extracted with chloroform. The organic extracts were combined, washed with water, dried (MgSO₄) and evaporated to give a dark oily residue (ca. 5 g) which was purified by flash chromatography. Elution with hexane/ethyl acetate (8:2) gave pure, oily 4-ketone 2 (3.4 g, 56%): ¹H NMR (CDCl₃) δ 0.054, 0.091, 0.127, and 0.132 (each 3H, each s, 4×SiCH₃), 0.908 and 0.913 (9H and 9H, each s, 2×Si-t-Bu), 2.22 (1H, dd, J=13.2, 11.7 Hz), 2.28 (1H, ˜dt J=14.9, 3.6 Hz), 2.37 (1H, dd, J=14.9, 3.2 Hz), 2.55 (1H, ddd, J=13.2, 6.4, 3.4 Hz), 3.79 (3H,s), 4.41 (1H, t, J˜3.5 Hz), 4.64 (1H, s, OH), 5.04 (1H, dd, J=11.7, 6.4 Hz); MS m/z (relative intensity) no M+, 375 (M+-t-Bu, 32), 357 (M+-t-Bu—H₂O, 47), 243 (31), 225 (57), 73 (100).

(b) Wittig Reaction of the 4-ketone 2

(3R,5R)-3,5-Bis[(tert-butyldimethylsilyl)oxy]-1-hydroxy-4-methylenecyclohexanecarboxylic Acid Methyl Ester (3). To the methyltriphenylphoshonium bromide (2.813 g, 7.88 mmol) in anhydrous THF (32 mL) at 0° C. was added dropwise n-BuLi (2.5M in hexanes, 6.0 mL, 15 mmol) under argon with stirring. Another portion of MePh₃P⁺Br⁻ (2.813 g, 7.88 mmol) was then added and the solution was stirred at 0° C. for 10 min. and at room temperature for 40 min. The orange-red mixture was again cooled to 0° C. and a solution of 4-ketone 2 (1.558 g, 3.6 mmol) in anhydrous THF (16+2 mL) was siphoned to reaction flask during 20 min. The reaction mixture was stirred at 0° C. for 1 h. and at room temperature for 3 h. The mixture was then carefully poured into brine cont. 1% HCl and extracted with ethyl acetate and benzene. The combined organic extracts were washed with diluted NaHCO₃ and brine, dried (MgSO₄) and evaporated to give an orange oily residue (ca. 2.6 g) which was purified by flash chromatography. Elution with hexane/ethyl acetate (9:1) gave pure 4-methylene compound 3 as a colorless oil (368 mg, 24%): ¹H NMR (CDCl₃) δ 0.078, 0.083, 0.092, and 0.115 (each 3H, each s, 4×SiCH₃), 0.889 and 0.920 (9H and 9H, each s, 2×Si-t-Bu), 1.811 (1H, dd, J=12.6, 11.2 Hz), 2.10 (2H, m), 2.31 (1H, dd, J=12.6, 5.1 Hz), 3.76 (3H, s), 4.69 (1H, t, J=3.1 Hz), 4.78 (1H, m), 4.96 (2H, m; after D₂O 1H, br s), 5.17 (1H, t, J=1.9 Hz); MS m/z (relative intensity) no M+, 373 (M+-t-Bu, 57), 355 (M+-t-Bu—H₂O, 13), 341 (19), 313 (25), 241 (33), 223 (37), 209 (56), 73 (100).

(c) Reduction of Ester Group in the 4-methylene Compound 3

[(3R,5R)-3,5-Bis[(tert-butyldimethylsilyl)oxy]-1-hydroxy-4-methylenecyclohexyl]methanol (4). (i) To a stirred solution of the ester 3 (90 mg, 0.21 mmol) in anhydrous THF (8 mL) lithium aluminum hydride (60 mg, 1.6 mmol) was added at 0° C. under argon. The cooling bath was removed after 1 h. and the stirring was continued at 6° C. for 12 h. and at room temperature for 6 h. The excess of the reagent was decomposed with saturated aq. Na₂SO₄, and the mixture was extracted with ethyl acetate and ether, dried (MgSO₄) and evaporated. Flash chromatography of the residue with hexane/ethyl acetate (9:1) afforded unreacted substrate (12 mg) and a pure, crystalline diol 4 (35 mg, 48% based on recovered ester 3): ¹H NMR (CDCl₃+D₂O) δ 0.079, 0.091, 0.100, and 0.121 (each 3H, each s, 4×SiCH₃), 0.895 and 0.927 (9H and 9H, each s, 2×Si-t-Bu), 1.339 (1H, t, J˜12 Hz), 1.510 (1H, dd, J=14.3, 2.7 Hz), 2.10 (2H, m), 3.29 and 3.40 (1H and 1H, each d, J=11.0 Hz), 4.66 (1H, t, J˜2.8 Hz), 4.78 (1H, m), 4.92 (1H, t, J=1.7 Hz), 5.13 (1H, t, J=2.0 Hz); MS m/z (relative intensity) no M+, 345 (M+-t-Bu, 8), 327 (M+-t-Bu-H₂O, 22), 213 (28), 195 (11), 73 (100).

(ii) Diisobutylaluminum hydride (1.5M in toluene, 2.0 mL, 3 mmol) was added to a solution of the ester 3 (215 mg, 0.5 mmol) in anhydrous ether (3 mL) at −78° C. under argon. The mixture was stirred at −78° C. for 3 h. and at −24° C. for 1.5 h., diluted with ether (10 mL) and quenched by the slow addition of 2N potassium sodium tartrate. The solution was warmed to room temperature and stirred for 15 min., the poured into brine and extracted with ethyl acetate and ether. The organic extracts were combined, washed with diluted (ca. 1%) HCl, and brine, dried (MgSO₄) and evaporated. The crystalline residue was purified by flash chromatography. Elution with hexane/ethyl acetate (9:1) gave crystalline diol 4 (43 mg, 24%).

(d) Cleavage of the Vicinal Diol 4

(3R,5R)-3,5-Bis[(tert-butyldimethylsilyl)oxy]-4-methylenecyclohexanone (5). Sodium periodate saturated water (2.2 mL) was added to a solution of the diol 4 (146 mg, 0.36 mmol) in methanol (9 mL) at 0° C. The solution was stirred at 0° C. for 1 h., poured into brine and extracted with ether and benzene. The organic extracts were combined, washed with brine, dried (MgSO₄) and evaporated. An oily residue was dissolved in hexane (1 mL) and applied on a silica Sep-Pak cartridge. Pure 4-methylenecyclohexanone derivative 5 (110 mg, 82%) was eluted with hexane/ethyl acetate (95:5) as a colorless oil: ¹H NMR (CDCl₃) δ 0.050 and 0.069 (6H and 6H, each s, 4×SiCH₃), 0.881 (18H, s, 2×Si-t-Bu), 2.45 (2H, ddd, J=14.2, 6.9, 1.4 Hz), 2.64 (2H, ddd, J=14.2, 4.6, 1.4 Hz), 4.69 (2H, dd, J=6.9, 4.6 Hz), 5.16 (2H, s); MS M/z (relative intensity) no M+, 355 (M+-Me, 3), 313 (M+-t-Bu, 100), 73 (76).

(e) Preparation of the Allylic Ester 6

[(3′R,5′R)-3′,5′-Bis[(tert-butyldimethylsilyl)oxy]4′-methylenecyclohexylidene]acetic Acid Methyl Ester (6). To a solution of diisopropylamine (37 μL, 0.28 mmol) in anhydrous THF (200 μL) was added n-BuLi (2.5M in hexanes, 113 μL, 0.28 mmol) under argon at −788 C. with stirring, and methyl(trimethylsilyl)acetate (46 μL, 0.28 mmol) was then added. After 15 min., the keto compound 5 (49 mg, 0.132 mmol) in anhydrous THF (200+80 μL) was added dropwise. The solution was stirred at −78° C. for 2 h. and the reaction mixture was quenched with saturated NH₄Cl, poured into brine and extracted with ether and benzene. The combined organic extracts were washed with brine, dried (MgSO₄) and evaporated. The residue was dissolved in hexane (1 mL) and applied on a silica Sep-Pak cartridge. Elution with hexane and hexane/ethyl acetate (98:2) gave a pure allylic ester 6 (50 mg, 89%) as a colorless oil: ¹H NMR (CDCl₃) δ 0.039, 0.064, and 0.076 (6H, 3H, and 3H, each s, 4×SiCH₃), 0.864 and 0.884 (9H and 9H, each s, 2×Si-t-Bu), 2.26 (1H, dd, J=12.8, 7.4 Hz), 2.47 (1H, dd, J=12.8, 4.2 Hz), 2.98 (1H, dd, J=13.3, 4.0 Hz), 3.06 (1H, dd, J=13.3, 6.6 Hz), 3.69 (3H, s), 4.48 (2H, m), 4.99 (2H, s), 5.74 (1H, s); MS m/z (relative intensity) 426 (M+, 2), 411 (M+-Me, 4), 369 (M+-t-Bu, 100), 263 (69).

(f) Reduction of the Allylic Ester 6

2-[(3′R,5′R)-3′,5′-Bis[(tert-butyldimethylsilyl)oxy]4′-methylenecyclohexylidene]ethanol (7). Diisobutylaluminum hydride (1.5M in toluene, 1.6 mL, 2.4 mmol) was slowly added to a stirred solution of the allylic ester 6 (143 mg, 0.33 mmol) in toluene/methylene chloride (2:1, 5.7 mL) at −78° C. under argon. Stirring was continued as −78° C. for 1 h. and at −46° C. (cyclohexanone/dry ice bath) for 25 min. The mixture was quenched by the slow addition of potassium sodium tartrate (2N, 3 mL), aq. HCl (2N, 3 mL) and H₂O (12 mL), and then diluted with methylene chloride (12 mL) and extracted with ether and benzene. The organic extracts were combined, washed with diluted (ca. 1%) HCl, and brine, dried (MgSO₄) and evaporated. The residue was purified by flash chromatography. Elution with hexane/ethyl acetate (9:1) gave crystalline allylic alcohol 7 (130 mg, 97%): ¹H NMR (CDCl₃) δ 0.038, 0.050, and 0.075 (3H, 3H, and 6H, each s, 4×SiCH₃), 0.876 and 0.904 (9H and 9H, each s, 2×Si-t-Bu), 2.12 (1H, dd J=12.3, 8.8 Hz), 2.23 (1H, dd, J=13.3, 2.7 Hz), 2.45 (1H, dd, J=12.3, 4.8 Hz), 2.51 (1H, dd, J=13.3, 5.4 Hz), 4.04 (1H, m; after D₂O dd, J=12.0, 7.0 Hz), 4.17 (1H, m; after D₂O dd, J=12.0, 7.4 Hz), 4.38 (1H, m), 4.49 (1H, m), 4.95 (1H, br s), 5.05 (1H, t, J=1.7 Hz), 5.69 (1H, ˜t, J=7.2 Hz); MS m/z (relative intensity) 398 (M+, 2), 383 (M+-Me, 2), 365 (M+-Me-H₂O, 4), 341 (M+-t-Bu, 78), 323 (M+-t-Bu-H₂O, 10), 73 (100).

(g) Conversion of the Allylic Alcohol 7 into Phosphine Oxide 8

[2-[(3′R,5′R)-3′,5′-Bis[(tert-butyldimethylsilyl)oxy]-4′-methylenecyclohexylidene]ethyl]diphenylphosphine Oxide (8). To the allylic alcohol 7 (105 mg, 0.263 mmol) in anhydrous THF (2.4 mL) was added n-BuLi (2.5M in hexanes, 105 μL, 0.263 mmol) under argon at 0° C. Freshly recrystallized tosyl chloride (50.4 mg, 0.264 mmol) was dissolved in anhydrous THF (480 μL) and added to the allylic alcohol-BuLi solution. The mixture was stirred at 0° C. for 5 min. and set aside at 0° C. In another dry flask with air replaced by argon, n-BuLi (2.5M in hexanes, 210 μL, 0.525 mmol) was added to Ph₂PH (93 μL, 0.534 mmol in anhydrous THF (750 μL) at 0° C. with stirring. The red solution was siphoned under argon pressure to the solution of tosylate until the orange color persisted (ca. ½ of the solution was added). The resulting mixture was stirred an additional 30 min. at 0° C., and quenched by addition of H₂O (30 μL). Solvents were evaporated under reduced pressure and the residue was redissolved in methylene chloride (2.4 mL) and stirred with 10% H₂O₂ at 0° C. for 1 h. The organic layer was separated, washed with cold aq. Sodium sulfite and H₂O, dried (MgSO₄) and evaporated. The residue was subject to flash chromatography. Elution with benzene/ethyl acetate (6:4) gave semicrystalline phosphine oxide 8 (134 mg, 87%): ¹H NMR (CDCl₃) δ 0.002, 0.011 and 0.019 (3H, 3H, and 6H, each s, 4×SiCH₃), 0.855 and 0.860 (9H and 9H, each s, 2×Si-t-Bu), 2.0-2.1 (3H, br m), 2.34 (1H, m), 3.08 (1H, m), 3.19 (1H, m), 4.34 (2H, m), 4.90 and 4.94 (1H and 1H, each s,), 5.35 (1H, ˜q, J=7.4 Hz), 7.46 (4H, m), 7.52 (2H, m), 7.72 (4H, m); MS m/z (relative intensity) no M+, 581 (M+−1, 1), 567 (M+-Me, 3) 525 (M+-t-Bu, 100), 450 (10), 393 (48).

(h) Wittig-Horner Coupling of Protected 25-hydroxy Grundmann's Ketone 9 with the Phosphine Oxide 8

1α,25-Dihydroxy-2-methylene-19-nor-vitamin D₃ (11). To a solution of phosphine oxide 8 (33.1 mg, 56.8 μmol) in anhydrous THF (450 μL) at 0° C. was slowly added n-BuLi (2.5M in hexanes, 23 μL, 57.5 μmol) under argon with stirring. The solution turned deep orange. The mixture was cooled to −78° C. and a precooled (−78° C.) solution of protected hydroxy ketone 9 (9.0 mg, 22.8 μmol), prepared according to published procedure [Sicinski et al., J. Med. Chem. 37, 3730 (1994)], in anhydrous THF (200+100 μL) was slowly added. The mixture was stirred under argon at −78° C. for 1 h. and at 0° C. for 18 h. Ethyl acetate was added, and the organic phase was washed with brine, dried (MgSO₄) and evaporated. The residue was dissolved in hexane and applied on a silica Sep-Pak cartridge, and washed with hexane/ethyl acetate (99:1, 20 mL) to give 19-nor-vitamin derivative 10 (13.5 mg, 78%). The Sep-Pak was then washed with hexane/ethyl acetate (96:4), 10 mL) to recover some unchanged C,D-ring ketone 9 (2 mg), and with ethyl acetate (10 mL) to recover diphenylphosphine oxide (20 mg). For analytical purpose a sample of protected vitamin 10 was further purified by HPLC (6.2 mm×25 cm Zorbax-Sil column, 4 mL/min) using hexane/ethyl acetate (99.9:0.1) solvent system. Pure compound 10 was eluted at Rᵥ 26 mL as a colorless oil: UV (in hexane) λₘₐₓ 224, 253, 263 nm; ¹H NMR (CDCl₃) δ 0.025, 0.049, 0.066, and 0.080 (each 3H, each s, 4×SiCH₃), 0.546 (3H, s, 18-H₃), 0.565 (6H, q, J=7.9 Hz, 3×SiCH₂), 0.864 and 0.896 (9H and 9H, each s, 2×Si-t-Bu), 0.931 (3H, d, J=6.0 Hz, 21-H₃), 0.947 (9H, t, J=7.9 Hz, 3×SiCH₂CH₃), 1.188 (6H, s, 26- and 27-H₃), 2.00 (2H, m), 2.18 (1H, dd, J=12.5, 8.5 Hz, 4β-H), 2.33 (1H, dd, J=13.1, 2.9 Hz, 10α-H), 2.46 (1H, dd J=12.5, 4.5 Hz, 4α-H), 2.52 (1H, dd, J=13.1, 5.8 Hz, 10α-H), 2.82 (1H, brd, J=12 Hz, 9β-H), 4.43 (2H, m, 1β- and 3α-H), 4.92 and 4.97 (1H and 1H, each s, ═CH₂), 5.84 and 6.22 (1H and 1H, each d, J=11.0 Hz, 7- and 6-H); MS m/z (relative intensity) 758 (M+, 17), 729 (M+-Et, 6), 701 (M+-t-Bu, 4), 626 (100), 494 (23), 366 (50), 73 (92).

Protected vitamin 10 (4.3 mg) was dissolved in benzene (150 μL) and the resin (AG 50W-X4, 60 mg; prewashed with methanol) in methanol (800 μL) was added. The mixture was stirred at room temperature under argon for 17 h., diluted with ethyl acetate/ether (1:1, 4 mL) and decanted. The resin was washed with ether (8 mL) and the combined organic phases washed with brine and saturated NaHCO₃, dried (MgSO₄) and evaporated. The residue was purified by HPLC (62 mm×25 cm Zorbax-Sil column, 4 mL/min.) using hexane/2-propanol (9:1) solvent system. Analytically pure 2-methylene-19-nor-vitamin 11 (2.3 mg, 97%) was collected at Rᵥ 29 mL (1α,25-dihydroxyvitamin D₃ was eluted at Rᵥ 52 mL in the same system) as a white solid: UV (in EtOH) λₘₐₓ 243.5, 252, 262.5 nm; ¹H NMR (CDCl₃) δ 0.552 (3H, s, 18-H₃), 0.941 (3H, d, J=6.4 Hz, 21-H₃), 1.222 (6H, s, 26- and 27-H₃), 2.01 (2H, m), 2.27-2.36 (2H, m), 2.58 (1H, m), 2.80-2.88 (2H, m), 4.49 (2H, m, 1β- and 3α-H), 5.10 and 5.11 (1H and 1H, each s, ═CH₂), 5.89 and 6.37 (1H and 1H, each d, J=11.3 Hz, 7- and 6-H); MS m/z (relative intensity) 416 (M+, 83), 398 (25), 384 (31), 380 (14), 351 (20), 313 (100).

### Example 2

Preparation of (20S)-1α,25-dihydroxy-2-methylene-19-nor-vitamin D₃ (15)

Scheme II illustrates the preparation of protected (20S)-25-hydroxy Grundmann's ketone 13, and its coupling with phosphine oxide 8 (obtained as described in Example 1).

(a) Silylation of Hydroxy Ketone 12

(20S)-25-[(Triethylsilyl)oxy]-des-A,B-cholestan-8-one (13). A solution of the ketone 12 (Tetrionics, Inc. Madison, Wis.; 56 mg, 0.2 mmol) and imidazole (65 mg, 0.95 mmol) in anhydrous DMF (1.2 mL) was treated with triethylsilyl chloride (95 μL, 0.56 mmol), and the mixture was stirred at room temperature under argon for 4 h. Ethyl acetate was added and water, and the organic layer was separated. The ethyl acetate layer was washed with water and brine, dried (MgSO₄) and evaporated. The residue was passed through a silica Sep-Pak cartridge in hexane/ethyl acetate (9:1) and after evaporation, purified by HPLC (9.4 mm×25 cm Zorbax-Sil column, 4 mL/min) using hexane/ethyl acetate (9:1) solvent system. Pure protected hydroxy ketone 13 (55 mg, 70%) was eluted at Rᵥ 35 mL as a colorless oil: ¹H NMR (CDCl₃) δ 0.566 (6H, q, J=7.9 Hz, 3×SiCH₂), 0.638 (3H, s, 18-H₃), 0.859 (3H, d, J=6.0 Hz, 21-H₃), 0.947 (9H, t, J=7.9 Hz, 3×SiCH₂CH₃), 1.196 (6H, s, 26- and 27-H₃), 2.45 (1H, dd, J=11.4, 7.5 Hz, 14α-H).

(b) Wittig-Horner Coupling of Protected (20S)-25-hydroxy Grundmann's Ketone 13 with the Phosphine Oxide 8

(20S)-1α,25-Dihydroxy-2-methylene-19-nor-vitamine D₃ (15). To a solution of phosphine oxide 8 (15.8 mg, 27.1 μmol) in anhydrous THF (200 μL) at 0° C. was slowly added n-BuLi (2.5M in hexanes, 11 μL, 27.5 μmol) under argon with stirring. The solution turned deep orange. The mixture was cooled to −78° C. and a precooled (−78° C.) solution of protected hydroxy ketone 13 (8.0 mg, 20.3 μmol) in anhydrous THF (100 μL) was slowly added. The mixture was stirred under argon at −78° C. for 1 h. and at 0° C. for 18 h. Ethyl acetate was added, and the organic phase was washed with brine, dried (MgSO₄) and evaporated. The residue was dissolved in hexane and applied on a silica Sep-Pak cartridge, and washed with hexane/ethyl acetate (99.5:0.5, 20 mL) to give 19-nor-vitamin derivative 14 (7 mg, 45%) as a colorless oil. The Sep-Pak was then washed with hexane/ethyl acetate (96:4, 10 mL) to recover some unchanged C,D-ring ketone 13 (4 mg), and with ethyl acetate (10 mL) to recover diphenylphosphine oxide (9 mg). For analytical purpose a sample of protected vitamin 14 was further purified by HPLC (6.2 mm×25 cm Zorbax-Sil column, 4 mL/min) using hexane/ethyl acetate (99.9:0.1) solvent system.

14: UV (in hexane) λₘₐₓ 244, 253.5, 263 nm; ¹H NMR (CDCl₃) δ 0.026, 0.049, 0.066 and 0.080 (each 3H, each s, 4×SiCH₃), 0.541 (3H, s, 18-H₃), 0.564 (6H, q, J=7.9 Hz, 3×SiCH₂), 0.848 (3H, d, J=6.5 Hz, 21-H₃), 0.864 and 0.896 (9H and 9H, each s, 2×Si-t-Bu), 0.945 (9H, t, J=7.9 Hz, 3×SiCH₂CH₃), 1.188 (6H, s, 26- and 27-H₃), 2.15-2.35 (4H, br m), 2.43-2.53 (3H, br m), 2.82 (1H, br d, J=12.9 Hz, 9β-H), 4.42 (2H, m, 1β- and 3α-H), 4.92 and 4.97 (1H and 1H, each s, ═CH₂), 5.84 and 6.22 (1H and 1H, each d, J=11.1 Hz, 7- and 6-H); MS m/z (relative intensity) 758 (M+, 33), 729 (M+-Et, 7), 701 (M+-t-Bu, 5), 626 (100), 494 (25), 366 (52), 75 (82), 73 (69).

Protected vitamin 14 (5.0 mg) was dissolved in benzene (160 μL) and the resin (AG 50W-X4, 70 mg; prewashed with methanol) in methanol (900 μL) was added. The mixture was stirred at room temperature under argon for 19 h. diluted with ethyl acetate/ether (1:1, 4 mL) and decanted. The resin was washed with ether (8 mL) and the combined organic phases washed with brine and saturated NaHCO₃, dried (MgSO₄) and evaporated. The residue was purified by HPLC (6.2 mm×25 cm Zorbax-Sil column, 4 mL/min.) using hexane/2-propanol (9:1) solvent system. Analytically pure 2-methylene-19-nor-vitamin 15 (2.6 mg, 95%) was collected at Rᵥ 28 mL [(20R)-analog was eluted at Rᵥ 29 mL and 1α,25-dihydroxyvitamin D₃ at Rᵥ 52 mL in the same system] as a white solid: UV (in EtOH) λₘₐₓ 243.5, 252.5, 262.5 nm; ³H NMR (CDCl₃) δ 0.551 (3H, s, 18-H₃), 0.858 (3H, d, J=6.6 Hz, 21-H₃), 1.215 (6H, s, 26- and 27-H₃), 1.95-2.04 (2H, m), 2.27-2.35 (2H, m), 2.58 (1H, dd, J=13.3, 3.0 Hz), 2.80-2.87 (2H, m), (2H, m, 1β- and 3α-H), 5.09 and 5.11 (1H and 1H, each s, ═CH₂), 5.89 and 6.36 (1H and 1H, each d, J=11.3 Hz, 7- and 6-H); MS m/z (relative intensity) 416 (M+, 100), 398 (26), 380 (13), 366 (21), 313 (31).

Biological Activity of 2-Methylene-Substituted 19-NOR-1,25-(OH)₂D₃ Compounds and their 20S-Isomers

The biological activity of compounds of Formula I was set forth in U.S. Pat. No. 5,843,928 as follows. The introduction of a methylene group to the 2-position of 19-nor-1,25-(OH)₂D₃ or its 20S-isomer had little or no effect on binding to the porcine intestinal vitamin D receptor. All compounds bound equally well to the porcine receptor including the standard 1,25-(OH)₂D₃. It might be expected from these results that all of the compounds would have equivalent biological activity. Surprisingly, however, the 2-methylene substitutions produced highly selective analogs with their primary action on bone. When given for 7 days in a chronic mode, the most potent compound tested was the 2-methylene-19-nor-20S-1,25-(OH)₂D₃ (Table 1). When given at 130 pmol/day, its activity on bone calcium mobilization (serum calcium) was of the order of at least 10 and possible 100-1,000 times more than that of the native hormone. Under identical conditions, twice the dose of 1,25-(OH)₂D₃ gave a serum calcium value of 13.8 mg/100 ml of serum calcium at the 130 pmol dose. When given at 260 pmol/day, it produced the astounding value of 14 mg/100 ml of serum calcium at the expense of bone. To show its selectivity, this compound produced no significant change in intestinal calcium transport at either the 130 or 260 pmol dose, while 1,25-(OH)₂D₃ produced the expected elevation of intestinal calcium transport at the only dose tested, i.e. 260 pmol/day. The 2-methylene-19-nor-1,25-(OH)₂D₃ also had extremely strong bone calcium mobilization at both dose levels but also showed no intestinal calcium transport activity. The bone calcium mobilization activity of this compound is likely to be 10-100 times that of 1,25-(OH)₂D₃. These results illustrate that the 2-methylene and the 20S-2-methylene derivatives of 19-nor-1,25-(OH)₂D₃ are selective for the mobilization of calcium from bone. Table 2 illustrates the response of both intestine and serum calcium to a single large dose of the various compounds; again, supporting the conclusions derived from Table 1.

The results illustrate that 2-methylene-19-nor-20S1,25-(OH)₂D₃ is extremely potent in inducing differentiation of HL-60 cells to the monocyte. The 2-methylene-19-nor compound had activity similar to 1,25-(OH)₂D₃. These results illustrate the potential of the 2-methylene-19-nor-20S-1,25-(OH)₂D₃ and 2-methylene-19-nor-1,25-(OH)₂D₃ compounds as anti-cancer agents, especially against leukemia, colon cancer, breast cancer and prostate cancer, or as agents in the treatment of psoriasis.

Competitive binding of the analogs to the porcine intestinal receptor was carried out by the method described by Dame et al. (Biochemistry 25, 4523-4534, 1986).

The differentiation of HL-60 promyelocytic into monocytes was determined as described by Ostrem et al (J. Biol. Chem. 262, 14164-14171, 1987).

TABLE 1
Response of Intestinal Calcium Transport and Serum
Calcium (Bone Calcium Mobilization) Activity to Chronic
Doses of 2-Methylene Derivatives of 19-Nor-1,25-
(OH)₂D₃ and its 20S Isomers

| | Dose | Intestinal Calcium | Serum |
|---|---|---|---|
| | (pmol/day/ | Transport | Calcium |
| Group | 7 days) | (S/M) | (mg/100 ml) |
| Vitamin D Deficient | Vehicle | 5.5 ± 0.2 | 5.1 ± 0.16 |
| 1,25-(OH)₂D₃ Treated | 260 | 6.2 ± 0.4 | 7.2 ± 0.5 |
| 2-Methylene-19-Nor-1,25- | 130 | 5.3 ± 0.4 | 9.9 ± 0.2 |
| (OH)₂D₃ | 260 | 4.9 ± 0.6 | 9.6 ± 0.3 |
| 2-Methylene-19-Nor-20S- | 130 | 5.7 ± 0.8 | 13.8 ± 0.5 |
| 1,25-(OH)₂D₃ | 260 | 4.6 ± 0.7 | 14.4 ± 0.6 |

Male weanling rats were obtained from Sprague Dawley Co. (Indianapolis, Ind.) and fed a 0.47% calcium, 0.3% phosphorus vitamin D-deficient diet for 1 week and then given the same diet containing 0.02% calcium, 0.3% phosphorus for 2 weeks. During the last week they were given the indicated dose of compound by intraperitoneal injection in 0.1 ml 95% propylene glycol and 5% ethanol each day for 7 days. The control animals received only the 0.1 ml of 95% propylene glycol, 5% ethanol. Twenty-four hours after the last dose, the rats were sacrificed and intestinal calcium transport was determined by everted sac technique as previously described and serum calcium determined by atomic absorption spectrometry on a model 3110 Perkin Elmer instrument (Norwalk, Conn.). There were 5 rats per group and the values represent mean (±)SEM.

TABLE 2
Response of Intestinal Calcium Transport and Serum
Calcium (Bone Calcium Mobilization) Activity to Chronic
Doses of 2-Methylene Derivatives of 19-Nor-1,25-
(OH)₂D₃ and its 20S Isomers

| | Intestinal Calcium | |
|---|---|---|
| | Transport | Serum Calcium |
| Group | (S/M) | (mg/100 ml) |
| -D Control | 4.2 ± 0.3 | 4.7 ± 0.1 |
| 1,25-(OH)₂D₃ | 5.8 ± 0.3 | 5.7 ± 0.2 |
| 2-Methylene-19-Nor-1,25-(OH)₂D₃ | 5.3 ± 0.5 | 6.4 ± 0.1 |
| 2-Methylene-19-Nor-20S-1,25- | 5.5 ± 0.6 | 8.0 ± 0.1 |
| (OH)₂D₃ | | |

Male Holtzman strain weanling rats were obtained from the Sprague Dawley Co. (Indianapolis, Ind.) and fed the 0.47% calcium, 0.3% phosphorus diet described by Suda et al. (J. Nutr. 100, 1049-1052, 1970) for 1 week and then fed the same diet containing 0.02% calcium and 0.3% phosphorus for 2 additional weeks. At this point, they received a single intrajugular injection of the indicated dose dissolved in 0.1 ml of 95% propylene glycol/5% ethanol. Twenty-four hours later they were sacrificed and intestinal calcium transport and serum calcium were determined as described in Table 1. The dose of the compounds was 650 pmol and there were 5 animals per group. The data are expressed as mean (±)SEM.

Accordingly, compounds of the following formulae Ia, are along with those of formula I, also encompassed by the present invention:

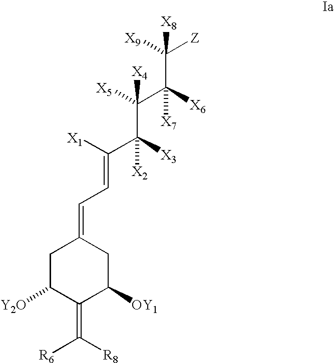

In the above formula Ia, the definitions of Y₁, Y₂, R₆, R₈ and Z are as previously set forth herein. With respect to X₁, X₂, X₃, X₄, X₅, X₆, X₇, X₈ and X₉, these substituents may be the same or different and are selected from hydrogen or lower alkyl, i.e., a C₁₋₅ alkyl such as a methyl, ethyl or n-propyl. In addition, paired substituents X₁ and X₄, or X₅, X₂ or X₃ and X₆ or X₇, X₄ or X₅ and X₈ or X₉, when taken together with the three adjacent carbon atoms of the central part of the compound, which correspond to positions 8, 14, 13 or 14, 13, 17 or 13, 17, 20 respectively, can be the same or different and form a saturated or unsaturated, substituted or unsubstituted, carbocyclic 3, 4, 5, 6 or 7 membered ring.

Preferred compounds of the present invention may be represented by one of the following formulae:

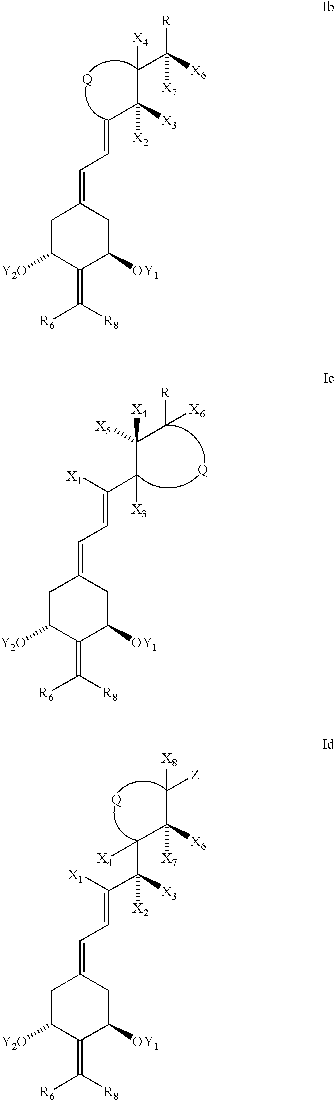

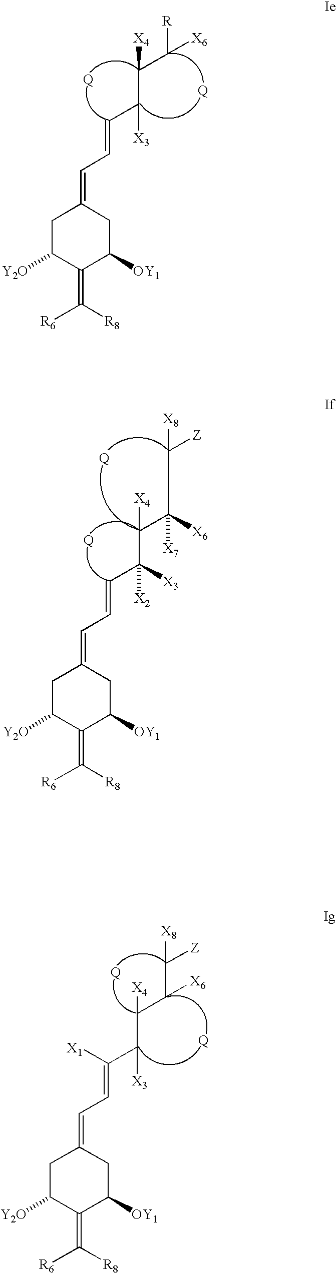

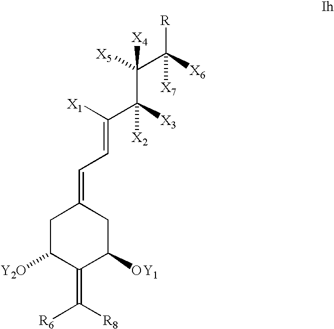

In the above formulae Ib, Ic, Id, le, If, Ig and 1 h, the definitions of Y₁, Y₂, R₆, R₈, R, Z, X₁, X₂, X₃, X₄, X₅, X₆, X₇, and X₈ are as previously set forth herein. The substituent Q represents a saturated or unsaturated, substituted or unsubstituted, hydrocarbon chain comprised of 0, 1, 2, 3 or 4 carbon atoms, but is preferably the group —(CH₂)ₖ— where k is an integer equal to 2 or 3.

Methods for making compounds of formulae Ia-Ih are known. Specifically, reference is made to International Application Number PCT/EP94/02294 filed Jul. 7, 1994, and published Jan. 19, 1995, under International Publication Number WO95/01960.

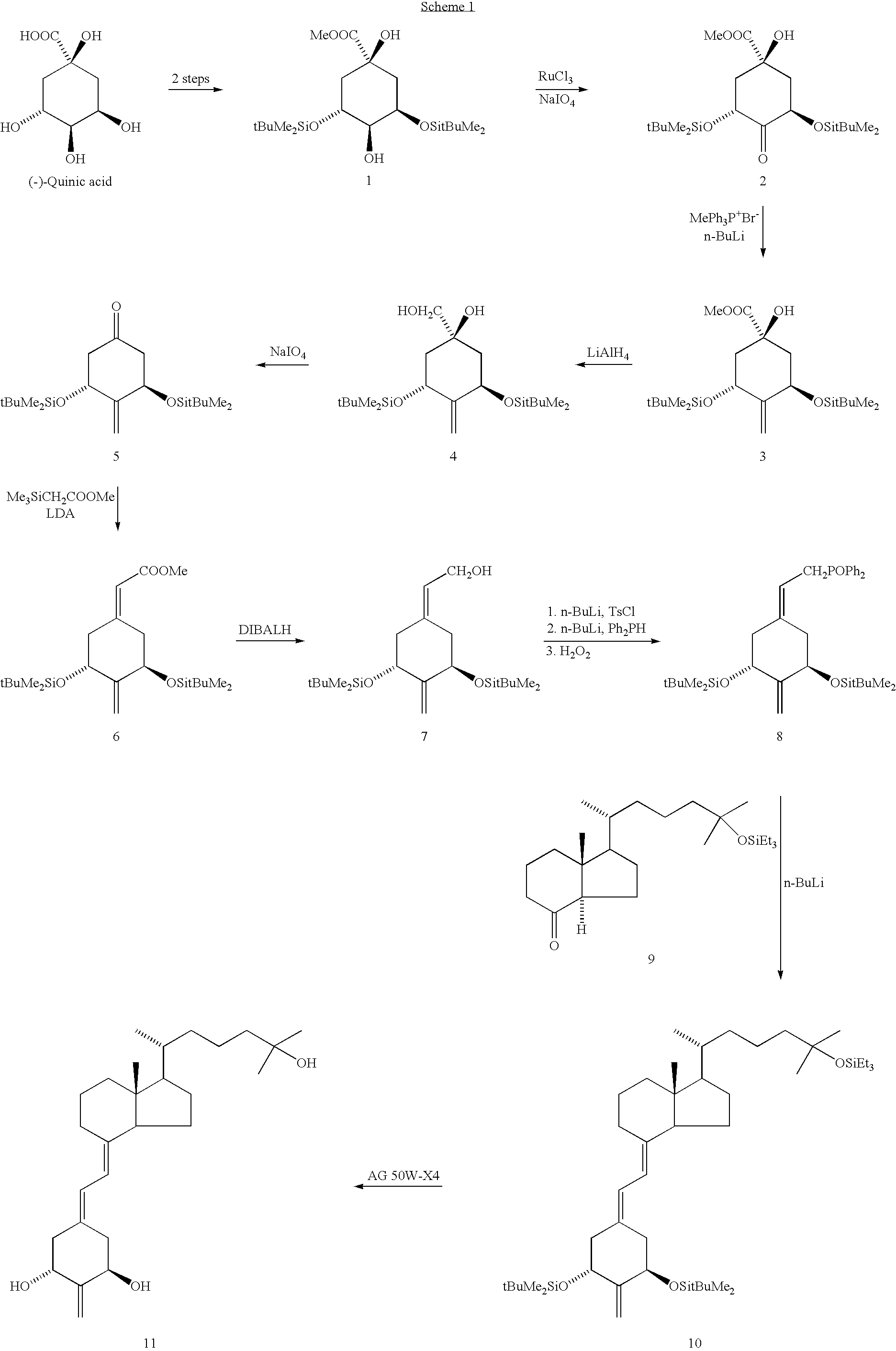

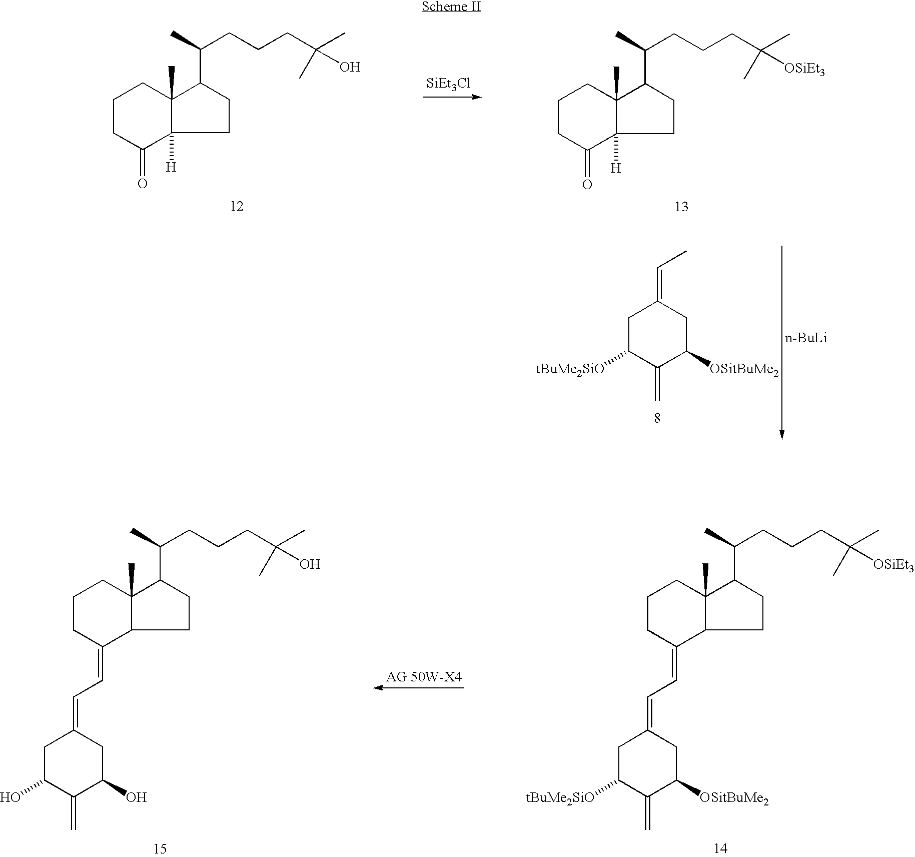

(3-(((4-Tert-Butyl-benzyl)-(pyridine-3-sulfonyl)-amino)-methyl)-phenoxy)-acetic acid is made in a procedure analogous to Example A below with the noted variations, which systhesis is set forth in U.S. Pat. No. 6,498,172.

### (3-(((4-Tert-Butyl-benzyl)-(Pyridine-3-sulfonyl)-amino)-methyl)-phenoxy)-acetic Acid

Step B: N,N-diisopropylethylamine was replaced by triethylamine. ¹H NMR (400 MHz, CDCl₃) δ 8.95 (s, 1H), 8.74 (s, 1H), 7.99 (d, 1H), 7.39 (m, 1H), 7.25 (m, 2H), 7.15 (t, 1H), 7.04 (d, 2H), 6.81 (d, 1H), 6.72 (d, 1H), 6.62 (s, 1H), 4.55 (s, 2H), 4.35 (s, 4H), 1.27 (s, 9H); MS 469 (M+1), 467 (M−1).

### Example A

### 7-((4-Butyl-benzyl)-(Pyridine-3-sulfonyl)-amino)-heptanoic acid

### Step A: Reductive Amination

7-(4-Butyl-benzylamino)-heptanoic acid methyl ester. A solution of 7-amino-heptanoic acid methyl ester hydrochloride, prepared of Preparation 1 (below), (1.12 g, 5.9 mmol), 4-butyl-benzaldehyde (0.915 g, 5.65 mmol) and triethylamine (0.83 mL, 5.98 mmol) in 20 mL MeOH was stirred at room temperature for 3 hours. After cooling to 0° C., NaBH₄ (0.342 g, 9.04 mmol) was added and the reaction was stirred for 15 minutes at room temperature. The mixture was quenched with 1:1 NaHCO₃:H₂O and the MeOH was removed in vacuo. The resulting residue was diluted with CH₂Cl₂ and the organic solution was washed with water and brine, dried over MgSO₄, filtered and concentrated in vacuo to afford the title compound of Step A (1.4 g). ¹H NMR (400 MHz, CDCl₃) δ 7.08-7.38 (m, 4H), 3.62 (s, 2H), 3.29 (s, 3H), 2.52-2.66 (m, 4H), 2.25 (t, 2H), 1.53-1.63 (m, 6H), 1.25-1.40 (m, 6H), 0.85 (t, 3H); MS 306 (M+1).

### Step B: Amide Formation

7-((4-Butyl-benzyl)-(Pyridine-3-sulfonyl)-amino)-heptanoic acid methyl ester. A solution of 7-(4-butyl-benzylamino)-heptanoic acid methyl ester prepared of Example 1, Step A (0.10 g, 0.33 mmol), N,N-diisopropylethylamine (0.85 g 0.66 mmol) and pyridine-3-sulfonyl chloride hydrochloride, prepared of Preparation 2, (0.070 g, 0.33 mmol) in 3 mL CH₂Cl₂ was stirred at room temperature overnight. The mixture was diluted with CH₂Cl₂ and the organic solution was washed with water and brine, dried over MgSO₄, filtered and concentrated in vacuo. The product was purified by flash chromatography on silica gel (10% EtOAc/hexanes to 30% EtOAc/hexanes) to afford the title compound of Step B. ¹H NMR (400 MHz, CDCl₃) δ 9.01 (s, 1H), 8.75 (d, 1H), 8.04 (d, 1H), 7.41 (dd, 1H), 7.23 (m, 4H), 4.30 (s, 2H), 3.62 (s, 3H), 3.08 (t, 2H), 2.55 (t, 2H), 2.19 (t, 2H), 1.10-1.58 (m, 12H), 0.87 (t, 3H); MS 447 (M+1).

### Step C: Ester Hydrolysis

7-((4-Butyl-benzyl)-(pyridine-3-sulfonyl)-amino)-heptanoic acid. A solution of 7-((4-butyl-benzyl)-(pyridine-3-sulfonyl)-amino)-heptanoic acid methyl ester prepared of Example 1, Step B (0.040 g, 0.158 mmol), in 2 mL MeOH and 0.5 mL 2N NaOH was stirred at room temperature overnight. The mixture was quenched with 2N HCl and was diluted with CH₂Cl₂. The organic layer was washed with 1N HCl and water, dried over MgSO₄, filtered and concentrated in vacuo. The product was purified by flash chromatography on silica gel (2% MeOH/CH₂Cl₂ to 5% MeOH/CH₂Cl₂) to afford the title compound (42 mg). ¹H NMR (400 MHz, CDCl₃) δ 9.09 (s, 1H), 8.77 (d, 1H), 8.08 (d, 1H), 7.48 (dd, 1H), 7.09 (m, 4H), 4.32 (s, 2H), 3.12 (s, 2H), 2.55 (t, 2H), 2.25 (t, 2H), 1.12-1.58 (m, 12H), 0.88 (t, 3H); MS 431 (M−1).

### Preparation 1

7-Amino-heptanoic acid methyl ester hydrochloride. A solution of 7-amino-heptanoic acid (3.0 g, 21.0 mmol), in 25 mL MeOH and 2.4 mL concentrated HCl was heated at reflux for 4 hours and was stirred at room temperature for 60 h. The mixture was concentrated in vacuo to afford the title compound (3.3 g). ¹H NMR (400 MHz, CD₃OD) δ 3.62 (s, 3H), 2.89 (m, 2H), 2.31 (t, 2H), 1.62 (m, 4H), 1.37 (m, 4H).

The synthesis of 7-[(4-Butyl-benzyl)-methanesulfonyl-amino]-heptanoic acid and its sodium salt is set forth in U.S. Pat. No. 6,288,120, issued Sep. 11, 200, which synthesis is reproduced below.

### 7-[(4-Butyl-benzyl)-methanesulfonyl-amino]-heptanoic Acid

### Step A: Alkylation

Ethyl 7-[(4-Butyl-benzyl)-methanesulfonyl-amino]-heptanoate. A solution of ethyl-7-methanesulfonyl-amino-heptanoate (250 mg, 1.0 mmol) in DMF (2 mL) was added dropwise to NaH (48 mg, 1.19 mmol, 60% in oil) in DMF at 0° C. After stirring for 45 minutes at room temperature, 1-bromomethyl-4-butyl-benzene (271 mg, 1.19 mmol) was added dropwise. The reaction was stirred for 2 h and the DMF was removed in vacuo. The residue was diluted with CH₂Cl₂ and the organic solution was sequentially washed with 1N HCl (1×), water (2×), and brine (1×). The organic solution was dried over MgSO₄, filtered, and concentrated in vacuo. The product was purified via radial chromatography (15% EtOAc/hexanes to 40% EtOAc/hexanes) to afford the title compound of Step A (379 mg). ¹H NMR (400 MHz, CDCl₃) δ 7.12-7.30 (m, 4H), 4.35 (s, 2H), 4.12 (q, 2H), 3.10-3.19 (m, 2H), 2.80 (s, 3H), 2.60 (t, 2H), 2.25 (t, 2H), 1.46-1.62 (m, 7H), 1.18-1.39 (m, 6H), 0.92 (t, 3H); MS 415 (M+18).

### Step B: Ester Hydrolysis

7-[(4-Butyl-benzyl)-methanesulfonyl-amino]-heptanoic acid. To a solution of the title compound of Step A (379 mg, 0.95 mmol) in MeOH (6 mL) was added NaOH (1.0 mL, 5N). The reaction was stirred at room temperature for 24 h and was acidified with aqueous HCl (1N). The MeOH was removed in vacuo and the residue was dissolved in CH₂Cl₂. The organic solution was washed sequentially with HCl (1N, 1×), water (2×), and brine (1×). The organic solution was dried with MgSO₄, filtered, and concentrated in vacuo. Purification by radial chromatography (CH₂Cl₂ to 6% MeOH/CH₂Cl₂) provided the title compound (356 mg). ¹H NMR (400 MHz, CDCl₃) δ 7.30-7.12 (m, 4H), 4.35 (s, 2H), 3.10-3.19 (m, 2H), 2.80 (s, 3H), 2.60 (t, 2H), 2.31 (t, 2H), 1.48-1.65 (m, 7H), 1.20-1.40 (m, 6H). 0.97 (t, 3H); MS 387 (M+18).

7-[(4-Butyl-benzyl)-methanesulfonyl-amino]-heptanoic acid sodium salt Following the procedure described above, the sodium salt was generated. The sodium salt was stirred in 10% EtOH in EtOAc at 65° C. for 20 h. The mixture was cooled to room temperature and was filtered to provide a white solid. mp 137° C.; ¹H NMR (400 MHz, CD₃OD) δ 7.27 (d, 2H), 7.15 (d, 2H), 4.32 (s, 2H), 3.12 (t, 2H), 2.85 (s, 3H), 2.60 (t, 2H), 2.09 (t, 2H), 1.60-1.20 (m, 12H), 0.92 (t, 3H).

The Synthesis of {3-[(4-Tert-Butyl-benzyl)-(pyridine-3-sulfonyl)-amino]-methyl}-phenoxy acetic acid sodium salt is set forth in U.S. Pat. No. 6,498,172 and recited below.

### {3-[(4-Tert-Butyl-benzyl)-(pyridine-3-sulfonyl)-amino]-methyl}-phenoxy Acetic Acid Sodium Salt

### Step A: Reductive Amination

{3-[(4-Tert-Butyl-benzylamino)-methyl]-phenoxy}acetic acid tert-butyl ester. To a solution of (3-aminomethyl-phenoxy)-acetic acid tert-butyl ester prepared in Step C of Preparation 2, (0.497 g, 2.09 mmol) in MeOH (8 mL) was added 4-tert-butylbenzaldehyde (0.33 mL, 1.97 mmol), and the mixture was stirred at room temperature for 2 h. The solution was cooled to 0° C. and sodium borohydride (0.119 g, 3.15 mmol) was added in one portion. The mixture was stirred for 10 min, and a 1:1 solution of water:aqueous saturated sodium bicarbonate was added to the solution. The product was extracted into CH₂Cl₂ (3×) and the combined organic solutions were dried (MgSO₄) and concentrated in vacuo. The product was purified via silica gel chromatography (EtOAc followed by 5% MeOH in CH₂Cl₂) to give the title compound of Step A (0.691 g) as a clear oil. ¹H NMR (400 MHz, CDCl₃) δ 7.30-7.38 (m, 2H), 7.19-7.28 (m, 3H), 6.87-6.96 (m, 2H), 6.77 (d, 1H), 4.50 (s, 2H), 3.77 (s, 2H), 3.75 (s, 2H), 1.46 (s, 9H), 1.30 (s, 9H); MS 384 (M+1).

### Step B: Amide Formation

{3-[(4-Tert-Butyl-benzyl)-(pyridine-3-sulfonyl)-amino]-methyl}-phenoxy acetic acid tert-butyl ester. To a solution of {3-[(4-tert-butyl-benzylamino)-methyl]-phenoxy}acetic acid tert-butyl ester (10.0 g, 26.1 mmol), prepared in Step A, in CH₂Cl₂ (75 mL) at 0° C. was added triethylamine (8.0 mL, 57.4 mmol), and pyridine-3-sulfonyl chloride hydrochloride (6.10 g, 28.7 mmol), of Preparation 2. The mixture was stirred for 0.5 h, the ice bath was removed, and the mixture was stirred for an additional 1.5 h. A 1:1 solution of water:aqueous saturated sodium bicarbonate was added to the solution, and the product was extracted into CH₂Cl₂ (3×). The combined organic solutions were dried over MgSO₄ and concentrated in vacuo and the product was purified via silica gel chromatography (2:1 Hex:EtOAc) to give the title compound of Step B (11.0 g) as a clear oil. ¹H NMR (400 MHz, CDCl₃) δ 9.01 (s, 1H), 8.75 (d, 1H), 7.97 (d, 1H), 7.38 (m, 1H), 7.11-7.23 (m, 3H), 6.97 (d, 2H), 6.71 (d, 1H), 6.65 (d, 1H), 6.60 (s, 1H), 4.40 (s, 2H), 4.32 (s, 4H), 1.48 (s, 9H), 1.26 (s, 9H); MS 525 (M+1).

### Step C: Ester Hydrolysis

{3-[(4-Tert-Butyl-benzyl)-(pyridine-3-sulfonyl)-amino]-methyl}-phenoxy acetic acid. To a solution of {3-[(4-tert-butyl-benzyl)-(pyridine-3-sulfonyl)-amino]-methyl}-phenoxy acetic acid tert-butyl ester (11.0 g, 21.0 mmol), prepared in Step B, in CH₂Cl₂ (50 mL) at 0° C. was added trifluoroacetic acid (25 mL). After 10 min the ice bath was removed and the mixture was stirred for an additional 1.5 h. An additional 5 mL of trifluoroacetic acid was added, the mixture was stirred for 30 min, and the reaction was concentrated in vacuo. The residue was azeotroped with CH₂Cl₂ (3×), and the resulting oil was partitioned between water and EtOAc. The aqueous phase was adjusted to pH 5.0 with 1N NaOH and the resulting precipitated solid (4.86 g) was collected by filtration. The filtrate layers were separated and the aqueous layer was extracted with EtOAc (2×). The combined organic solutions were dried over MgSO₄ and concentrated in vacuo to give a white foam (2.64 g). The precipitated solid and the white foam were combined and recrystallized from ethanol to give the title compound (5.68 g) as a white solid. ¹H NMR (400 MHz, CD₃OD) δ 8.91 (s, 1H), 8.71 (d, 1H), 8.15 (d, 1H), 7.54 (m, 1H), 7.22 (d, 2H), 7.11 (t, 1H), 7.04 (d, 2H), 6.71-6.92 (m, 2H), 6.65 (s, 1H), 4.50 (s, 2H), 4.36 (s, 4H), 1.25 (s, 9H); MS 469 (M+1).

### Step D: Salt Formation

{3-[(4-tert-Butyl-benzyl)-(pyridine-3-sulfonyl)-amino]-methyl}-phenoxy acetic acid sodium salt. To a solution of {3-[(4-tert-butyl-benzyl)-(pyridine-3-sulfonyl)-amino]-methyl}-phenoxy acetic acid (5.68 g, 12.13 mmol), prepared in Step C, in 10:1 MeOH:water (66 mL) was added sodium bicarbonate (1.02 g, 12.13 mmol) and the mixture was stirred for 18 h at room temperature. The mixture was azeotroped with ethanol and concentrated in vacuo to give the title compound (5.95 g) as a white solid. ¹H NMR (400 MHz, CD₃OD) δ 8.88 (s, 1H), 8.71 (d, 1H), 8.13 (d, 1H), 7.52 (m, 1H), 7.24 (d, 2H), 7.04-7.11 (m, 3H), 6.78 (d, 1H), 6.68 (m, 2H), 4.37 (s, 2H), 4.35 (s, 2H), 4.25 (s, 2H), 1.25 (s, 9H); MS 469 (M+1).

### Preparation 2

### (3-Aminomethyl-phenoxy)-acetic Acid Tert-Butyl Ester

### Step A

(3-Formyl-phenoxy)-acetic acid tert-butyl ester. To a solution of 3-hydroxybenzaldehyde (5.00 g, 40.9 mmol) in DMF (40 mL) was added 1M potassium tert-butoxide in tert-butanol (40.9 mL, 40.9 mmol). The reaction was stirred for 2 minutes and tert-butyl bromoacetate (6.61 mL, 40.9 mmol) was added. The reaction was stirred for 1 hour and was quenched with 200 mL water. The product was extracted into EtOAc and the organic solution was washed with water, dried over MgSO₄, filtered, and concentrated in vacuo. Purification via flash chromatography on silica gel (9:1 hexanes:EtOAc) afforded the title compound of Step A as a clear oil (3.53 g). ¹H NMR (400 MHz, CDCl₃) δ 9.94 (s, 1H), 7.48 (m, 2H), 7.32 (s, 1H), 7.21 (m, 1H), 4.56 (s, 2H), 1.45 (s, 9H).

### Step B

(3-(Hydroxyimino-methyl)-phenoxy)-acetic acid tert-butyl ester. To a solution of (3-formyl-phenoxy)-acetic acid tert-butyl ester prepared of Preparation 20, Step A (2.05 g, 8.68 mmol) in MeOH (30 mL) was added NH₂OH.HCl (0.66 g, 9.54 mmol) and pyridine (3.5 mL, 43.4 mmol) and the reaction was stirred for 2 hours. The MeOH was removed in vacuo and the residue was diluted with EtOAc and 1N HCl. The layers were separated and the aqueous solution was washed with EtOAc. The combined organic layers were dried over MgSO₄, filtered and concentrated in vacuo to afford the title compound of Step B (1.99 g). ¹H NMR (400 MHz, CDCl₃) δ 8.07 (s, 1H), 7.23-7.28 (m, 2H), 7.12 (m, 1H), 6.93 (d, 1H), 4.51 (s, 2H), 1.46 (s, 9H).

### Step C

(3-Aminomethyl-phenoxy)-acetic acid tert-butyl ester. To a solution of (3-(hydroxyimino-methyl)-phenoxy)-acetic acid tert-butyl ester prepared of Preparation 20, Step B (2.25 g, 5.96 mmol) in EtOH (10 mL) was added Raney Nickel (about 1 g, washed with water followed by EtOH) in 100 mL EtOH. Additional EtOH (90 mL) was required for the transfer. Ammonium hydroxide (10 mL) was added and the mixture was shaken under 45 psi of H₂ for 4 hours. The catalyst was removed via filtration through Celite® and the solution was concentrated to a clear oil. Purification via flash chromatography on silica gel (96.5/3.5/0.1 to 9/1/0.1 CH₂Cl₂/MeOH/NH₄OH) afforded the title compound as a yellow oil. ¹H NMR (400 MHz, CDCl₃) δ 7.23 (m, 1H), 6.92 (m, 2H), 6.72 (d, 1H), 4.50 (s, 2H), 3.82 (s, 2H), 1.96 (m, 2H), 1.46 (s, 9H); MS 238 (M+1).

The Synthesis of 5-(3-{2S-[3R-Hydroxy-4-(3-trifluoromethyl-phenyl)-butyl]-5-oxo-pyrrolidin-1-yl}-propyl)-thiophene-2-carboxylic acid is set forth in U.S. Pat. No. 6,552,067, which synthesis is reproduced below:

### 5-(3-{2S-[3R-Hydroxy-4-(3-trifluoromethyl-phenyl)butyl]-5-oxo-pyrrolidin-1-yl}-propyl)-thiophene-2-carboxylic Acid

Step A: 5-(3-{2-Oxo-5R-[3-oxo-4-(3-trifluoromethyl-phenyl)-but-1-enyl]-pyrrolidin-1-yl}-propyl)-thiophene-2-carboxylic acid methyl ester. Analogous to the procedure described for Example 2A, Step B, the anion derived from [2-oxo-3-(3-trifluoromethyl-phenyl)-propyl]-phosphonic acid dimethyl ester (5.026 g, 17.0 mmol) and NaH (60% by weight in oil, 750 mg, 18.8 mmol) was reacted with 5-[3-(2R-formyl-5-oxo-pyrrolidin-1-yl)-propyl]-thiophene-2-carboxylic acid methyl ester (assumed 18.8 mmol) over 24 h. Purification by medium pressure chromatography (15% acetone in toluene to 20% acetone in toluene) provided 5-(3-{2-oxo-5R-[3-oxo-4-(3-trifluoromethyl-phenyl)-but-1-enyl]-pyrrolidin-1-yl}-propyl)-thiophene-2-carboxylic acid methyl ester (4.02 g). ¹H NMR (CDCl₃) δ7.61 (d, 1H), 7.54 (d, 1H), 7.45 (m, 2H), 7.37 (d, 1H), 6.79 (d, 1H), 6.66 (dd, 1H), 6.20 (d, 1H), 4.16 (m, 1H), 3.90 (s, 2H), 3.84 (s, 3H), 3.60 (m, 1H), 2.89-2.78 (m, 3H), 2.48-2.31 (m, 2H), 2.23 (m, 1H), 1.82 (m, 3H).

Step B: 5-(3-[2R-[3S-Hydroxy-4-(3-trifluoromethyl-phenyl)-but-1-enyl]-5-oxo-pyrrolidin-1-yl]-propyl)-thiophene-2-carboxylic acid methyl ester. Analogous to the procedure described for Example 2A, Step C, 5-(3-{2-oxo-5R-[3-oxo-4-(3-trifluoromethyl-phenyl)-but-1-enyl]-pyrrolidin-1-yl}-propyl)-thiophene-2-carboxylic acid methyl ester (2.63 g, 5.91 mmol) was reduced with catecholborane (1M in THF, 18.8 mL, 18.8 mmol) in the presence of (R)-2-methyl-CBS-oxazaborolidine (1M in toluene, 0.94 mL, 0.94 mmol) at −45° C. over 18 h. The reaction was quenched by addition of 1N HCl and the mixture was stirred for 40 minutes. The organic solution was washed consecutively with ice cold 1N NaOH (3 times), 1N HCl (1 time), water (1 time), and brine. The organic solution was dried (MgSO₄), filtered, and concentrated. Purification by medium pressure chromatography (10% acetone in toluene to 20% acetone in toluene) provided 5-(3-{2R-[3S-hydroxy-4-(3-trifluoromethyl-phenyl)-but-1-enyl]-5-oxo-pyrrolidin-1-yl}-propyl)-thiophene-2-carboxylic acid methyl ester (3 g) as an approximate 4:1 ratio of 3S:3R alcohol diastereomers by ¹H NMR. ¹H NMR (CDCl₃) δ7.60 (d, 1H), 7.50 (d, 1H), 7.41 (m, 3H), 6.79 (d, 1H), 5.70 (dd, 1H), 5.48 (dd, 1H), 4.41 (m, 1H), 4.00 (m, 1H), 3.81 (s, 3H), 3.50 (m, 1H), 2.86-2.77 (m, 5H), 2.42-2.26 (m, 2H), 2.16 (m, 1H), 1.81 (m, 2H), 1.72-1.54 (m, 2H).

Step C: 5-(3-{2S-[3R-Hydroxy-4-(3-trifluoromethyl-phenyl)-butyl]-5-oxo-pyrrolidin-1-yl}-propyl)-thiophene-2-carboxylic acid methyl ester. Analogous to the procedure described for Example 2A, Step D, a mixture of 5-(3-{2R-[3S-hydroxy-4-(3-trifluoromethyl-phenyl)-but-1-enyl]-5-oxo-pyrrolidin-1-yl}-propyl)-thiophene-2-carboxylic acid methyl ester (3 g) and 10% palladium on carbon (400 mg) in MeOH (70 mL) was hydrogenated on a Parr shaker at 50 psi for 16 h. Purification by medium pressure chromatography (20% EtOAc in hexanes to 70% EtOAc in hexanes) provided 5-(3-{2S-[3R-hydroxy-4-(3-trifluoromethyl-phenyl)-butyl]-5-oxo-pyrrolidin-1-yl}-propyl)-thiophene-2-carboxylic acid methyl ester (2.26 g). ¹H NMR (CDCl₃) δ7.61 (d, 1H), 7.52-7.38 (m, 4H), 6.81 (d, 1H), 3.83 (m, 4H), 3.63 (m, 2H), 3.00 (m, 1H), 2.85 (m, 3H), 2.74 (m, 1H), 2.34 (m, 2H), 2.10 (m, 1H), 1.98-1.45 (m, 08H).

Step D: 5-(3-{2S-[3R-Hydroxy-4-(3-trifluoromethyl-phenyl)-butyl]-5-oxo-pyrrolidin-1-yl}-propyl)-thiophene-2-carboxylic acid. Analogous to the procedure described for Example 2A, Step E, 5-(3-{2S-[3R-hydroxy-4-(3-trifluoromethyl-phenyl)-butyl]-5-oxo-pyrrolidin-1-yl}-propyl)-thiophene-2-carboxylic acid methyl ester (625 mg) was hydrolyzed with 2N NaOH in MeOH (20 mL) at room temperature over 24 h to provide the title compound of Example 3M (599 mg). ¹H NMR (CDCl₃) δ7.67 (d, 1H), 7.51-7.38 (m, 4H), 6.84 (d, 1H), 3.85 (m, 1H), 3.63 (m, 2H), 3.02 (m, 1H), 2.85 (m, 3H), 2.75 (m, 1H), 2.37 (m, 2H), 2.11 (m, 1H), 2.00-1.45 (m, 8H); MS 470.2 (M+1), 468.2 (M−1).

### Example 2A

### 7-{2S-[3R-Hydroxy-4-(3-methoxymethyl-phenyl)-butyl]-5-oxo-pyrrolidin-1-yl}-heptanoic Acid

Step A: 7-(2R-Formyl-5-oxo-pyrrolidin-1-yl)-heptanoic acid ethyl ester. To a solution of 7-(2R-hydroxymethyl-5-oxo-pyrrolidin-1-yl)-heptanoic acid ethyl ester (1.63 g, 6.01 mmol) in anhydrous benzene (50 mL) was added 1-(3-dimethylaminopropyl)-3-ethylcarbodiimide hydrochloride (3.46 g, 18.03 mmol) and DMSO (1.5 mL, 24.04 mmol). The solution was cooled to 0° C. and pyridinium trifluoroacetate (1.28 g, 6.61 mmol) was added. The reaction mixture was stirred at 0° C. for 15 minutes and at room temperature for 2 h. The solution was decanted from the oily residue. The residue was washed with benzene (3×) and the combined benzene washes were concentrated in vacuo to provide 7-(2R-formyl-5-oxo-pyrrolidin-1-yl)-heptanoic acid ethyl ester, which was used in Step B without further purification.

Step B: 7-{2R-[4-(3-Methoxymethyl-phenyl)-3-oxo-but-1-enyl]-5-oxo-pyrrolidin-1-yl}-heptanoic acid ethyl ester. To a solution of [3-(3-methoxymethyl-phenyl-2-oxo-propyl]-phosphonic acid diethyl ester (1.715 g, 5.46 mmol) in THF (43 mL) at 0° C. was added NaH (60% by weight in oil, 240 mg, 6.00 mmol) portionwise. The reaction mixture was stirred at room temperature for 45 minutes. The reaction mixture was cooled to 0° C. and a solution of 7-(2R-formyl-5-oxo-pyrrolidin-1-yl)-heptanoic acid ethyl ester (prepared in Step A, assumed 6.01 mmol) in THF (32 mL) was added dropwise. The reaction mixture was stirred at 0° C. for 15 minutes and at room temperature for 24 h. The reaction mixture was cooled to 0° C. and acetic acid was added until a pH of 5 was achieved. EtOAc and water were added and the aqueous solution was washed with EtOAc (3×). The organic solutions were combined, washed with water, dried (MgSO₄), filtered and concentrated. The residue was purified by medium pressure chromatography eluting with a solvent gradient (2:1 hexanes:EtOAc to 1:1 hexanes:EtOAc to 1% MeOH in CH₂Cl₂ to 3% MeOH in CH₂Cl₂) to provide 7-{2R-[4-(3-methoxymethyl-phenyl)-3-oxo-but-1-enyl]-5-oxo-pyrrolidin-1-yl}-heptanoic acid ethyl ester (1.4 g). ¹H NMR (CDCl₃) δ7.29 (m, 1H), 7.22 (m, 1H), 7.16 (s, 1H), 7.09 (d, 1H), 6.62 (dd, 1H), 6.19 (d, 1H), 4.41 (s, 2H), 4.10 (m, 3H), 3.82 (s, 2H), 3.51 (m, 1H), 3.36 (s, 3H), 2.67 (m, 1H), 2.43-2.18 (m, 5H), 1.75 (m, 1H), 1.56 (m, 2H), 1.42-1.17 (m, 9H).

Step C: 7-{2R-[3S-Hydroxy-4-(3-methoxymethyl-phenyl)-but-1-enyl]-5-oxo-pyrrolidin-1-yl}-heptanoic acid ethyl ester. To a solution of 7-{2R-[4-(3-methoxymethyl-phenyl)-3-oxo-but-1-enyl]-5-oxo-pyrrolidin-1-yl}-heptanoic acid ethyl ester (1.40 g, 3.26 mmol) in anhydrous CH₂Cl₂ (200 mL) was added (R)-2-methyl-CBS-oxazaborolidine (1M in toluene, 0.49 mL, 0.49 mmol) and the solution was cooled to −45° C. The reaction mixture was stirred for 20 minutes and catecholborane (1M in THF, 9.8 mL, 9.8 mmol) was added. The reaction mixture was stirred for 24 h at −45° C. and THF (100 mL) and HCl (1N, 100 mL) were added. The reaction mixture was stirred at room temperature for 24 h and at 40-45° C. for 1.5 h. The solution was diluted with CH₂Cl₂ and water and the layers were separated. The organic solution was cooled to 0° C. and was washed with ice-cold NaOH (0.5N) followed by brine. The organic solution was again washed with ice-cold NaOH (0.5 N) followed by brine and was dried (MgSO₄), filtered and concentrated. Purification by medium pressure chromatography eluting with a solvent gradient (5:1 hexanes:EtOAc to 2:1 hexanes:EtOAc to 1:1 hexanes:EtOAc to EtOAc to 2% MeOH in CH₂Cl₂) provided 7-{2R-[3S-hydroxy-4-(3-methoxymethyl-phenyl)-but-1-enyl]-5-oxo-pyrrolidin-1-yl}-heptanoic acid ethyl ester (1.2 g) as an approximate 12:1 mixture of 3S:3R alcohol diasteromers by HPLC analysis. ¹H NMR (CDCl₃) (selected peaks) 67.26-7.07 (m, 4H), 5.67 (m, 1H), 5.43 (m, 1H), 4.39 (s, 2H), 4.36 (m, 1H), 4.06 (q, 2H), 3.98 (m, 1H), 3.41 (m, 1H), 3.35 (s, 3H); MS 432.3 (M+1), 430.3 (M−1).

Step D: 7-{2S-[3R-Hydroxy-4-(3-methoxymethyl-phenyl)-butyl]-5-oxopyrrolidin-1-yl}-heptanoic acid ethyl ester. To a solution of 7-{2R-[3S-hydroxy-4-(3-methoxymethyl-phenyl)-but-1-enyl]-5-oxo-pyrrolidin-1-yl}-heptanoic acid ethyl ester (1.2 g, 2.78 mmol) in EtOH (100 mL) was added 10% palladium on carbon (120 mg). The reaction mixture was hydrogenated on a Parr shaker at 45 psi for 24 h. The catalyst was removed via filtration through Celite® with the aid of EtOH. Purification by medium pressure chromatography eluting with a solvent gradient (CH₂Cl₂ to 2% MeOH in CH₂Cl₂ to 5% MeOH in CH₂Cl₂) (2×) provided 7-{2S-[3R-hydroxy-4-(3-methoxymethyl-phenyl)-butyl]-5-oxo-pyrrolidin-1-yl}-heptanoic acid ethyl ester (1.1 g). ¹H NMR (CDCl₃) δ7.28 (m, 1H), 7.18 (m, 2H), 7.11 (m, 1H), 4.42 (s, 2H), 4.08 (q, 2H), 3.82 (m, 1H), 3.58 (m, 2H), 3.38 (s, 3H), 2.84 (m, 2H), 2.66 (m, 1H), 2.41-2.23 (m, 4H), 2.08 (m, 1H), 1.78 (m, 1H), 1.64-1.37 (m, 9H), 1.28 (m, 4H), 1.22 (t, 3H).

Step E: 7-{2S-[3R-Hydroxy-4-(3-methoxymethyl-phenyl)-butyl]-5-oxo-pyrrolidin-1-yl}-heptanoic acid. To a solution of 7-{2S-[3R-hydroxy-4-(3-methoxymethyl-phenyl)-butyl]-5-oxo-pyrrolidin-1-yl}-heptanoic acid ethyl ester (1.1 g, 2.53 mmol) in EtOH (32 mL) was added NaOH (6N, 16 mL). The reaction mixture was stirred for 24 h and 1N HCl was added to obtain a pH of about 2. Brine and CH₂Cl₂ were added and the layers were separated. The aqueous solution was washed with 5% MeOH in CH₂Cl₂ (2 times). The combined organic layers were dried (MgSO₄), filtered and concentrated to provide the title compound of Example 2A (990 mg). ¹H NMR (CDCl₃) δ7.28 (m, 1H), 7.18 (m, 2H), 7.11 (m, 1H), 4.43 (s, 2H), 3.83 (m, 1H), 3.57 (m, 2H), 3.40 (s, 3H), 2.91 (m, 1H), 2.79 (m, 1H), 2.66 (m, 1H), 2.43-2.25 (m, 4H), 2.10 (m, 1H), 1.83 (m, 1H), 1.66-1.22 (m, 13H); MS 406.3 (M+1), 404.3 (M−1).

1. A pharmaceutical composition comprising the compound 2-methylene-19-nor-20(S)-1α,25-dihydroxyvitamin D₃ and an EP₂ or EP₄ agonist or a pharmaceutically acceptable salt or prodrug thereof.

2. A composition of claim 1 wherein the EP₂ or EP₄ agonist is an EP₂ agonist and the EP₂agonist is (3-(((4-tert-butyl-benzyl)-(pyridine-3-sulfonyl)-amino)-methyl)-phenoxy)-acetic acid or a pharmaceutically acceptable salt thereof.

3. A composition of claim 2 wherein the (3-(((4-tert-butyl-benzyl)-(pyridine-3-sulfonyl)-amino)-methyl)-phenoxy)-acetic acid is in the form of a sodium salt.

4. A method of treating senile osteoporosis, postmenopausal osteoporosis, bone fracture, bone graft, breast cancer, prostate cancer, obesity, osteopenia, male osteoporosis, frailty, muscle damage or sarcopenia, the method comprising administering to a patient in need thereof a therapeutically effective amount of 2-methylene-19-nor-20(S)-1α,25-dihydroxyvitamin D₃ and an EP₂ or EP₄ agonist or a pharmaceutically acceptable salt or prodrug thereof.

5. The method of claim 4 wherein the 2-methylene-19-nor-20(S)-1α,25-dihydroxyvitamin D₃ and the EP₂ or EP₄ agonist or a pharmaceutically acceptable salt or prodrug thereof are administered orally.

6. The method of claim 4 wherein the 2-methylene-19-nor-20(S)-1α,25-dihydroxyvitamin D₃ and the EP₂ or EP₄ agonist or a pharmaceutically acceptable salt or prodrug thereof agonist are administered parenterally.

7. The method of claim 4 wherein the 2-methylene-19-nor-20(S)-1α,25-dihydroxyvitamin D₃ and the EP₂ or EP₄ agonist or a pharmaceutically acceptable salt or prodrug thereof are administered transdermally.

8. The method of claim 4 wherein the 2-methylene-19-nor-20(S)-1α,25-dihydroxyvitamin D₃ and the EP₂ or EP₄ agonist or a pharmaceutically acceptable salt or prodrug thereof are administered substantially simultaneously.

9. The method of claim 4 wherein a bone fracture is treated.

10. The method of claim 4 wherein postmenopausal osteoporosis is treated.

11. A method of treating a bone fracture, the method comprising administering to a patient in need thereof a therapeutically effective amount of 2-methylene-19-nor-20(S)-1α,25-dihydroxyvitamin D₃ and (3-(((4-tert-butyl-benzyl)-(pyridine-3-sulfonyl)-amino)-methyl)-phenoxy)-acetic acid or a pharmaceutically acceptable salt or prodrug thereof.

12. The method of claim 11 wherein the (3-(((4-tert-butyl-benzyl)-(pyridine-3-sulfonyl)-amino)-methyl)-phenoxy)-acetic acid is in the form of a sodium salt.
